(19) United States
(12) Patent Application Publication
(10) Pub. No.: US 2021/0128537 A1
(43) Pub. Date: May 6, 2021

(54) T-TYPE CALCIUM CHANNEL MODULATORS AND METHODS OF USE THEREOF

(71) Applicants: Praxis Precision Medicines, inc., Cambridge, MA (US); Monash University, Clayton, VIC (AU)

(72) Inventors: Kiran Reddy, Boston, MA (US); Terence John O'Brien, Clayton, VIC (AU); Pablo Miguel Casillas-Espinosa, Clayton, VIC (AU); Kim Powell, Clayton, VIC (AU)

(21) Appl. No.: 16/472,576

(22) PCT Filed: Dec. 21, 2017
(86) PCT No.: PCT/US2017/000097

§ 371 Date: Jun. 21, 2019

Related U.S. Application Data
(60) Provisional application No. 62/437,589, filed on Dec. 21, 2016.

(51) Int. Cl. A61K 31/445; A61K 9/00; A61K 31/19; A61K 31/4015; A61K 31/53; A61P 25/08; A61P 25/24

(52) U.S. Cl. CPC A61K 31/445; A61K 9/0053; A61K 31/19; A61P 25/24; A61K 31/53; A61P 25/08; A61K 31/4015

(57) ABSTRACT

Described herein are compounds useful for preventing and/or treating a disease or condition relating to aberrant function of a T-type calcium channel, such as epilepsy and epilepsy syndromes (e.g., absence seizures, juvenile myoclonic epilepsy, or a genetic epilepsy) and mood disorders. The present invention further comprises methods for modulating the function of a T-type calcium channel.

### CROSS REFERENCE TO RELATED APPLICATIONS

The present application claims priority to U.S. Provisional Application No. 62/437,589, filed on Dec. 21, 2016, the entire disclosure of which is incorporated herein by reference.

### BACKGROUND

T-type calcium channels are low-voltage activated ion channels that mediate the influx of calcium into cells. Aberrant function of these ion channels is associated with several diseases or conditions, including pain, epilepsy, or an epilepsy syndrome (e.g., absence seizures and juvenile myoclonic epilepsy). Accordingly, compounds that selectively modulate T-type calcium channel in mammals may be useful in treatment of such disease states.

### SUMMARY OF THE INVENTION

Described herein are compounds useful for preventing and/or treating a disease or condition relating to aberrant function of a T-type calcium channel, such as epilepsy and epilepsy syndromes (e.g., absence seizures, juvenile myoclonic epilepsy, or a genetic epilepsy) and mood disorders. The present invention further comprises methods for modulating the function of a T-type calcium channel.

In one aspect, the present invention features a method of treating absence seizures in a subject in need thereof, wherein the method comprises administering, e.g., orally administering, to the subject a T-type calcium channel modulator, e.g., a T-type calcium channel activator, a compound described herein, or a pharmaceutically acceptable salt thereof. In some embodiments, the dosage of the compound is about 10 mg or more; or the compound is administered concurrently with an anti-epileptic drug (e.g, ethosuximide, valproic acid, or lamotrigine); or the absence seizures are refractory absence seizures. In some embodiments, the absence seizures are refractory absence seizures. In some embodiments, the absence seizures are refractory to an anti-epileptic drug (e.g., ethosuximide, valproic acid, or lamotrigine).

In some embodiments, the subject has epilepsy. In some embodiments, the absence seizures are atypical absence seizures. In some embodiments, the absence seizures comprise adult absence seizures, juvenile absence seizures, or childhood absence seizures.

In another aspect, the present invention features a method of treating juvenile myoclonic epilepsy in a subject in need thereof, wherein the method comprises administering, e.g., orally administering, to the subject a T-type calcium channel modulator, e.g., a T-type calcium channel activator, a compound described herein, or a pharmaceutically acceptable salt thereof. In some embodiments, the dosage of the compound is about 10 mg or more; or the compound is administered concurrently with an anti-epileptic drug (e.g, ethosuximide, valproic acid, or lamotrigine); or the juvenile myoclonic epilepsy is refractory juvenile myoclonic epilepsy.

In another aspect, the present invention features a method of treating a genetic epilepsy in a subject in need thereof, wherein the method comprises administering, e.g., orally administering, to the subject a T-type calcium channel modulator, e.g., a T-type calcium channel activator, a compound described herein, or a pharmaceutically acceptable salt thereof. In some embodiments, the dosage of the compound is about 10 mg or more; or the compound is administered concurrently with an anti-epileptic drug (e.g, ethosuximide, valproic acid, or lamotrigine); or the genetic epilepsy is a refractory genetic epilepsy.

In another aspect, the present invention features a method of treating status epilepticus in a subject in need thereof, wherein the method comprises administering, e.g., orally or parenterally (e.g., injection, intravenous or intramuscular) administering, to the subject a T-type calcium channel modulator, e.g., a T-type calcium channel activator, a compound described herein, or a pharmaceutically acceptable salt thereof. In some embodiments, the dosage of the compound is about 10 mg or more; or the compound is administered concurrently with an anti-epileptic drug (e.g, ethosuximide, valproic acid, or lamotrigine).

In another aspect, the present invention features a method of treating a mood disorder in a subject in need thereof, wherein the method comprises administering, e.g., orally administering, to the subject a T-type calcium channel modulator, e.g., a T-type calcium channel activator, a compound described herein, or a pharmaceutically acceptable salt thereof. In some embodiments, the dosage of the compound is about 10 mg or more; or the compound is administered concurrently with an anti-epileptic drug (e.g, ethosuximide, valproic acid, or lamotrigine).

In another aspect, the present invention features a method of modulating a T-type calcium channel in a subject, wherein the method comprises administering, e.g., orally administering, to the subject a T-type calcium channel modulator, e.g., a T-type calcium channel activator, a compound described herein, or a pharmaceutically acceptable salt thereof. In some embodiments, the dosage of the compound is about 10 mg or more; or the compound is administered concurrently with an anti-epileptic drug (e.g, ethosuximide, valproic acid, or lamotrigine).

In another aspect, the present invention features a method of enhancing the potency of an inactivated T-type calcium channel in a subject (e.g., relative to a reference standard), wherein the method comprises administering, e.g., orally administering, to the subject a T-type calcium channel modulator, e.g., a T-type calcium channel activator, a compound described herein, or a pharmaceutically acceptable salt thereof. In some embodiments, the dosage of the compound is about 10 mg or more; or the compound is administered concurrently with an anti-epileptic drug (e.g, ethosuximide, valproic acid, or lamotrigine).

In any and all aspects, in some embodiments, the compound is Z944:

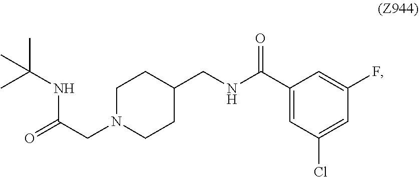 [SMILES: CC(C)(C)NC(=O)CN1CCC(CNC(=O)c2cc(F)cc(Cl)c2)CC1]

or a pharmaceutically acceptable salt thereof.

In any and all aspects, in some embodiments, the compound is Z941:

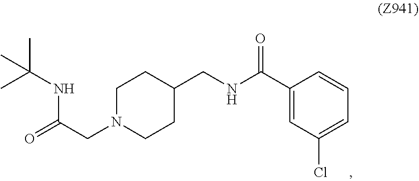 [SMILES: CC(C)(C)NC(=O)CN1CCC(CNC(=O)c2cccc(Cl)c2)CC1]

or a pharmaceutically acceptable salt thereof.

In some embodiments, the subject experiences at least one seizure per day. In some embodiments, the subject experiences at least one seizure per day comprising at least 1-6 Hz (e.g., 3-4 Hz) SWD. In some embodiments, the subject experiences at least one seizure per day lasting about 5 seconds or more (e.g., about 10 seconds or more, about 15 seconds or more, about 20 seconds or more, about 30 seconds or more, or about 1 minute or more). In some embodiments, the subject experiences a decrease in seizure SWDs upon administration of the compound.

In some embodiments, the compound is administered concurrently with an anti-epileptic drug (e.g, ethosuximide, valproic acid, or lamotrigine). In some embodiments, the concurrent administration comprises simultaneous administration, or administration of the compound before or after an anti-epileptic drug (e.g., ethosuximide, valproic acid, or lamotrigine).

In some embodiments, the compound is administered daily. In some embodiments, the compound is administered twice daily. In some embodiments, the compound is administered daily for at least one week.

In some embodiments, the dosage of the compound of is greater than 10 mg. In some embodiments, the dosage of the compound is about 20 mg. In some embodiments, the dosage of the compound is about 40 mg. In some embodiments, the dosage of the compound is about 60 mg.

In some embodiments, the subject is a mammal (e.g., a human). In some embodiments, the subject is an adult (e.g., male or female). In some embodiments, the subject is a child.

In some embodiments, the method further comprises analyzing or receiving analysis of an EEG recording at least once prior to the end of treatment. In some embodiments, the dosage is adjusted (e.g., increased) based on analysis of an EEG recording.

In some embodiments, the method further comprises analyzing or receiving analysis of a blood sample from the subject at least once prior to the end of treatment. In some embodiments, the dosage is adjusted (e.g., increased) based on analysis of a blood sample.

In some embodiments, the compound is administered daily for one week at a dosage of 20 mg, then administered daily for a week at a dosage greater than 20 mg (e.g., 25 mg, 30 mg, 35 mg, 40 mg, 45 mg, or 50 mg). In some embodiments, the compound is administered daily for one week at a dosage of 20 mg, then administered daily for a week at a dosage of 40 mg, then administered daily for a week at a dosage greater than 40 mg (e.g., 45 mg, 50 mg, 55 mg, 60 mg, 65 mg, or 70 mg).

In some embodiments, the compound has selectivity for one T-type calcium channel over another. In some embodiments, the compound has selectivity for T-type calcium channel Cav3.2 compared with T-type calcium channel Cav3.1 or T-type calcium channel Cav3.3. In some embodiments, the compound has selectivity for T-type calcium channel Cav3.1 compared with T-type calcium channel Cav3.2 or T-type calcium channel Cav3.3. In some embodiments, the compound has selectivity for T-type calcium channel Cav3.3 compared with T-type calcium channel Cav3.1 or T-type calcium channel Cav3.2.

Other objects and advantages will become apparent to those skilled in the art from a consideration of the ensuing Brief Description of the Figures, Detailed Description, Examples, and Claims.

### BRIEF DESCRIPTION OF THE FIGURES

FIG. 1A is a graph of the effect of drug treatment on the percentage of time spent (seconds) in seizure activity in GAERS.

FIG. 1B is a graph of the average number of seizures per hour in GAERS.

FIG. 1C is a graph of the average seizure duration (seconds) in GAERS during the 120-min post-drug test period.

FIG. 2A is a graph of power (μV²) versus frequency (Hertz) for drugs compared to vehicle.

FIG. 2B is a graph of cycle frequency for drugs compared to vehicle.

FIG. 2C is a graph of power (μV²) versus frequency (Hertz) for drugs compared to vehicle.

FIG. 3A is a graph of inhibition (%) of hCav3.2 versus ethosuximide concentration (Molar) in an inactivated channel state FLIPR assay.

FIG. 3B is a graph of inhibition (%) of hCav3.2 versus valproate concentration (Molar) in an inactivated channel state FLIPR assay.

FIG. 3C is a graph of inhibition (%) of hCav3.2 versus Z941 concentration (Molar) in an inactivated channel state FLIPR assay.

FIG. 3D is a graph of inhibition (%) of hCav3.2 versus Z944 concentration (Molar) in an inactivated channel state FLIPR assay.

FIG. 4A is a trace of kinetics of recovery of rCav3.2 from inactivation, determined with a two-pulse protocol (P, 400 ms to −30 mV; P2, 50 ms to −30 mV) with a variable interpulse interval (5 to 5120 ms) applied every 20 s from a holding potential of −110 mV.

FIG. 4B is a graph of fraction of Cav3.2 current versus interval (seconds) for control compared to Z941.

FIG. 4C is a graph of fraction of Cav3.2 current versus interval (seconds) for control compared to Z944.

FIG. 5A is a graph of T-type inhibition (%) versus concentration of Z944 (Molar).

FIG. 5B is a graph of current density (pA/pF) versus Vₘ (mV) in absence or presence of 10 mM Z944.

FIG. 5C is a graph of current (pA) versus time (minutes) for T-type Ca2+ channel currents in response to 1 mM Z944 and wash off.

FIG. 6A is a graph of Vₘ (mV) versus time (milliseconds) for response to depolarizing current of (+80 pA).

FIG. 6B is a graph of Vₘ (mV) versus time (milliseconds) for response to depolarizing current of (+120 pA).

FIG. 6C is a graph of Vₘ (mV) versus time (milliseconds) for response to depolarizing current of (+100 pA).

FIG. 7A is a graph of percentage of time spent in seizures (%) for drug compared to vehicle in somatosensory cortex S2.

FIG. 7B is a graph of percentage of time spent in seizures (%) for drug compared to vehicle in thalamic reticular nucleus nRT.

FIG. 8A is a graph of seizure class for fully kindled animals for drugs compared to vehicle treatment.

FIG. 8B is a graph of primary discharge (seconds) for drugs compared to vehicle.

FIG. 8C is a graph of total seizure duration (seconds) for drugs compared to vehicle.

FIG. 8D is a graph of adverse effects for drugs compared to vehicle.

FIG. 9A is a graph of number of stimulations versus seizure class.

FIG. 9B is a graph of number of stimulations for fully kindled animals for drugs compared to vehicle.

FIG. 9C is a graph of seizure duration (seconds) versus number of stimulations.

FIG. 10 is a graph of adverse effects versus days after pump implantation.

FIG. 11 is a timeline of drug treatment protocol in the post status epilepticus model.

FIG. 12A is a heat map of average seizures per day during a 4 week treatment period post status epilepticus.

FIG. 12B is a graph of average seizures per day for drug compared to vehicle.

FIG. 12C is a graph of seizure class for drug compared to vehicle.

FIG. 12D is a graph of average seizure duration (minutes) for drug compared to vehicle.

FIG. 13A is a graph of average seizures per day for drug compared to vehicle.

FIG. 13B is a graph of seizure duration (minutes) for drug compared to vehicle.

FIG. 13C is a graph of seizure class for drug compared to vehicle.

FIG. 14A is a graph of sucrose preference (%) for drug compared to vehicle.

FIG. 14B is a graph of forced swim test total immobility time (seconds) for drug compared to vehicle.

FIG. 15 is a graph of time to find platform (seconds) versus session number for a cognition study in rats.

FIG. 16A is a graph of Cav 3.1 mRNA relative expression for drug compared to vehicle.

FIG. 16B is a graph of Cav 3.2 mRNA relative expression for drug compared to vehicle.

FIG. 16C is a graph of Cav 3.3 mRNA relative expression for drug compared to vehicle.

### DETAILED DESCRIPTION OF THE INVENTION

As generally described herein, the present invention provides compounds and compositions useful for preventing and/or treating epilepsy or epilepsy syndromes (e.g., absence seizures, juvenile myoclonic epilepsy, or a genetic epilepsy). Methods are also presented for treating mood disorders (e.g., depression, major depressive disorder, dysthymic disorder (e.g., mild depression), bipolar disorder (e.g., I and/or II), anxiety disorders (e.g., generalized anxiety disorder (GAD), social anxiety disorder), stress, post-traumatic stress disorder (PTSD), compulsive disorders (e.g., obsessive compulsive disorder (OCD)). Methods are also presented that are useful for modulating the function and enhancing the potency of a T-type calcium channel.

### Definitions

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, the term “refractory” refers to a disease, disorder, or condition that does not readily yield or respond to therapy or treatment, or is not controlled by a therapy or treatment. In some embodiments, a disease, disorder, or condition described herein is refractory (e.g., refractory epilepsy or refractory absence seizures) and does not respond to standard therapy or treatment.

As used herein, a “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

The terms “disease”, “disorder”, and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{th} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

The following terms are intended to have the meanings presented therewith below and are useful in understanding the description and intended scope of the present invention. When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range. For example “C₁₋₆alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₆₋₆alkyl.

“Alkyl” refers to a radical of a straight-chain or branched saturated hydrocarbon group, e.g., having 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂alkyl”). In some embodiments, an alkyl group has 1 carbon atom (“C₁ alkyl”). Examples of C₁₋₆ alkyl groups include methyl, ethyl, propyl, isopropyl, butyl, isobutyl, pentyl, hexyl, and the like.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

“Alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

“‘Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl.

“Hetero” when used to describe a compound or a group present on a compound means that one or more carbon atoms in the compound or group have been replaced by a nitrogen, oxygen, or sulfur heteroatom. Hetero may be applied to any of the alkyl groups described above such as alkyl, e.g., heteroalkyl; alkenyl, e.g., heteroalkenyl; alkynyl, e.g., heteroalkynyl; carbocyclyl, e.g., heterocyclyl; aryl, e.g., heteroaryl, and the like having from 1 to 5, and particularly from 1 to 3 heteroatoms.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Cyano” refers to the radical —CN.

“Halo” or “halogen” refers to fluoro (F), chloro (Cl), bromo (Br), and iodo (I). In certain embodiments, the halo group is either fluoro or chloro.

“Haloalkyl” refers to an alkyl group substituted with one or more halogen atoms.

“Nitro” refers to the radical —NO₂.

In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

A “counterion” or “anionic counterion” is a negatively charged group associated with a cationic quaternary amino group in order to maintain electronic neutrality. Exemplary counterions include halide ions (e.g., F⁻, Cr⁻, Br⁻, I⁻), NO₃⁻, ClO₄⁻, OH⁻, H₂PO₄⁻, HSO₄⁻, SO₄⁻² sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

These and other exemplary substituents are described in more detail in the Detailed Description, Examples, and Claims. The invention is not intended to be limited in any manner by the above exemplary listing of substituents.

### Compounds

The present invention features compounds and compositions thereof for the modulation of T-type calcium channels, as well as diseases, disorders, or conditions associated with aberrant function thereof (e.g., epilepsy or an epilepsy syndrome, e.g., absence seizures, juvenile myoclonic epilepsy, status epilepticus, or a genetic epilepsy).

In some embodiments, the compound is a compound of Formula (II):

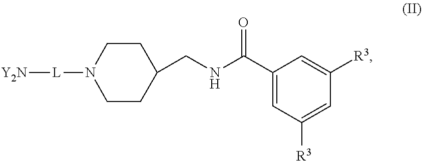

or a pharmaceutically acceptable salt thereof, wherein:

L is C(O)CH₂ or CH₂CH₂;

R³ is H, halo, CF₃, CH, OCH₃ or OCF₃;

each Y is independently H, SR″, SOR″, SO₂R″;

or each Y is an optionally substituted group selected from C₁-C₁₀ alkyl, C₂-C₁₀ alkenyl, C₂-C₁₀ alkynyl, C₂-C₁₀ heteroalkyl, C₂-C₁₀ heteroalkenyl, or C₂-C₁₀ heteroalkynyl;

or two Y may together form an optionally substituted heterocyclic ring (4-6 ring members); and

each R″ is independently H or an optionally substituted group selected from C₁-C₆ alkyl, C₂-C₆ alkenyl, C₂-C₆ alkynyl, C₂-C₆ heteroalkyl, C₂-C₆ heteroalkenyl, or C₂-C₆ heteroalkynyl; and

wherein the optional substituents on Y, R and R″ may be one or more halo, ═O, ═NOR′, CN, NO₂, CF₃, OCF₃, COOR′, CONR′₂, OR′, SR′, SOR′, SO₂R′, NR′₂, NR′(CO)R′, and NR′SO₂R′, C₁-C₆ alkyl, C₂-C₆ alkenyl, C₂-C₆ alkynyl, C₂-C₆ heteroalkyl, C₂-C₆ heteroalkenyl, or C₂-C₆ heteroalkynyl, as disclosed in U.S. Pat. No. 8,377,968, the entirety of which is incorporated herein by reference.

In some embodiments, each R³ is independently halo (e.g., fluoro or chloro). In some embodiments, one R³ is independently fluoro and the other R³ is independently chloro.

In some embodiments, L is C(O)CH₂.

In some embodiments, Y is C₁-C₁₀ alkyl. In some embodiments, Y is C₁-C₆ alkyl. In some embodiments, Y is C₁-C₄ alkyl. In some embodiments, Y is butyl.

In some embodiments, the compound is

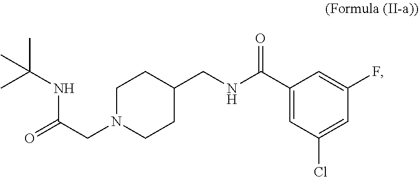 [SMILES: CC(C)(C)NC(=O)CN1CCC(CNC(=O)c2cc(F)cc(Cl)c2)CC1]

or a pharmaceutically acceptable salt thereof.

In some embodiments, the compound is

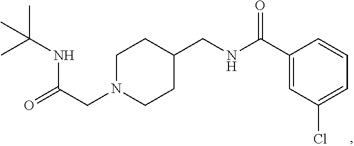 [SMILES: CC(C)(C)NC(=O)CN1CCC(CNC(=O)c2cccc(Cl)c2)CC1]

or a pharmaceutically acceptable salt thereof.

In some embodiments, the compound is a compound disclosed in U.S. Pat. No. 8,377,968.

In some embodiments, the compound is a compound disclosed in one or more of WO 2016/041892, WO 2015/186056, WO 2010/04857, WO 2010/046869, WO 2010/046855, WO 2009/130679, or WO 2008/132679, the contents of each of which are incorporated herein by reference in their entirety. In some embodiments, the compound is ACT 280778 or a pharmaceutically acceptable salt thereof.

In some embodiments, the compound is tylerdipine or a pharmaceutically acceptable salt thereof (e.g., tylerdipine hydrochloride).

In some embodiments, the compound is mibefradil or a pharmaceutically acceptable salt thereof. In some embodiments, the compound is

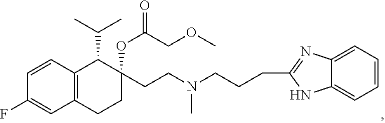 [SMILES: COCC(=O)O[C@]1(CCN(C)CCCc2nc3ccccc3[nH]2)CCc2cc(F)ccc2[C@@H]1C(C)C]

or a pharmaceutically acceptable salt thereof.

In some embodiments, the compound is a isotopically enriched isomer of mibefradil, or a pharmaceutically acceptable salt thereof. In some embodiments, the compound is C-10296 or C-10302, or a pharmaceutically acceptable salt thereof.

In some embodiments, the compound is benidipine, or a pharmaceutically acceptable salt thereof. In some embodiments, the compound is

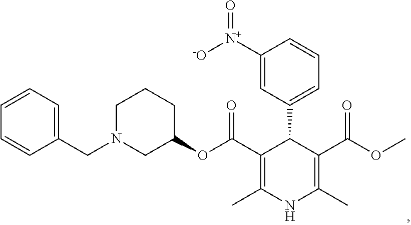

In some embodiments, the compound is a 3,4-dihydroquinazoline derivative, or a pharmaceutically acceptable salt thereof. In some embodiments, the compound is BK-10040. In some embodiments, the compound is

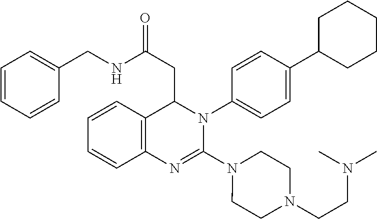 [SMILES: CN(C)CCN1CCN(C2=Nc3ccccc3C(CC(=O)NCc3ccccc3)N2c2ccc(C3CCCCC3)cc2)CC1]

or a pharmaceutically acceptable salt thereof.

In some embodiments, the compound is ML 218 (i.e., AFA-19), or a pharmaceutically acceptable salt thereof. In some embodiments, the compound is,

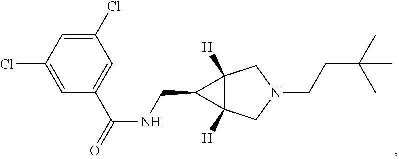 [SMILES: CC(C)(C)CCN1C[C@@H]2[C@@H](CNC(=O)c3cc(Cl)cc(Cl)c3)[C@@H]2C1]

or a pharmaceutically acceptable salt thereof.

In some embodiments, the compound is P-11520031, or a pharmaceutically acceptable salt thereof.

In some embodiments, the compound is TH-1177, or a pharmaceutically acceptable salt thereof. In some embodiments, the compound is

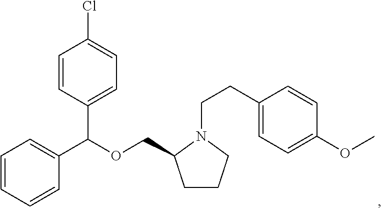 [SMILES: COc1ccc(CCN2CCC[C@H]2COC(c2ccccc2)c2ccc(Cl)cc2)cc1]

or a pharmaceutically acceptable salt thereof.

In some embodiments, the compound is DP-3005, or a pharmaceutically acceptable salt thereof.

In some embodiments, the compound is RQ-00311610, or a pharmaceutically acceptable salt thereof.

In some embodiments, the compound is A-1264087 or A-1315647, or a pharmaceutically acceptable salt thereof. In some embodiments, the compound is

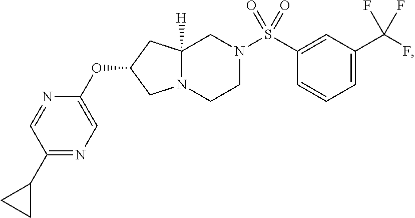 [SMILES: O=S(=O)(c1cccc(C(F)(F)F)c1)N1CCN2C[C@H](Oc3cnc(C4CC4)cn3)C[C@H]2C1]

or a pharmaceutically acceptable salt thereof.

In some embodiments, the compound is ABT-639, or a pharmaceutically acceptable salt thereof. In some embodiments, the compound is

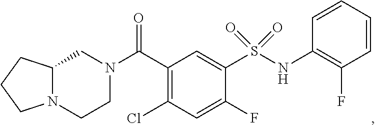 [SMILES: O=C(c1cc(S(=O)(=O)Nc2ccccc2F)c(F)cc1Cl)N1CCN2CCC[C@@H]2C1]

or a pharmaceutically acceptable salt thereof.

In some embodiments, the compound is a compound disclosed in WO 2010/083264, the entire contents of which are incorporated herein by reference their entirety. In some embodiments, the compound is

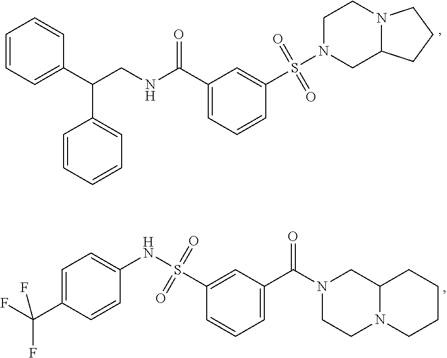 [SMILES: O=C(NCC(c1ccccc1)c1ccccc1)c1cccc(S(=O)(=O)N2CCN3CCCC3C2)c1 ; O=C(c1cccc(S(=O)(=O)Nc2ccc(C(F)(F)F)cc2)c1)N1CCN2CCCCC2C1]

or a pharmaceutically acceptable salt thereof.

In some embodiments, the compound is a 2-(1-alkylpiperidin-4-yl)-N-[(1R)-1-(4-fluorophenyl)-2-methylpropyl]acetamide derivative, or a pharmaceutically acceptable salt thereof. In some embodiments, the compound is N-[(1R)-1-(4-fluorophenyl)-2-methylpropyl]-2-(1-{2-[2-(2-methoxyethoxy)phenyl]ethyl}piperidin-4-yl)acetamide, or a pharmaceutically acceptable salt thereof. In some embodiments, the compound is

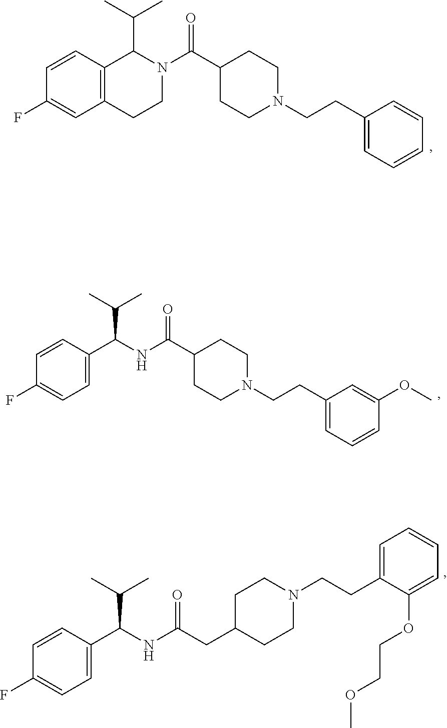 [SMILES: CC(C)C1c2ccc(F)cc2CCN1C(=O)C1CCN(CCc2ccccc2)CC1 ; COc1cccc(CCN2CCC(C(=O)N[C@@H](c3ccc(F)cc3)C(C)C)CC2)c1 ; COCCOc1ccccc1CCN1CCC(CC(=O)N[C@@H](c2ccc(F)cc2)C(C)C)CC1]

or a pharmaceutically acceptable salt thereof.

In some embodiments, the compound is a 1-alkyl-N-[2-ethyl-2-(4-fluorophenyl)butylpiperidine-4-carboxamide derivative, or a pharmaceutically acceptable salt thereof. In some embodiments, the compound is N-[(1R)-1-(4-fluorophenyl)-2-methylpropyl]-1-[2-(3-methoxyphenyl)ethyl]piperidine-4-carboxamide, or a pharmaceutically acceptable salt thereof.

In some embodiments, the compound is a 1-isopropyl-1,2,3,4-tetrahydroisoquinoline derivative, or a pharmaceutically acceptable salt thereof. In some embodiments, the compound is 6-fluoro-1-isopropyl-2-{[1-(2-phenylethyl)piperidin-4-yl)carbonyl-1,2,3,4-tetrahydroisoquinoline, or a pharmaceutically acceptable salt thereof.

In some embodiments, the compound is HYP-10, or a pharmaceutically acceptable salt thereof. In some embodiments, the compound is

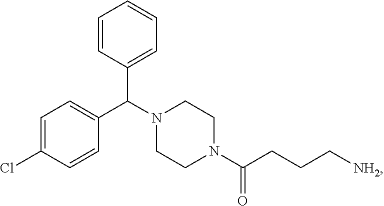 [SMILES: NCCCC(=O)N1CCN(C(c2ccccc2)c2ccc(Cl)cc2)CC1]

or a pharmaceutically acceptable salt thereof.

In some embodiments, the compound is a compound disclosed in WO 2009/054984, the contents of which are incorporated herein by reference in its entirety. In some embodiments, the compound is TTA-A8, TTA-A2, or TTA-P2, or a pharmaceutically acceptable salt thereof. In some embodiments, the compound is

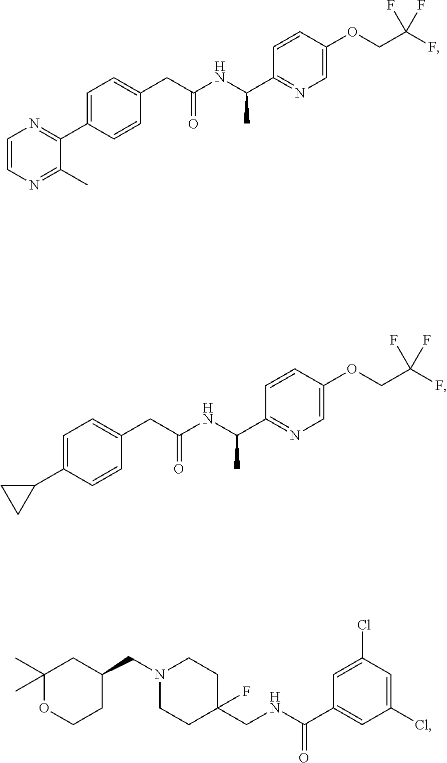 [SMILES: Cc1nccnc1-c1ccc(CC(=O)N[C@H](C)c2ccc(OCC(F)(F)F)cn2)cc1 ; C[C@@H](NC(=O)Cc1ccc(C2CC2)cc1)c1ccc(OCC(F)(F)F)cn1 ; CC1(C)C[C@@H](CN2CCC(F)(CNC(=O)c3cc(Cl)cc(Cl)c3)CC2)CCO1]

or a pharmaceutically acceptable salt thereof.

In some embodiments, the compound is a compound disclosed in WO 2004/035000, WO93/04047, WO 2006/098969, WO 2009/009015, WO 2007/002361, WO 2007/002884, WO 2007/120729, WO 2009/054983, WO 2009/054982, or WO 2009/054984, the contents of each of which are incorporated herein by reference in their entirety. In some embodiments, the compound is TTA-Q3, TTA-Q4, TTA-P1, TTA-P2, TTA-A1, TTA-A2, or TTA-A8. In some embodiments, the compound is

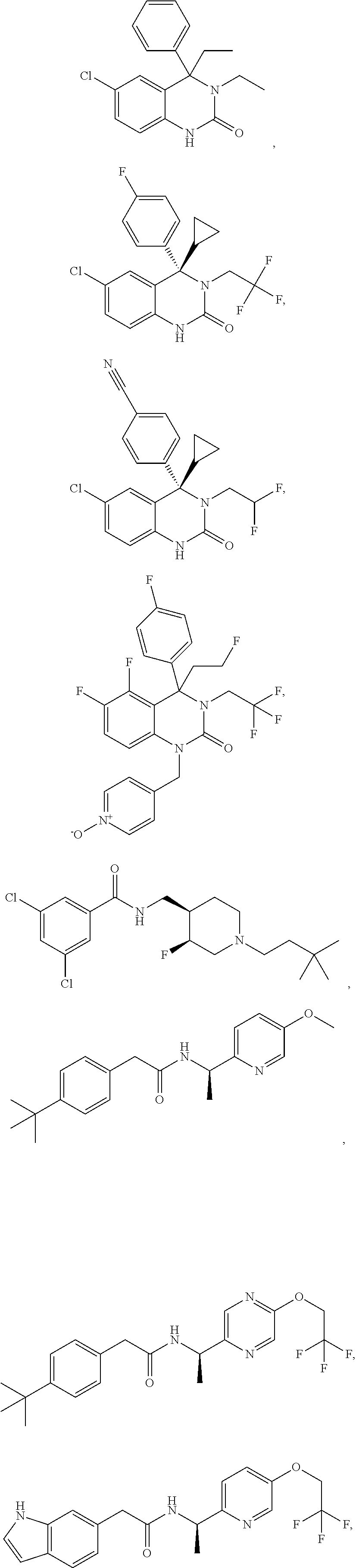

or a pharmaceutically acceptable salt thereof.

In some embodiments, the compound is a compound disclosed in WO 2005/007124, WO 2005/009392, or US-2005-245535, the contents of each of which are incorporated herein by reference in their entirety. In some embodiments, the compound is

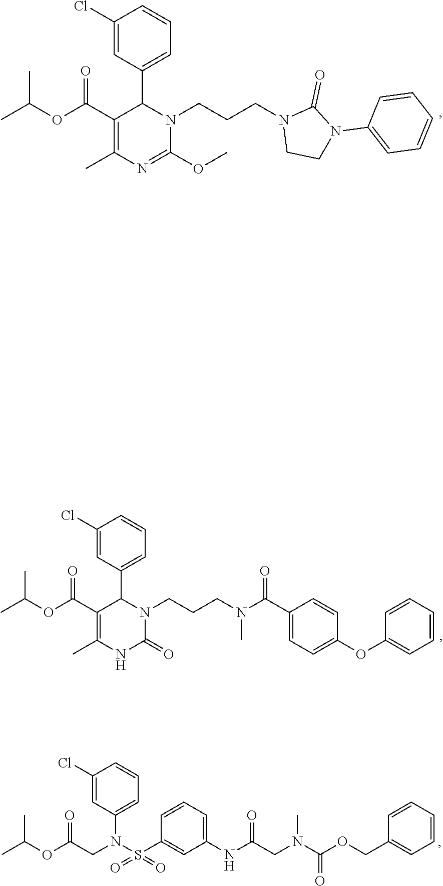 [SMILES: COC1=NC(C)=C(C(=O)OC(C)C)C(c2cccc(Cl)c2)N1CCCN1CCN(c2ccccc2)C1=O ; CC1=C(C(=O)OC(C)C)C(c2cccc(Cl)c2)N(CCCN(C)C(=O)c2ccc(Oc3ccccc3)cc2)C(=O)N1 ; CC(C)OC(=O)CN(c1cccc(Cl)c1)S(=O)(=O)c1cccc(NC(=O)CN(C)C(=O)OCc2ccccc2)c1]

or a pharmaceutically acceptable salt thereof.

In some embodiments, the compound is a compound disclosed in US-2009-0270413, WO 2008/110008, WO 2009/146539, US-2008-0280900, the contents of each of which are incorporated herein by reference in their entirety. In some embodiments, the compound is

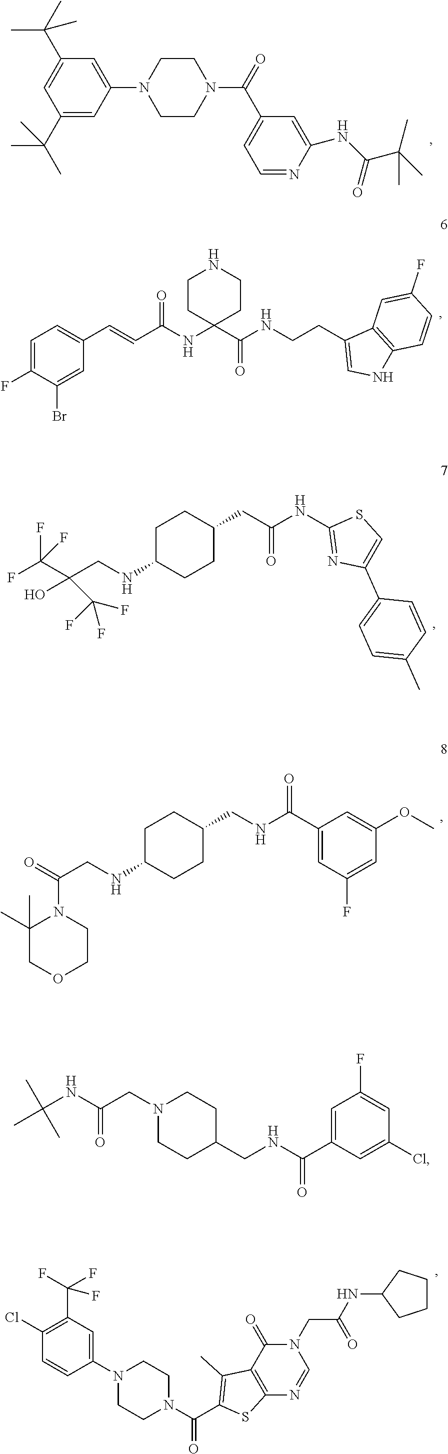

or a pharmaceutically acceptable salt thereof.

In some embodiments, the compound is a compound disclosed in WO 2007/073497 or WO 20067/075852, the contents of each of which are incorporated herein by reference in their entirety. In some embodiments, the compound is

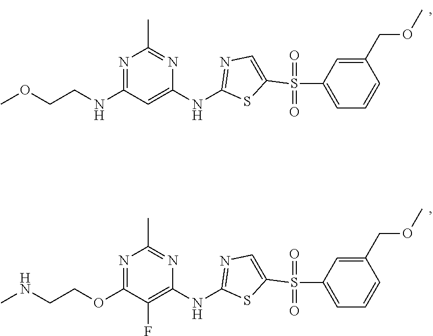

or a pharmaceutically acceptable salt thereof.

In some embodiments, the compound is a compound disclosed in WO 2008/033456, WO 2008/033456, WO 2008/033460, WO 2008/0334664, or WO 2008/033465, the contents of each of which are incorporated herein by reference in their entirety. In some embodiments, the compound is

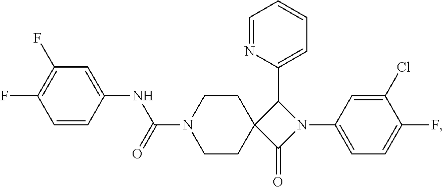 [SMILES: O=C(Nc1ccc(F)c(F)c1)N1CCC2(CC1)C(=O)N(c1ccc(F)c(Cl)c1)C2c1ccccn1]

or a pharmaceutically acceptable salt thereof.

In some embodiments, the compound is KST-5468 or KST-005, or a pharmaceutically acceptable salt thereof. In some embodiments, the compound is

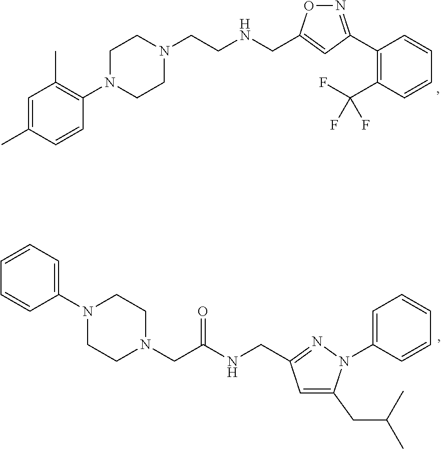 [SMILES: Cc1ccc(N2CCN(CCNCc3cc(-c4ccccc4C(F)(F)F)no3)CC2)c(C)c1 ; CC(C)Cc1cc(CNC(=O)CN2CCN(c3ccccc3)CC2)nn1-c1ccccc1]

or a pharmaceutically acceptable salt thereof.

In some embodiments, the compound is VMD-3816 or VMD-3222, or a pharmaceutically acceptable salt thereof.

In some embodiments, the compound is KYS-05047, KYS-5090, BK-10007S, or BK-10008S, or a pharmaceutically acceptable salt thereof. In some embodiments, the compound is

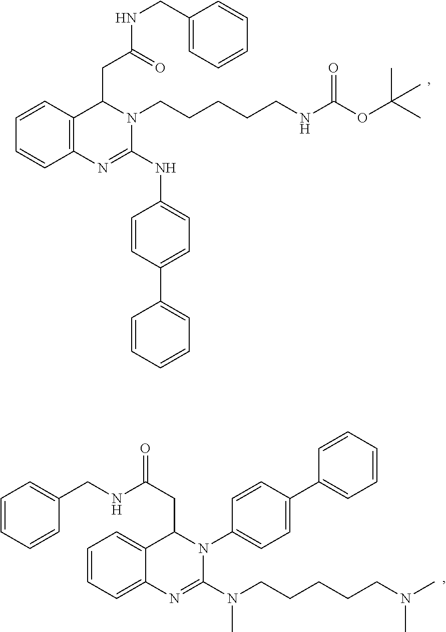 [SMILES: CC(C)(C)OC(=O)NCCCCCN1C(Nc2ccc(-c3ccccc3)cc2)=Nc2ccccc2C1CC(=O)NCc1ccccc1 ; CN(C)CCCCCN(C)C1=Nc2ccccc2C(CC(=O)NCc2ccccc2)N1c1ccc(-c2ccccc2)cc1]

or a pharmaceutically acceptable salt thereof.

In some embodiments, the compound is a compound disclosed in U.S. Pat. No. 7,319,098, US-2010-0004286, US-2010-094006, US-2010-0056545, EP1568695, E01757590, KR754325, KR758317, KR-2009-044924, WO 2008/007835, WO 2008/018655, or WO 2009/035307, the contents of each of which are incorporated herein by reference in their entirety. In some embodiments, the compound is

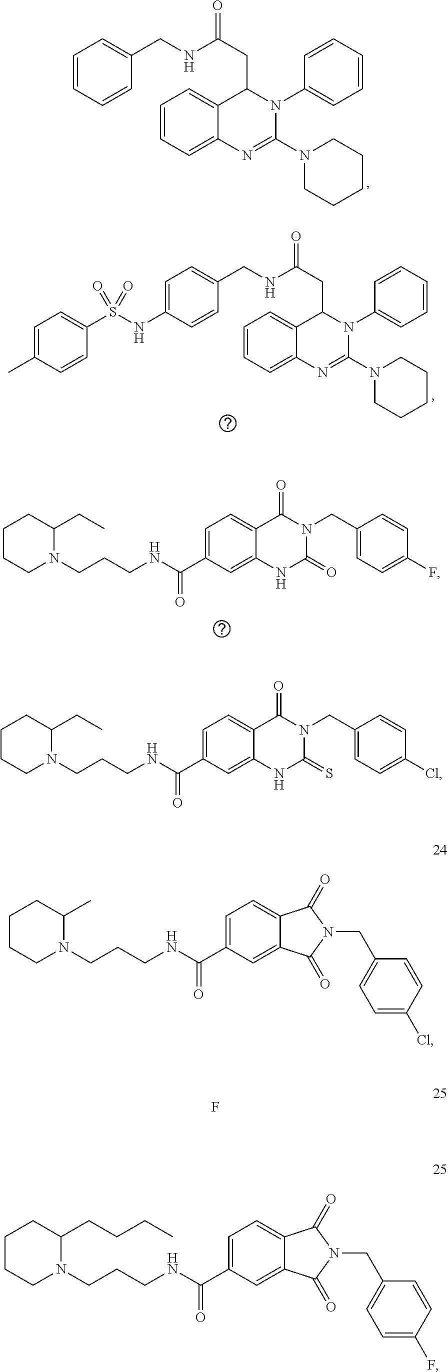

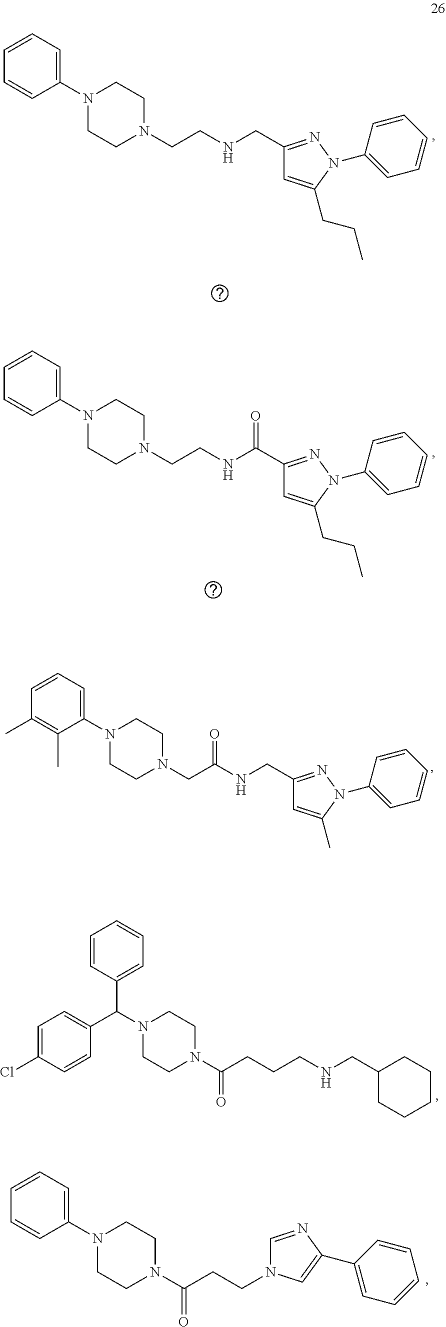 [SMILES: CCCc1cc(CNCCN2CCN(c3ccccc3)CC2)nn1-c1ccccc1 ; CCCc1cc(C(=O)NCCN2CCN(c3ccccc3)CC2)nn1-c1ccccc1 ; Cc1cccc(N2CCN(CC(=O)NCc3cc(C)n(-c4ccccc4)n3)CC2)c1C ; O=C(CCCNCC1CCCCC1)N1CCN(C(c2ccccc2)c2ccc(Cl)cc2)CC1 ; O=C(CCn1cnc(-c2ccccc2)c1)N1CCN(c2ccccc2)CC1]

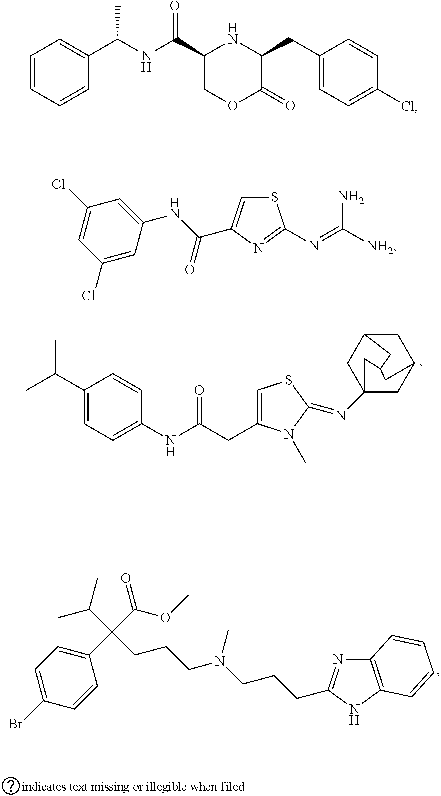 [SMILES: C[C@H](NC(=O)[C@@H]1COC(=O)[C@H](Cc2ccc(Cl)cc2)N1)c1ccccc1 ; NC(N)=Nc1nc(C(=O)Nc2cc(Cl)cc(Cl)c2)cs1 ; CC(C)c1ccc(NC(=O)Cc2cs/c(=N\C34CC5CC(CC(C5)C3)C4)n2C)cc1 ; COC(=O)C(CCCN(C)CCCc1nc2ccccc2[nH]1)(c1ccc(Br)cc1)C(C)C]

or a pharmaceutically acceptable salt thereof.

In some embodiments, the compound is diltiazem or a derivative thereof, or a pharmaceutically acceptable salt thereof. In some embodiments, the compound is a compound disclosed in WO 2005/009392 or WO 2005/007124, the contents of each of which are incorporated herein by reference in their entirety. In some embodiments, the compound is

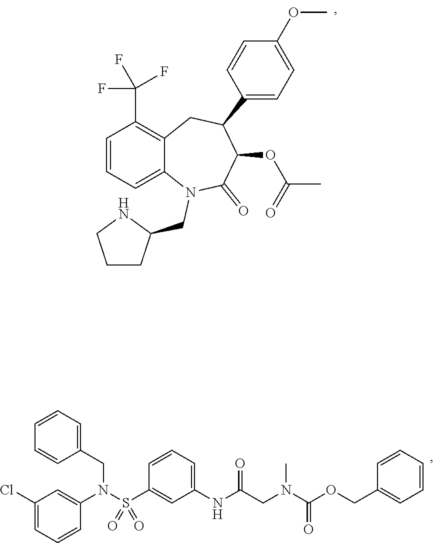 [SMILES: COc1ccc([C@@H]2Cc3c(cccc3C(F)(F)F)N(C[C@H]3CCCN3)C(=O)[C@@H]2OC(C)=O)cc1 ; CN(CC(=O)Nc1cccc(S(=O)(=O)N(Cc2ccccc2)c2cccc(Cl)c2)c1)C(=O)OCc1ccccc1]

or a pharmaceutically acceptable salt thereof.

In some embodiments, the compound is a compound disclosed in WO 2006/023881, WO 2006/023883, or WO 2005/077082, the contents of each of which are incorporated herein by reference in their entirety. In some embodiments, the compound is

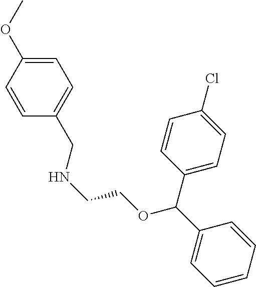

or a pharmaceutically acceptable salt thereof.

In some embodiments, the compound is a compound disclosed in WO 2008/050200, WO 2008/117148, or WO 2009/056934, the contents of each of which are incorporated herein by reference in their entirety. In some embodiments, the compound is

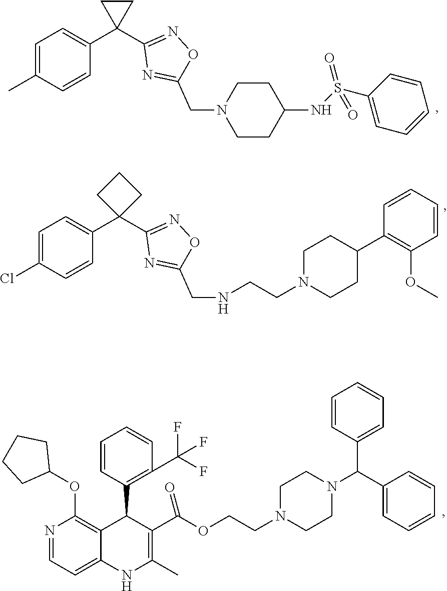 [SMILES: Cc1ccc(C2(c3noc(CN4CCC(NS(=O)(=O)c5ccccc5)CC4)n3)CC2)cc1 ; COc1ccccc1C1CCN(CCNCc2nc(C3(c4ccc(Cl)cc4)CCC3)no2)CC1 ; CC1=C(C(=O)OCCN2CCN(C(c3ccccc3)c3ccccc3)CC2)[C@H](c2ccccc2C(F)(F)F)c2c(ccnc2OC2CCCC2)N1]

or a pharmaceutically acceptable salt thereof.

In some embodiments, the compound is a compound disclosed in Giordanetto, F. et al, Exp Opin Ther Patents (2011) 21:185-101; Xiang, Z. et al, ACS Chem Neurosci(2011) 2:730-742; Bourinet, E. et al, Pain (2016) 157:S15-S22; Cheong E. and Shin, H. (2014) 466:719-734; Fritch, P. C. and Krajewski, J. (2010) Bioorg Med Chem Lett 20:6375-6378; Casillas-Espinosa, P. M. et al, PLOS One (2015) 10(8):e0130012; or Tringham E. et al, Sci Transl Med (2012) 4(121) 121ra19, the contents of each of which are incorporated herein by reference in their entirety.

### Methods of Treatment

Described herein are compounds and compositions thereof and their use to treat a disease, disorder, or condition relating to aberrant function of T-type calcium channel. In some embodiments, a compound provided by the present invention is effective in the treatment of an epilepsy or epilepsy syndrome, e.g., absence seizures, juvenile myoclonic epilepsy, status epilepticus, or a genetic epilepsy. Compounds of the invention may also modulate all T-type calcium channels, or may be specific to only one or a plurality of T-type calcium channels, e.g., Cav3.1, Cav3.2, and/or Cav3.3.

In typical embodiments, the present invention is intended to encompass the compounds disclosed herein, and the pharmaceutically acceptable salts, tautomeric forms, polymorphs, and prodrugs of such compounds. In some embodiments, the present invention includes a pharmaceutically acceptable addition salt, a hydrate of an addition salt, a tautomeric form, a polymorph, an enantiomer, a mixture of enantiomers, a stereoisomer or mixture of stereoisomers (pure or as a racemic or non-racemic mixture) of a compound described herein.

### Epilepsy and Epilepsy Syndromes

The compounds described herein are useful in the treatment of epilepsy and epilepsy syndromes. Epilepsy is a CNS disorder in which nerve cell activity in the brain becomes disrupted, causing seizures or periods of unusual behavior, sensations and sometimes loss of consciousness. Seizure symptoms will vary widely, from a simple blank stare for a few seconds to repeated twitching of their arms or legs during a seizure.

Epilepsy may involve a generalized seizure or a partial or focal seizure. All areas of the brain are involved in a generalized seizure. A person experiencing a generalized seizure may cry out or make some sound, stiffen for several seconds to a minute a then have rhythmic movements of the arms and legs. The eyes are generally open, the person may appear not to be breathing and actually turn blue. The return to consciousness is gradual and the person maybe confused from minutes to hours. There are six main types of generalized seizures: tonic-clonic, tonic, clonic, myoclonic, absence, and atonic seizures. In a partial or focal seizure, only part of the brain is involved, so only part of the body is affected. Depending on the part of the brain having abnormal electrical activity, symptoms may vary.

Epilepsy, as described herein, includes a generalized, partial, complex partial, tonic clonic, clonic, tonic, refractory seizures, status epilepticus, absence seizures, febrile seizures, or temporal lobe epilepsy.

The compounds described herein may also be useful in the treatment of epilepsy syndromes. Severe syndromes with diffuse brain dysfunction caused, at least partly, by some aspect of epilepsy, are also referred to as epileptic encephalopathies. These are associated with frequent seizures that are resistant to treatment and severe cognitive dysfunction, for instance West syndrome.

In some embodiments, the epilepsy syndrome comprises epileptic encephalopathy, Dravet syndrome, Angelman syndrome, CDKL5 disorder, frontal lobe epilepsy, infantile spasms, West's syndrome, Juvenile Myoclonic Epilepsy, Landau-Kleffner syndrome, Lennox-Gastaut syndrome, Ohtahara syndrome, PCDH19 epilepsy, or Gluti deficiency.

### Absence Seizures

Absence seizures are one of the most common seizure types in patients with idiopathic generalised epilepsy (IGE) (Berg et al., Epilepsia 2000). Absence seizures are relatively brief, non-convulsive seizures characterised by abrupt onset of loss of awareness and responsiveness, usually lasting between 10-30 seconds in duration, with a rapid return to normal consciousness without post-ictal confusion. The seizures are characterised on an accompanying EEG recording by the abrupt onset and offset of generalised 1-6 Hz (e.g., 3 Hz) spike and wave discharges. Absence seizure often occur multiple times per day, interrupt learning and psychosocial functioning, and present a risk of injury because of the frequent episodes of loss of awareness. Typically, absence seizures begin in early childhood and remit by teenage years. However, in a minority of patients they persist into adulthood where they are often drug resistant, and may be accompanied by other seizure types such as generalised tonic-clonic seizures. In these adult patients, the absence seizures are usually highly disabling, in particular by disqualifying the sufferer from obtaining a motor vehicle license or pursuing occupations and hobbies in which the seizures-associated periods of loss of awareness pose a safety risk, and are associated with significant psychosocial disabilities (Wirrell et al., 1997).

While there is a common perception that absence seizures are relatively “easy” to treat, a randomised control trial in patients with childhood absence epilepsy showed that even the most effective anti-epileptic drugs, ethosuximide and valproate, only completely controlled the seizures in 53% and 58% of patients respectively at 16 weeks as assessed by video-EEG recordings (Glauser et al., 2010), and 45% and 44% respectively at 12 months (Glauser et al., 2013). Lamotrigine, the other AED commonly used to treat absence seizures, only controlled the seizures in 29% of patients at 16 weeks, and 21% of patients at 12 months. Furthermore, both ethosuximide and valproate are commonly associated with intolerable side effects (occurring in 24% of patients treated with either of these drugs) (Glauser et al., 2101), and the latter is now generally considered to be contraindicated in girls and women of childbearing potential. Other treatment options for absence seizures are limited, with only benzodiazepines having established efficacy—and these are commonly poorly tolerated due to sedative and cognitive side effects. Absence seizures persisting into adult life are particularly difficult to treat, with patients often being treated with multiple drugs resulting in significant side-effects without attaining seizure control.

There is abundant evidence that low threshold (T-type) calcium channels play a critical role in the generation and maintenance of absence seizures, being a key component of the oscillatory burst firing that occurs in thalamocortical neurones during absence seizures (Pinault and O'Brien, 1997). In some embodiments, the present invention features a method for treating absence seizures with a compound described herein. In some embodiments, the absence seizures are refractory absence seizures. In some embodiments, the absence seizures are refractory to an anti-epileptic drug (e.g., ethosuximide, valproic acid, or lamotrigine).

In some embodiments, the subject has epilepsy. In some embodiments, the absence seizures are atypical absence seizures. In some embodiments, the absence seizures comprise adult absence seizures, juvenile absence seizures, or childhood absence seizures.

In some embodiments, the methods described herein further comprise identifying a subject having absence seizures. In some embodiments, the subject has a mutation in one or both of the T-type calcium channel genes CACNA1H and CACNA1G.

### Genetic Epilepsies

In some embodiments, the epilepsy or epilepsy syndrome is a genetic epilepsy or a genetic epilepsy syndrome. In some embodiments, epilepsy or an epilepsy syndrome comprises epileptic encephalopathy, epileptic encephalopathy with SCN1A, SCN2A, SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, and KCNT1 epileptic encephalopathy.

In some embodiments, the methods described herein further comprise identifying a subject having epilepsy or an epilepsy syndrome (e.g., epileptic encephalopathy, epileptic encephalopathy with SCN1A, SCN2A, SCN8A mutations, early infantile epileptic encephalopathy, Dravet syndrome, Dravet syndrome with SCN1A mutation, generalized Epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, and KCNT1 epileptic encephalopathy) prior to administration of a compound described herein.

In one aspect, the present invention features a method of treating epilepsy or an epilepsy syndrome (e.g., epileptic encephalopathy, epileptic encephalopathy with SCN1A, SCN2A, SCN8A mutations, early infantile epileptic encephalopathy, Dravet syndrome, Dravet syndrome with SCN1A mutation, generalized Epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, and KCNT1 epileptic encephalopathy) comprising administering to a subject in need thereof a compound described herein.

A compound of the present invention may also be used to treat an epileptic encephalopathy, wherein the subject has a mutation in one or more of ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNAO1, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCN1A, SCN1B, SCN2A, SCN8A, SCN9A, SIAT9, SIK1, SLC13A5, SLC25A22, SLC2A1, SLC35A2, SLC6A, SNIP1, SPTAN1, SRPX2, ST3GAL3, STRADA, STX1B, STXBP1, SYN1, SYNGAP1, SZT2, TBC1D24, and WWOX.

In some embodiments, the methods described herein further comprise identifying a subject having a mutation in one or more of ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNAO1, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCN1A, SCN1B, SCN2A, SCN8A, SCN9A, SIAT9, SIK1, SLC13A5, SLC25A22, SLC2A1, SLC35A2, SLC6A1, SNIP1, SPTAN1, SRPX2, ST3GAL3, STRADA, STX1B, STXBP1, SYN1, SYNGAP1, SZT2, TBC1D24, and WWOX prior to administration of a compound described herein.

### Mood Disorders

Also provided herein are methods for treating a mood disorder, for example clinical depression, postnatal depression or postpartum depression, perinatal depression, atypical depression, melancholic depression, psychotic major depression, cataonic depression, seasonal affective disorder, dysthymia, double depression, depressive personality disorder, recurrent brief depression, minor depressive disorder, bipolar disorder or manic depressive disorder, depression caused by chronic medical conditions, treatment-resistant depression, refractory depression, suicidality, suicidal ideation, or suicidal behavior. In some embodiments, the method described herein provides therapeutic effect to a subject suffering from depression (e.g., moderate or severe depression). In some embodiments, the mood disorder is associated with a disease or disorder described herein (e.g., neuroendocrine diseases and disorders, neurodegenerative diseases and disorders (e.g., epilepsy), movement disorders, tremor (e.g., Parkinson's Disease), women's health disorders or conditions).

Clinical depression is also known as major depression, major depressive disorder (MDD), severe depression, unipolar depression, unipolar disorder, and recurrent depression, and refers to a mental disorder characterized by pervasive and persistent low mood that is accompanied by low self-esteem and loss of interest or pleasure in normally enjoyable activities. Some people with clinical depression have trouble sleeping, lose weight, and generally feel agitated and irritable. Clinical depression affects how an individual feels, thinks, and behaves and may lead to a variety of emotional and physical problems. Individuals with clinical depression may have trouble doing day-to-day activities and make an individual feel as if life is not worth living.

Peripartum depression refers to depression in pregnancy. Symptoms include irritability, crying, feeling restless, trouble sleeping, extreme exhaustion (emotional and/or physical), changes in appetite, difficulty focusing, increased anxiety and/or worry, disconnected feeling from baby and/or fetus, and losing interest in formerly pleasurable activities.

Postnatal depression (PND) is also referred to as postpartum depression (PPD), and refers to a type of clinical depression that affects women after childbirth. Symptoms can include sadness, fatigue, changes in sleeping and eating habits, reduced sexual desire, crying episodes, anxiety, and irritability. In some embodiments, the PND is a treatment-resistant depression (e.g., a treatment-resistant depression as described herein). In some embodiments, the PND is refractory depression (e.g., a refractory depression as described herein).

In some embodiments, a subject having PND also experienced depression, or a symptom of depression during pregnancy. This depression is referred to herein as) perinatal depression. In an embodiment, a subject experiencing perinatal depression is at increased risk of experiencing PND.

Atypical depression (AD) is characterized by mood reactivity (e.g., paradoxical anhedonia) and positivity, significant weight gain or increased appetite. Patients suffering from AD also may have excessive sleep or somnolence (hypersomnia), a sensation of limb heaviness, and significant social impairment as a consequence of hypersensitivity to perceived interpersonal rejection.

Melancholic depression is characterized by loss of pleasure (anhedonia) in most or all activities, failures to react to pleasurable stimuli, depressed mood more pronounced than that of grief or loss, excessive weight loss, or excessive guilt.

Psychotic major depression (PMD) or psychotic depression refers to a major depressive episode, in particular of melancholic nature, where the individual experiences psychotic symptoms such as delusions and hallucinations.

Catatonic depression refers to major depression involving disturbances of motor behavior and other symptoms. An individual may become mute and stuporose, and either is immobile or exhibits purposeless or bizarre movements.

Seasonal affective disorder (SAD) refers to a type of seasonal depression wherein an individual has seasonal patterns of depressive episodes coming on in the fall or winter.

Dysthymia refers to a condition related to unipolar depression, where the same physical and cognitive problems are evident. They are not as severe and tend to last longer (e.g., at least 2 years).

Double depression refers to fairly depressed mood (dysthymia) that lasts for at least 2 years and is punctuated by periods of major depression.

Depressive Personality Disorder (DPD) refers to a personality disorder with depressive features.

Recurrent Brief Depression (RBD) refers to a condition in which individuals have depressive episodes about once per month, each episode lasting 2 weeks or less and typically less than 2-3 days.

Minor depressive disorder or minor depression refers to a depression in which at least 2 symptoms are present for 2 weeks.

Bipolar disorder or manic depressive disorder causes extreme mood swings that include emotional highs (mania or hypomania) and lows (depression). During periods of mania the individual may feel or act abnormally happy, energetic, or irritable. They often make poorly thought out decisions with little regard to the consequences. The need for sleep is usually reduced. During periods of depression there may be crying, poor eye contact with others, and a negative outlook on life. The risk of suicide among those with the disorder is high at greater than 6% over 20 years, while self harm occurs in 30-40%. Other mental health issues such as anxiety disorder and substance use disorder are commonly associated with bipolar disorder.

Depression caused by chronic medical conditions refers to depression caused by chronic medical conditions such as cancer or chronic pain, chemotherapy, chronic stress.

Treatment-resistant depression refers to a condition where the individuals have been treated for depression, but the symptoms do not improve. For example, antidepressants or physchological counseling (psychotherapy) do not ease depression symptoms for individuals with treatment-resistant depression. In some cases, individuals with treatment-resistant depression improve symptoms, but come back. Refractory depression occurs in patients suffering from depression who are resistant to standard pharmacological treatments, including tricyclic antidepressants, MAOIs, SSRIs, and double and triple uptake inhibitors and/or anxiolytic drugs, as well as non-pharmacological treatments (e.g., psychotherapy, electroconvulsive therapy, vagus nerve stimulation and/or transcranial magnetic stimulation).

Post-surgical depression refers to feelings of depression that follow a surgical procedure (e.g., as a result of having to confront one's mortality). For example, individuals may feel sadness or empty mood persistently, a loss of pleasure or interest in hobbies and activities normally enjoyed, or a persistent felling of worthlessness or hopelessness.

Mood disorder associated with conditions or disorders of women's health refers to mood disorders (e.g., depression) associated with (e.g., resulting from) a condition or disorder of women's health (e.g., as described herein).

Suicidality, suicidal ideation, suicidal behavior refers to the tendency of an individual to commit suicide. Suicidal ideation concerns thoughts about or an unusual preoccupation with suicide. The range of suicidal ideation varies greatly, from e.g., fleeting thoughts to extensive thoughts, detailed planning, role playing, incomplete attempts. Symptoms include talking about suicide, getting the means to commit suicide, withdrawing from social contact, being preoccupied with death, feeling trapped or hopeless about a situation, increasing use of alcohol or drugs, doing risky or self-destructive things, saying goodbye to people as if they won't be seen again.

Symptoms of depression include persistent anxious or sad feelings, feelings of helplessness, hopelessness, pessimism, worthlessness, low energy, restlessness, difficulty sleeping, sleeplessness, irritability, fatigue, motor challenges, loss of interest in pleasurable activities or hobbies, loss of concentration, loss of energy, poor self-esteem, absence of positive thoughts or plans, excessive sleeping, overeating, appetite loss, insomnia, self-harm, thoughts of suicide, and suicide attempts. The presence, severity, frequency, and duration of symptoms may vary on a case to case basis. Symptoms of depression, and relief of the same, may be ascertained by a physician or psychologist (e.g., by a mental state examination).

In some embodiments, the mood disorder is selected from depression, major depressive disorder, bipolar disorder, dysthymic disorder, anxiety disorders, stress, post-traumatic stress disorder, bipolar disorder, and compulsive disorders.

In some embodiments, the method comprises monitoring a subject with a known depression scale, e.g., the Hamilton Depression (HAM-D) scale, the Clinical Global Impression-Improvement Scale (CGI), and the Montgomery-Asberg Depression Rating Scale (MADRS). In some embodiments, a therapeutic effect can be determined by reduction in Hamilton Depression (HAM-D) total score exhibited by the subject. Reduction in the HAM-D total score can happen within 4, 3, 2, or 1 days; or 96, 84, 72, 60, 48, 24, 20, 16, 12, 10, 8 hours or less. The therapeutic effect can be assessed across a specified treatment period. For example, the therapeutic effect can be determined by a decrease from baseline in HAM-D total score after administering a compound described herein (e.g., 12, 24, or 48 hours after administration; or 24, 48, 72, or 96 hours or more; or 1 day, 2 days, 14 days, 21 days, or 28 days; or 1 week, 2 weeks, 3 weeks, or 4 weeks; or 1 month, 2 months, 6 months, or 10 months; or 1 year, 2 years, or for life).

In some embodiments, the subject has a mild depressive disorder, e.g., mild major depressive disorder. In some embodiments, the subject has a moderate depressive disorder, e.g., moderate major depressive disorder. In some embodiments, the subject has a severe depressive disorder, e.g., severe major depressive disorder. In some embodiments, the subject has a very severe depressive disorder, e.g., very severe major depressive disorder. In some embodiments, the baseline HAM-D total score of the subject (i.e., prior to treatment with a compound described herein), is at least 24. In some embodiments, the baseline HAM-D total score of the subject is at least 18. In some embodiments, the baseline HAM-D total score of the subject is between and including 14 and 18. In some embodiments, the baseline HAM-D total score of the subject is between and including 19 and 22. In some embodiments, the HAM-D total score of the subject before treatment with a compound described herein is greater than or equal to 23. In some embodiments, the baseline score is at least 10, 15, or 20. In some embodiments, the HAM-D total score of the subject after treatment with a compound described herein is about 0 to 10 (e.g., less than 10; 0 to 10, 0 to 6, 0 to 4, 0 to 3, 0 to 2, or 1.8). In some embodiments, the HAM-D total score after treatment with a compound described herein is less than 10, 7, 5, or 3. In some embodiments, the decrease in HAM-D total score is from a baseline score of about 20 to 30 (e.g., 22 to 28, 23 to 27, 24 to 27, 25 to 27, 26 to 27) to a HAM-D total score at about 0 to 10 (e.g., less than 10; 0 to 10, 0 to 6, 0 to 4, 0 to 3, 0 to 2, or 1.8) after treatment with a compound described herein. In some embodiments, the decrease in the baseline HAM-D total score to HAM-D total score after treatment with a compound described herein is at least 1, 2, 3, 4, 5, 7, 10, 25, 40, 50, or 100 fold). In some embodiments, the percentage decrease in the baseline HAM-D total score to HAM-D total score after treatment with a compound described herein is at least 50% (e.g., 60%, 70%, 80%, or 90%). In some embodiments, the therapeutic effect is measured as a decrease in the HAM-D total score after treatment with a compound described herein relative to the baseline HAM-D total score (e.g., 12, 24, 48 hours after administration; or 24, 48, 72, 96 hours or more; or 1 day, 2 days, 14 days, or more) is at least 10, 15, or 20 points.

In some embodiments, the method of treating a depressive disorder, e.g., major depressive disorder provides a therapeutic effect (e.g., as measured by reduction in Hamilton Depression Score (HAM-D)) within 14, 10, 4, 3, 2, or 1 days, or 24, 20, 16, 12, 10, or 8 hours or less. In some embodiments, the method of treating the depressive disorder, e.g., major depressive disorder, provides a therapeutic effect (e.g., as determined by a statistically significant reduction in HAM-D total score) within the first or second day of the treatment with a compound described herein. In some embodiments, the method of treating the depressive disorder, e.g., major depressive disorder, provides a therapeutic effect (e.g., as determined by a statistically significant reduction in HAM-D total score) within less than or equal to 14 days since the beginning of the treatment with a compound described herein. In some embodiments, the method of treating the depressive disorder, e.g., major depressive disorder, provides a therapeutic effect (e.g., as determined by a statistically significant reduction in HAM-D total score) within less than or equal to 21 days since the beginning of the treatment with a compound described herein. In some embodiments, the method of treating the depressive disorder, e.g., major depressive disorder, provides a therapeutic effect (e.g., as determined by a statistically significant reduction in HAM-D total score) within less than or equal to 28 days since the beginning of the treatment with a compound described herein. In some embodiments, the therapeutic effect is a decrease from baseline in HAM-D total score after treatment with a compound described herein (e.g., treatment with a compound described herein once a day for 14 days). In some embodiments, the HAM-D total score of the subject before treatment with a compound described herein is at least 24. In some embodiments, the HAM-D total score of the subject before treatment with a compound described herein is at least 18. In some embodiments, the HAM-D total score of the subject before treatment with a compound described herein is between and including 14 and 18. In some embodiments, the decrease in HAM-D total score after treating the subject with a compound described herein relative to the baseline HAM-D total score is at least 10. In some embodiments, the decrease in HAM-D total score after treating the subject with a compound described herein relative to the baseline HAM-D total score is at least 15 (e.g., at least 17). In some embodiments, the HAM-D total score associated with treating the subject with a compound described herein is no more than a number ranging from 6 to 8. In some embodiments, the HAM-D total score associated with treating the subject with a compound described herein is no more than 7.

In some embodiments, the method provides therapeutic effect (e.g., as measured by reduction in Clinical Global Impression-Improvement Scale (CGI)) within 14, 10, 4, 3, 2, or 1 days, or 24, 20, 16, 12, 10, or 8 hours or less. In some embodiments, the CNS-disorder is a depressive disorder, e.g., major depressive disorder. In some embodiments, the method of treating the depressive disorder, e.g., major depressive disorder provides a therapeutic effect within the second day of the treatment period. In some embodiments, the therapeutic effect is a decrease from baseline in CGI score at the end of a treatment period (e.g., 14 days after administration).

In some embodiments, the method provides therapeutic effect (e.g., as measured by reduction in Montgomery-Asberg Depression Rating Scale (MADRS)) within 14, 10, 4, 3, 2, or 1 days, or 24, 20, 16, 12, 10, or 8 hours or less. In some embodiments, the CNS-disorder is a depressive disorder, e.g., major depressive disorder. In some embodiments, the method of treating the depressive disorder, e.g., major depressive disorder provides a therapeutic effect within the second day of the treatment period. In some embodiments, the therapeutic effect is a decrease from baseline in MADRS score at the end of a treatment period (e.g., 14 days after administration).

A therapeutic effect for major depressive disorder can be determined by a reduction in Montgomery-Asberg Depression Rating Scale (MADRS) score exhibited by the subject. For example, the MADRS score can be reduced within 4, 3, 2, or 1 days; or 96, 84, 72, 60, 48, 24, 20, 16, 12, 10, 8 hours or less. The Montgomery-Asberg Depression Rating Scale (MADRS) is a ten-item diagnostic questionnaire (regarding apparent sadness, reported sadness, inner tension, reduced sleep, reduced appetite, concentration difficulties, lassitude, inability to feel, pessimistic thoughts, and suicidal thoughts) which psychiatrists use to measure the severity of depressive episodes in patients with mood disorders.

In any and all aspects, in some embodiments, the compound for use in a method described herein is selected from:

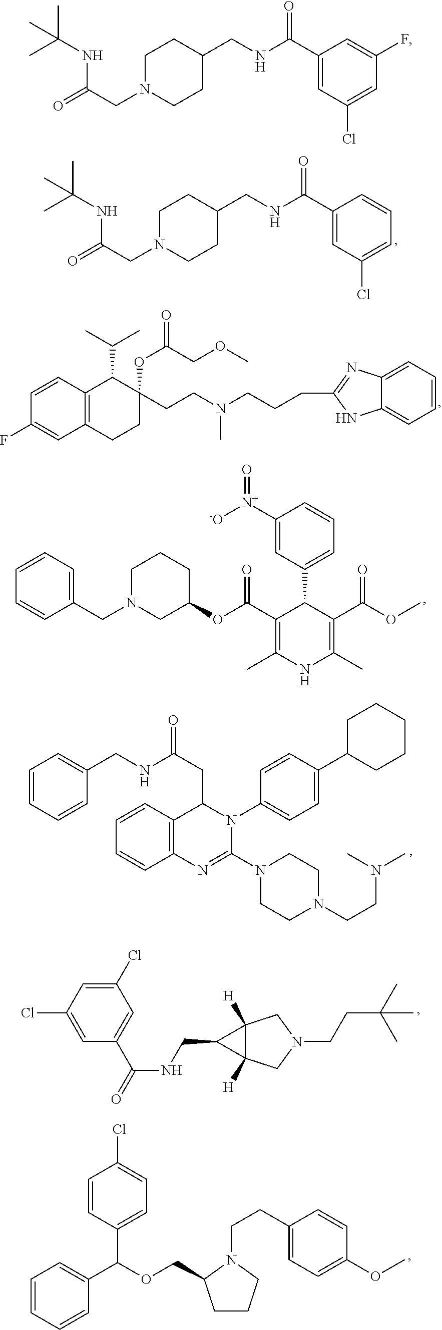

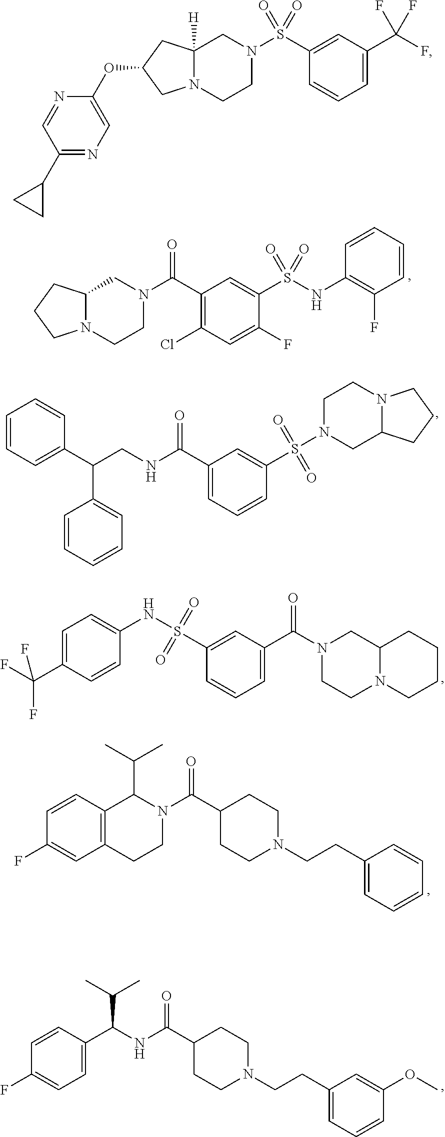 [SMILES: O=S(=O)(c1cccc(C(F)(F)F)c1)N1CCN2C[C@H](Oc3cnc(C4CC4)cn3)C[C@H]2C1 ; O=C(c1cc(S(=O)(=O)Nc2ccccc2F)c(F)cc1Cl)N1CCN2CCC[C@@H]2C1 ; O=C(NCC(c1ccccc1)c1ccccc1)c1cccc(S(=O)(=O)N2CCN3CCCC3C2)c1 ; O=C(c1cccc(S(=O)(=O)Nc2ccc(C(F)(F)F)cc2)c1)N1CCN2CCCCC2C1 ; CC(C)C1c2ccc(F)cc2CCN1C(=O)C1CCN(CCc2ccccc2)CC1 ; COc1cccc(CCN2CCC(C(=O)N[C@@H](c3ccc(F)cc3)C(C)C)CC2)c1]

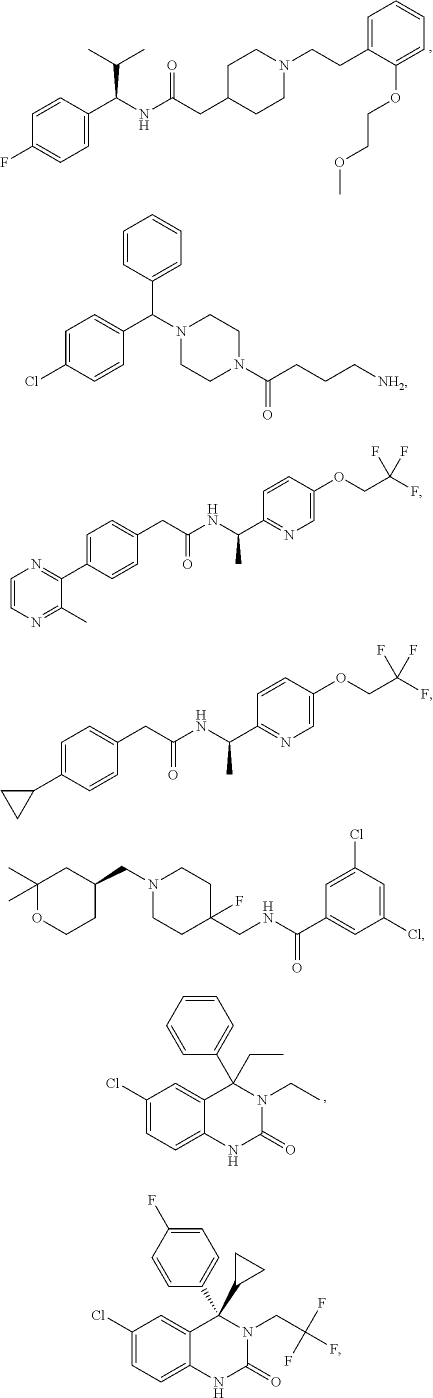

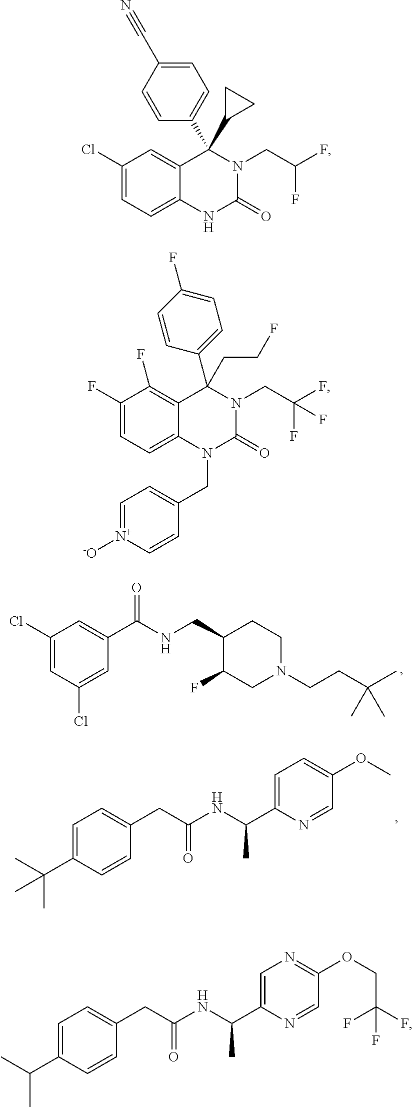

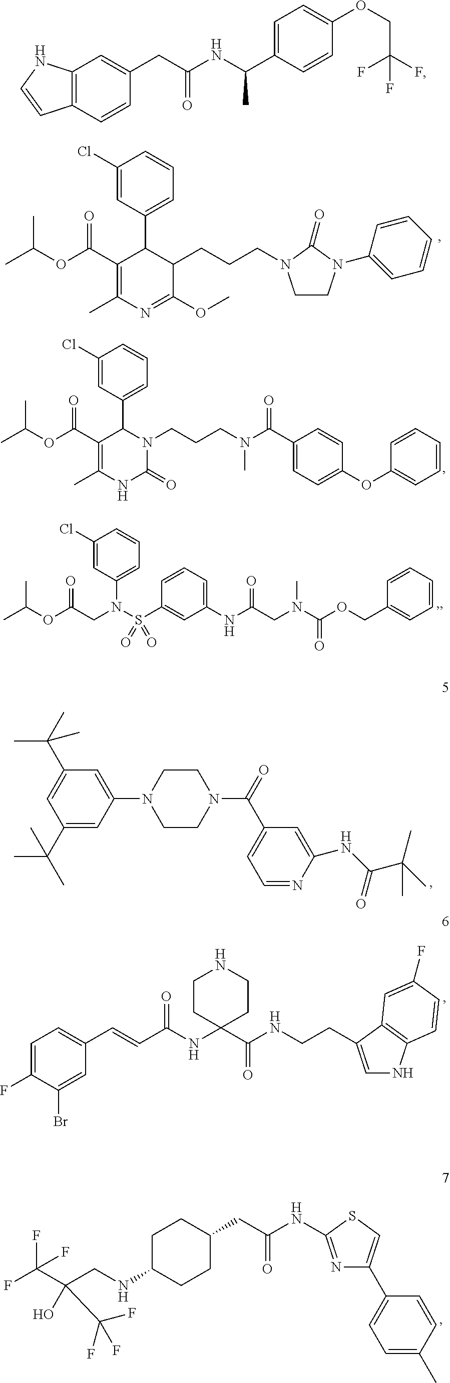

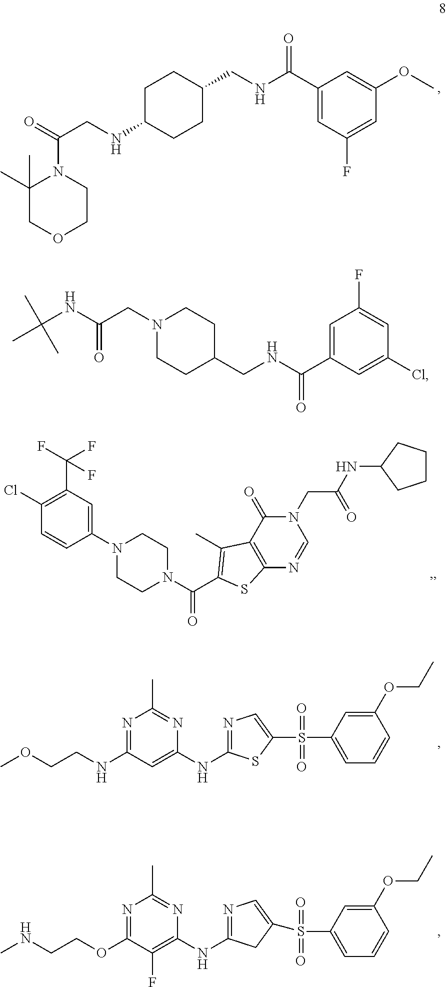

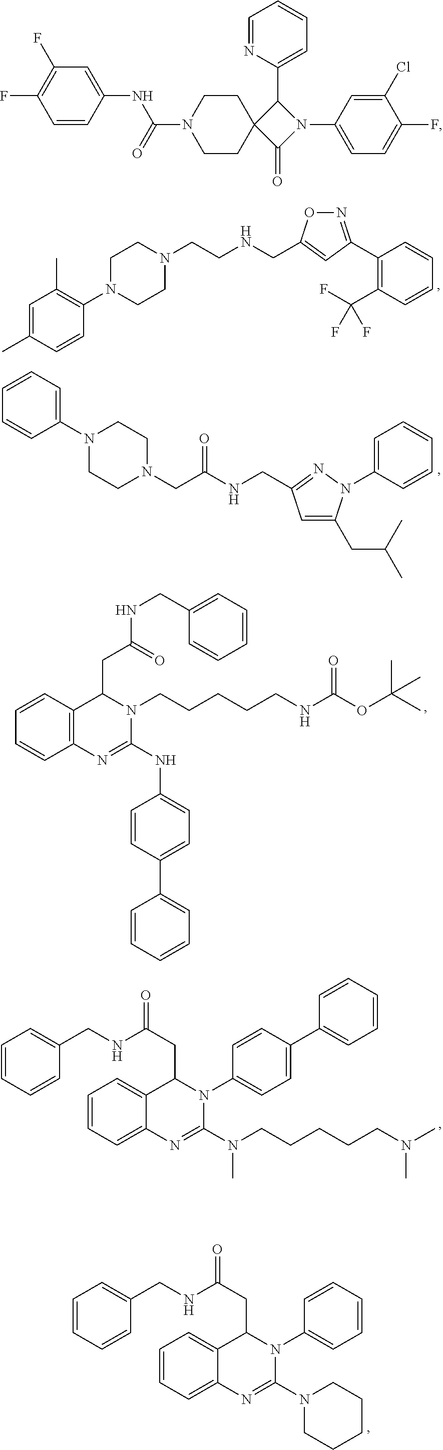

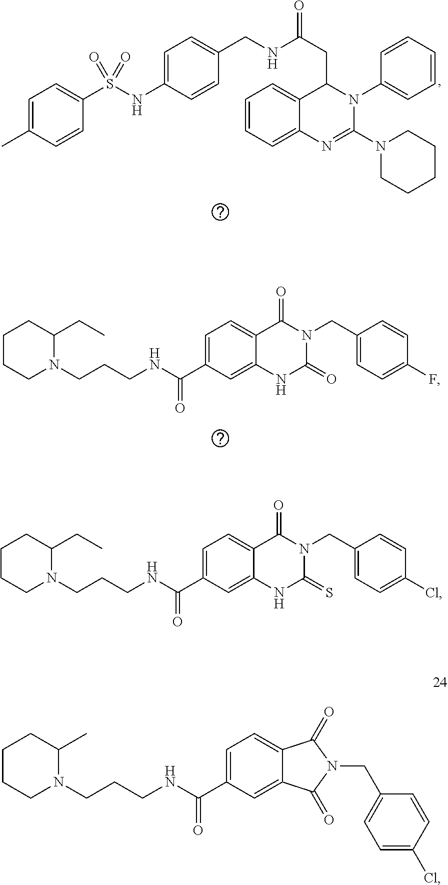 [SMILES: Cc1ccc(S(=O)(=O)Nc2ccc(CNC(=O)CC3c4ccccc4N=C(N4CCCCC4)N3c3ccccc3)cc2)cc1 ; CCC1CCCCN1CCCNC(=O)c1ccc2c(=O)n(Cc3ccc(F)cc3)c(=O)[nH]c2c1 ; CCC1CCCCN1CCCNC(=O)c1ccc2c(=O)n(Cc3ccc(Cl)cc3)c(=S)[nH]c2c1 ; CC1CCCCN1CCCNC(=O)c1ccc2c(c1)C(=O)N(Cc1ccc(Cl)cc1)C2=O]

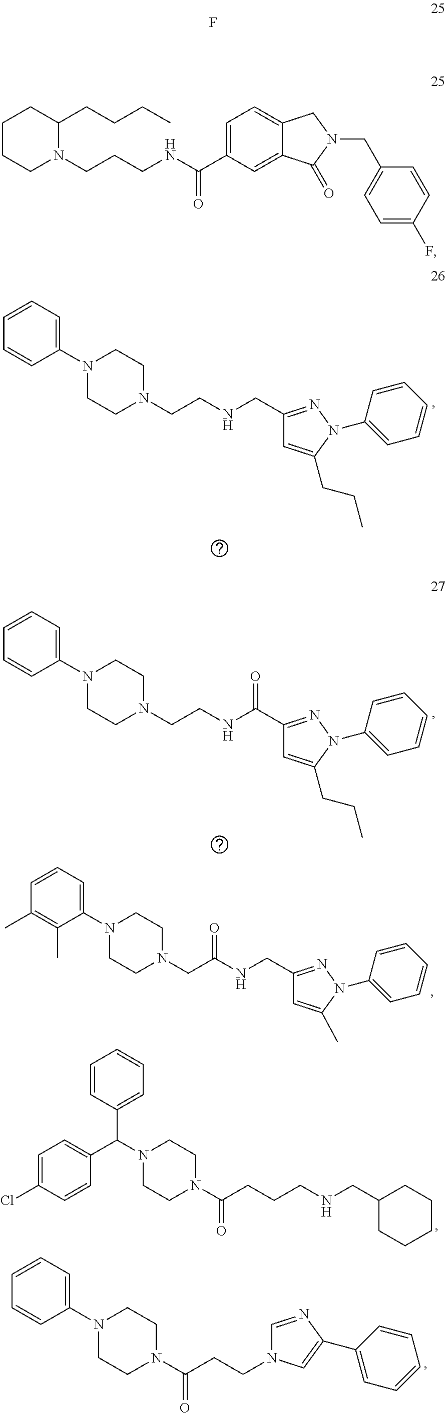

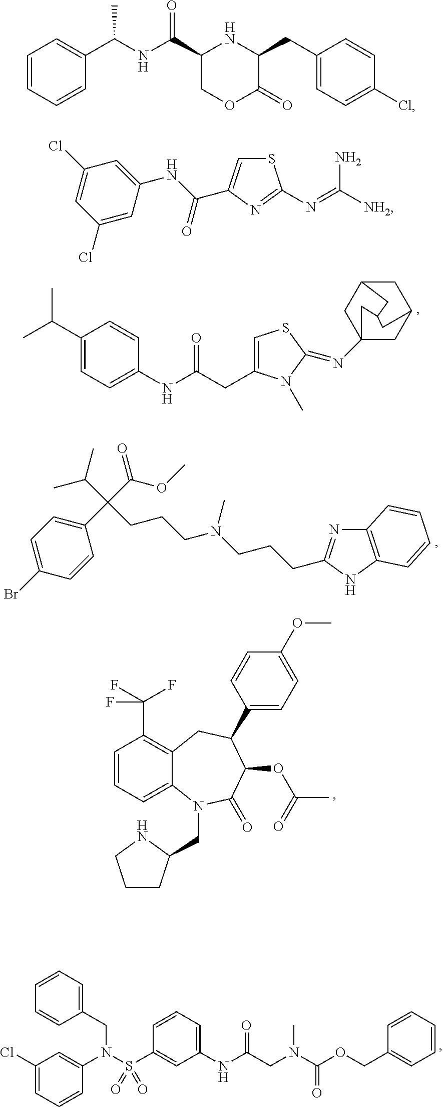

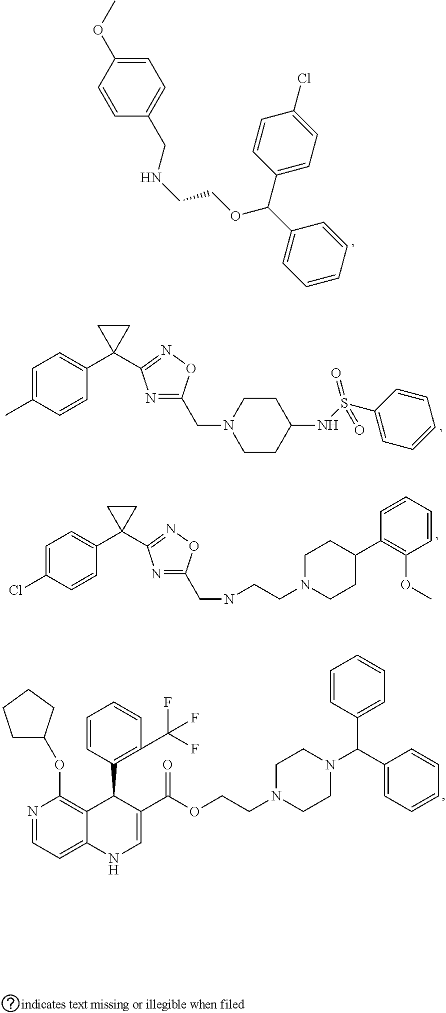

N-[(1R)-1-(4-fluorophenyl)-2-methylpropyl]-1-[2-(3-methoxyphenyl)ethyl]piperidine-4-carboxamide, 6-fluoro-1-isopropyl-2-{[1-(2-phenylethyl)piperidin-4-yl)carbonyl-1,2,3,4-tetrahydroisoquinoline, diltiazem, tylerdipine, P-11520031, DP-3005, RQ-00311610, A-1264087, A-1315647, VMD-3816 or VMD-3222, or a pharmaceutically acceptable salt thereof.

### Pharmaceutical Compositions and Routes of Administration

Compounds provided in accordance with the present invention are usually administered in the form of pharmaceutical compositions. This invention therefore provides pharmaceutical compositions that contain, as the active ingredient, one or more of the compounds described, or a pharmaceutically acceptable salt thereof, and one or more pharmaceutically acceptable excipients, carriers, including inert solid diluents and fillers, diluents, including sterile aqueous solution and various organic solvents, permeation enhancers, solubilizers and adjuvants. The pharmaceutical compositions may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.)

The pharmaceutical compositions may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

In some embodiments, the compounds and compositions described herein are administered orally. The compound or a composition thereof may be formulated in a liquid or oral dosage form. Administration may be via capsule or tablet (e.g., an enteric coated tablet), or the like. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of a tablet, pill, powder, lozenge, sachet, elixir, suspension, emulsion, solution, syrup, aerosol (as a solid or in a liquid medium), or ointment containing, for example, up to 10% by weight of the active compound, or capsule (e.g., soft or hard gelatin capsule).

In some embodiments, the compounds and compositions described herein are administered parenterally, e.g., by injection or intravenously. The compound or a composition thereof may be formulated in a liquid dosage form, and may include one or more excipients.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

The compositions of the invention can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. Preferably, for oral administration, each dosage unit contains from 1 mg to 2 g of a compound described herein, and for parenteral administration, preferably from 0.1 to 700 mg of a compound a compound described herein. It will be understood, however, that the amount of the compound actually administered usually will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

### Combination Therapy

A compound or composition described herein (e.g., for use in modulating a T-type calcium ion channel) may be administered in combination with another agent or therapy. A subject to be administered a compound disclosed herein may have a disease, disorder, or condition, or a symptom thereof, that would benefit from treatment with another agent or therapy. These diseases or conditions can relate to epilepsy or an epilepsy syndrome (e.g., absence seizures, juvenile myoclonic epilepsy, or a genetic epilepsy).

### Antiepilepsy Agents

Anti-epilepsy agents include brivaracetam, carbamazepine, clobazam, clonazepam, diazepam, divalproex, eslicarbazepine, ethosuximide, ezogabine, felbamate, gabapentin, lacosamide, lamotrigine, levetiracetam, lorazepam, oxcarbezepine, permpanel, phenobarbital, phenytoin, pregabalin, primidone, rufinamide, tigabine, topiramate, valproic acid, vigabatrin, zonisamide.

### Analgesics

Analgesics are therapeutic agents that are used to relieve pain. Examples of analgesics include opiates and morphinomimetics, such as fentanyl and morphine; paracetamol NSAIDs, and COX-2 inhibitors. Given the ability of the compounds of the invention to treat pain via inhibition of T-type calcium channels (e.g., Cav3.1, Cav3.2, and Cav3.3), combination with analgesics are particularly envisioned.

### EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

### Example 1: Testing of Z941 and Z944 in a Model of Genetic Generalized Epilepsy with Persistent Absence Seizures

Absence seizures are a common seizure type in children with genetic generalized epilepsies. Absence seizures are generalized, non-convulsive seizures characterized by temporary behavioural immobility and unresponsiveness that is accompanied by a distinct pattern of bilateral spike-and-wave discharges (SWDs) on an electroencephalogram (EEG) recording. The absence seizures are associated with hypersynchronous oscillatory burst firing in both the thalamus and the cortex. Although the molecular mechanisms underlying the development of epilepsy are poorly understood, T-type calcium (Ca²⁺) channels are critically involved because they are essential for the initiation and propagation of neuronal firing and hence for seizure generation and maintenance. Absence seizures most commonly affect children and adolescents and can persist during adulthood, becoming very difficult to treat; these individuals can experience hundreds of seizures per day, which can disrupt learning and affect behaviour and motor skills. Ethosuximide (ETX), a first-line drug used to treat patients with absence epilepsy, is widely believed to act by the pan-blockade of T-type Ca²⁺ channels in the millimolar plasma concentration range. However, ETX also affects other ionic conductances. Nevertheless, a recent blinded randomized comparative trial demonstrated that more than 40% of patients continue to have absence seizures despite treatment with ETX.

GAERS are a well-validated genetic rat model of GGE with absence seizures that show generalized SWDs that have an abrupt onset and offset on a normal EEG background, closely resembling those seen during human absence seizures. During the seizures, which usually last from 5 to 30 s, behaviourally, the rats show arrest of activity and repetitive head nodding. The therapeutic profile of the seizures in GAERS is similar to that of human absences, being inhibited or exacerbated by similar antiepileptic drugs.

### Methods Systemic Administration of Z944

In vivo anti-seizure activity of Z941 and Z944 was assessed in eight female epileptic GAERS (180 to 250 g and 18 to 26 weeks). Rats were implanted with extradural recording electrodes. EEG data were acquired at a sampling rate of 256 Hz unfiltered using Compumedics ProFusion digital EEG acquisition software. After 60-min habituation, rats received intraperitoneal injections of drug, and after a further 15 min, the EEG was acquired for 120 min—the test period. Drug treatments were randomized, with at least 48 hours between treatments, and consisted of Z944 (10 or 30 mg/kg), Z941 (10 or 30 mg/kg), ETX (100 mg/kg in 0.9% saline; Sigma), sodium valproate (200 mg/kg in 0.9% saline; Sigma), or the vehicle for Z941 and Z944 (10% DMSO in 0.5% carboxymethylcellulose). Over a 5-week period, each rat received all treatments (crossover design) in a randomized manner with a 48-hour washout period between dosing of test compound, control vehicle, and positive control articles. Each drug was coded such that the experimenter was blinded to the drug being administered. Clinical observations of neurotoxic adverse effects were assessed every 15 min throughout the 120-min test period. These were quantified according to an ordinal scale of 0 to 4: 0, no sedation, normal movement; 1, slight sedation, slow movement but alert when startled; 2, mildly sedate, reduced struggle to restraint; 3, sedate, not moving in cage, but does respond to provocation; 4, very sedate, catatonic and unable to stand when provoked. The eight scores (taken for one experiment per rat) were then averaged, and group means were calculated for each treatment. Animals were also monitored daily for general health throughout the study period (that is, weight gain and fur condition).

SWDs were detected automatically with the Mighty EDF1 EEG viewing software (version 1.3.3), custom-designed to quantify seizures in GAERS, and subsequently manually checked by an investigator blinded to the treatment group of the animals. The following criteria were used: SWD burst of amplitude of more than three times baseline, a frequency of 6 to 12 Hz, and duration of longer than 0.5 s. The total percentage time spent in seizure activity, the average seizure duration, and the number of seizures were calculated for each experiment. The cycle frequency of the SWDs (Hz) was analyzed for the highest dose of the Z941 (30 mg/kg) and Z944 (30 mg/kg) compared to the vehicle DMSO and ethosuximide treatments. The analysis was performed with Clampfit 10.2 software (Molecular Devices). For each rat for each of the four treatments, the frequency of SWD was measured by obtaining the average cycle frequency of the first 10 seizures during the 120-min target period. Note that for some traces, there were no seizures or fewer than 10 seizures during the entire 120-min period.

### Evaluation of Delta Wave Activity of Z944 in GAERS

Interictal EEG traces from GAERS receiving Z944 (10 mg/kg), ethosuximide (100 mg/kg), or vehicle intraperitoneally were analyzed for delta wave power with NeuroScan software (Compumedics). For this, the EEG recording for the first 45 min after the drug administration was selected. The 45-min block was then broken into 1-min intervals, which were analyzed with 2-s epochs. The epochs were manually reviewed, and any containing seizure activity or contaminated by artifact was excluded from the analysis. An FFT was applied to the remaining epochs, and the power for the delta activity (0 to 3.75 Hz) in each window was calculated. Any outliers, which were determined as points in which an individual value was greater than or less than twice the SD for the relevant band power, were removed from analysis.

### Thalamic Slice Patch-Clamp Recordings

P10-P20 GAERS and non-epileptic control (NEC) rats were briefly anesthetized with halothane and killed by cervical dislocation, and the brains were rapidly removed. Brain tissue was glued to a cutting chamber, which was filled with ice-cold sucrose solution containing 234 mM sucrose, 24 mM NaHCO3, 1.25 mMNaH2PO4, 11 mM glucose, 2.5 mM KCl, 0.5 mM CaCl2, and 10 mMMgSO4, bubbled with 95% O2/5% CO2. Horizontal brain slices (350-mm thick) were cut from the level of the ventral nRT and incubated for a minimum of 1 hour at 34° C. in recording solution containing 126 mM NaCl, 2.5 mM KCl, 26 mM NaHCO3, 1.25 mM NaH2PO4, 2 mM CaCl2), 2 mM MgCl2, 10 mM glucose, 1 mM kynurenic acid, and 0.1 mM picrotoxin, bubbled with 95% O₂/5% CO₂.

Slices were then transferred to the recording chamber superfused with recording solution and maintained at 33° C. to 35° C. nRT neurons were visualized with a DIC microscope (Axioskop 2-FS Plus, Carl Zeiss) and infrared camera (IR-1000, Dage-MTI) and visually identified by their morphology and orientation. All recordings were undertaken with a Multiclamp 700B amplifier and pCLAMP software version 9 (Molecular Devices). The recording chamber was grounded with an Ag/AgCl pellet. Whole-cell voltage-clamp recordings were undertaken with fire-polished borosilicate glass pipettes (3 to 5 megohms) filled with an intracellular of composition containing 140 mM Cs-methanesulfonate, 10 mM Hepes, 0.5 mM MgCl2, 11 mM EGTA, 1 mM CaCl2, 5 mM tetraethylammonium-Cl, 4 mM MgATP, and 0.5 mM NaGTP (sodium guanosine triphosphate) (pH adjusted to 7.2 with CsOH and osmolarity adjusted to 290 mOsm/kg with D-mannitol). TTX (600 nM), 4-aminopyridine (2 mM), tetraethylammonium-Cl (10 mM), CdCl2 (50 mM), and nimodipine (1 mM) were added to the recording solution to reduce contamination from non-LVA T-type Ca2+ channel currents. The liquid junction potential for voltage-clamp solutions was calculated as +9.7 mV and corrected online.

To construct a concentration dependent response curve, we superfused the cells with Z944 after stable baseline recording. Percentage block of T-type Ca2+ channel current was calculated, and pooled data were plotted on a log scale and fitted with a Hill equation (Eq. 5), where y=fraction of binding sites filled, Kd=dissociation constant, L=ligand concentration, and n=Hill coefficient.

Current density for the HVA and LVA current was measured by applying 200-ms depolarizing test steps at 5-mV increments from −85 to 0 mV, from a holding potential of −90 mV. This protocol was repeated with a 50-ms depolarizing step to −20 mV preceding the test step, which removed the fast-inactivating LVA component, isolating the HVA component. The isolated HVA component was then subtracted from the HVA+LVA current recorded previously to isolate only the T-type Ca2+ current. This was then normalized to whole-cell capacitance to yield the current density. Currents recorded under voltageclamp conditions were sampled at 20 kHz and filtered at 2.4 kHz, and leak current was subtracted with online P/5 subtraction. Whole-cell current-clamp recordings were undertaken with firepolished borosilicate glass pipettes (4 to 6 megohms) filled with the following solution containing 120 mM K-gluconate, 10 mM Hepes, 1 mM MgCl2, 1 mM CaCl2, 11 mM KCl, 11 mM EGTA, 4 mMMgATP, and 0.5 mM NaGTP (pH adjusted to 7.2 with KOH and osmolarity adjusted to 290 mOsm/kg with D-mannitol). The liquid junction potential for current-clamp solutions was calculatedas+13.3 mV and corrected off-line. To evaluate cell response to hyperpolarization and depolarization, we injected the current from −110 to +190 pA in 10-pA increments for a duration of 1.2 s at the cell's intrinsic RMP.Neurons that did not exhibit burst firing (as determined by a minimum of three action potentials within 150 ms of the current step) in response to depolarizing current steps were discarded. Voltage responses under current-clamp conditions were sampled at 50 kHz and filtered at 10 kHz. Data analysis was performed with Clampfit 9 software and Origin version 7.5. Data followed a normal distribution, and statistical significance was calculated with one-way ANOVA with Tukey's post hoc test considering a P value of <0.05 as significant. Data were plotted as means±SEM.

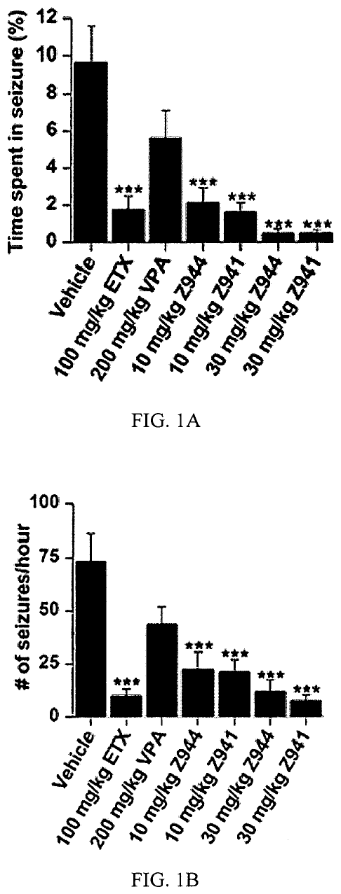

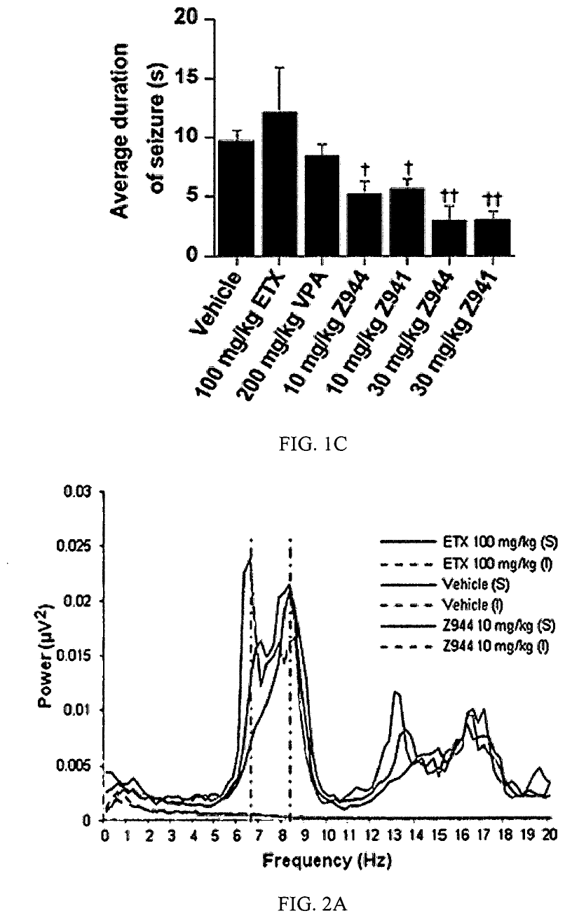

### FIGS. 1A, 1B, 1C. Effect of Drugs on Seizures in GAERS.

Data represent (FIG. 1A) the effect of drug treatment on the percentage of time spent in seizure activity, (FIG. 1B) the average number of seizures per hour, and (FIG. 1C) the average seizure duration during the 120-min post-drug test period. Eight animals were used with a repeated-measures design. ***p<0.001 compared to vehicle; †p<0.05 or ††P<0.01 compared to ethosuximide (ETX). VPA, valproate.

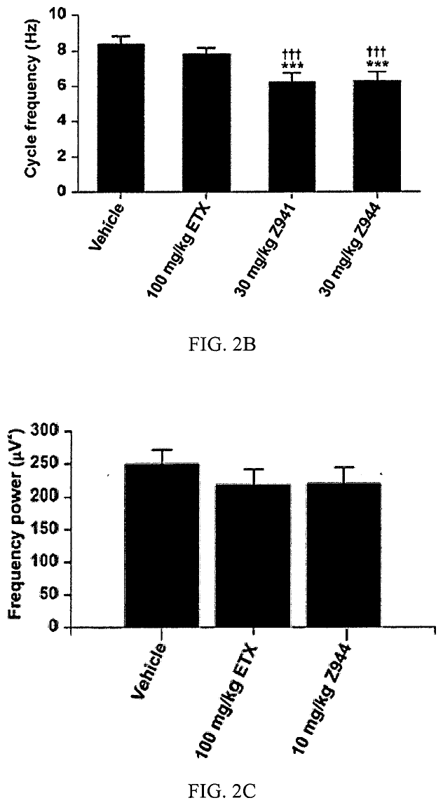

### FIGS. 2A, 2B, 2C. Effect of Drugs on Cycle Frequency of SWDs.

(FIG. 2A) FFT of Z944, ethosuximide (ETX), and vehicle during both seizure (S) activity and interictal (I) activity. The graph shows a shift in peak cycle frequency power from 8 to 6.5 Hz during seizure activity in Z944 (30 mg/kg)-treated animals when compared to ethosuximide (100 mg/kg) and vehicle. FFTs were averaged over five interictal or seizure periods for each treatment. (FIG. 2B) Mean cycle frequency of the SWDs (cycles per second) for the highest dose of the two Ca2+ channel blockers Z941 (30 mg/kg) and Z944 (30 mg/kg) compared to ethosuximide (100 mg/kg) and the vehicle. The cycle frequency of SWDs was obtained by measuring the average cycle frequency of the first 10 clean seizures during the 120-min target period. (FIG. 2C) Quantification of the power of delta frequency activity (0 to 3.75 Hz.) on the EEG recordings during interictal periods in GAERS acquired for a 45-min period after intraperitoneal administration of Z944 (10 mg/kg), ethosuximide (100 mg/kg), or vehicle while the animals were awake and freely moving. The frequency power is plotted as mean SEM, n=8 animals. ***P<0.001 compared to vehicle; †††P<0.001 compared to ethosuximide.

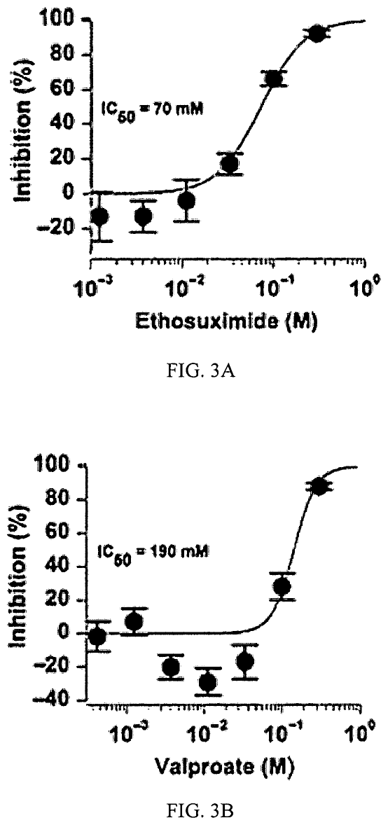

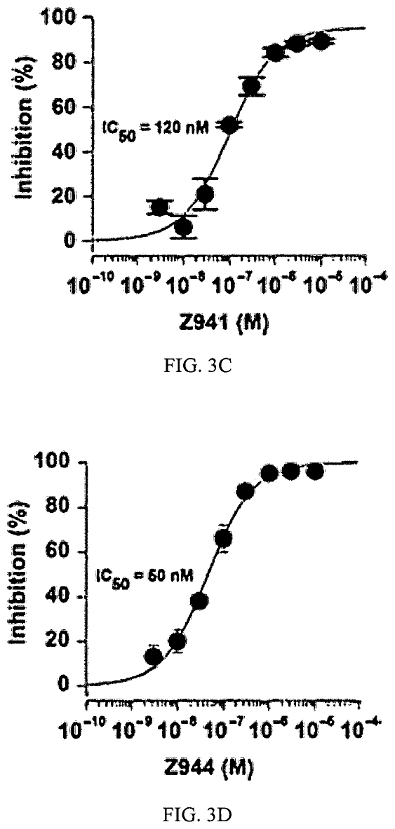

FIGS. 3A, 3B, 3C, 3D. Concentration-Dependent Inhibition of hCa_{V}3.2 Measured with an Inactivated Channel State FLIPR Assay.

(FIG. 3A) Ethosuximide IC50 inactivated state=70 mM, slope=2.1. (FIG. 3B) Valproate IC50 inactivated state=190 mM, slope=4.4. (FIG. 3C) Z941 IC50 inactivated state=120 nM, slope=0.8. (FIG. 3D) Z944 IC50 inactivated state=50 nM, slope=1.5. Data are means±SD. n=4 wells per concentration fitted with a logistic function.

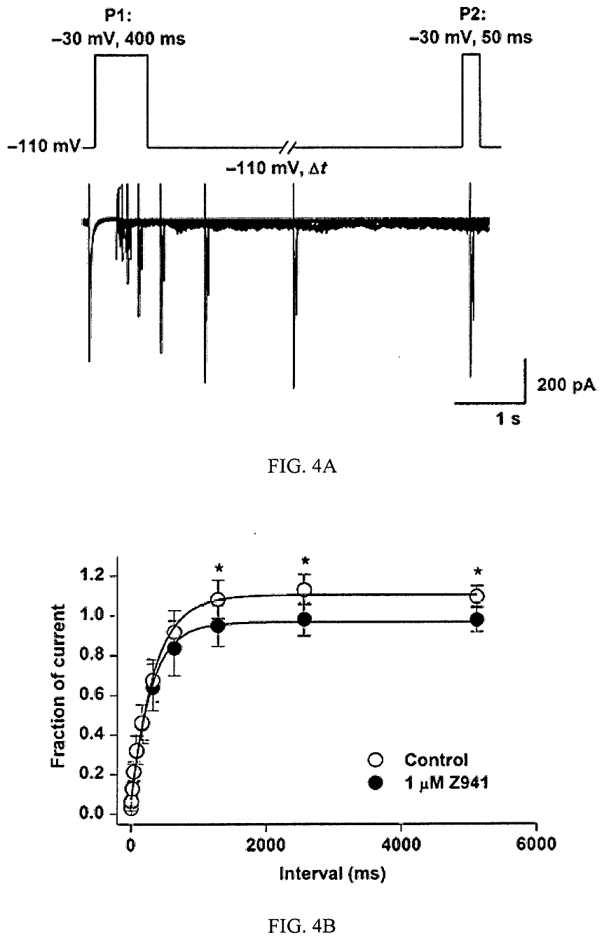

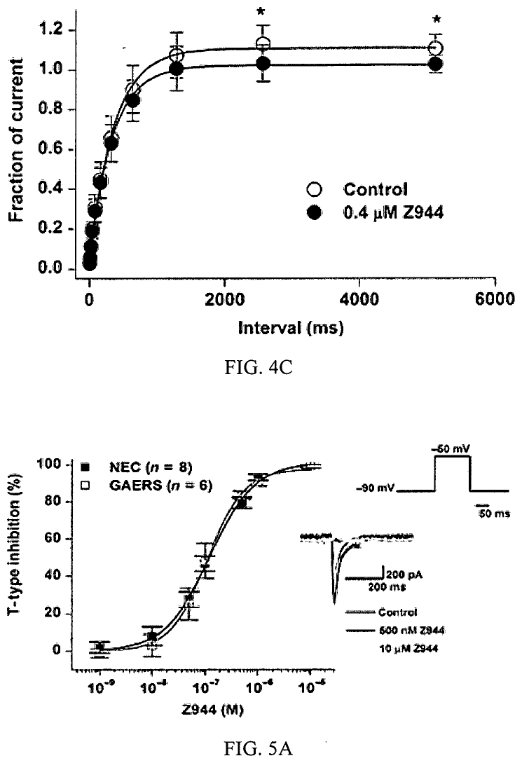

FIGS. 4A, 4B, 4C. Effect of Z941 and Z944 on rCaV3.2.

(FIG. 4A) The kinetics of recovery from inactivation were determined with a two-pulse protocol (P1, 400 ms to −30 mV; P2, 50 ms to −30 mV) with a variable interpulse interval (5 to 5120 ms) applied every 20 s from a holding potential of −110 mV. Below are representative CaV3.2 channel current traces. (FIGS. B and C) The time course of recovery from channel inactivation was fitted with a single exponent for currents recorded in the presence or absence of Z941 and Z944. t=354±80 ms recorded in 0.02% DMSO, 312±71 ms in 1 mM Z941 (B), and 354±80 ms in 0.4 mM Z944. (C) Data are means±SD. n=5 to 11 cells per concentration. *P<0.05, Student's t test, compared to 0.02% DMSO control.

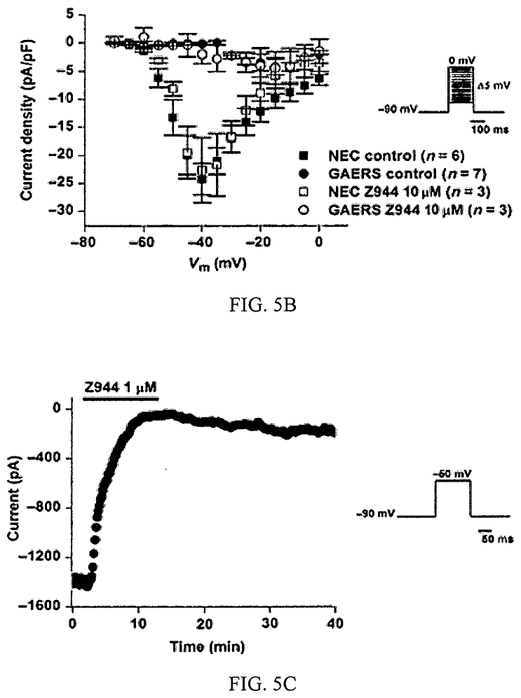

### FIGS. 5A, 5B, 5C. Effect of Z944 on Thalamic nRT T-Type Ca2+ Currents.

(FIG. 5A) Concentration dependent response curve generated by inhibition of the mixed CaV3.2/CaV3.3 current expressed in NEC (filled squares; n=8) and GAERS (empty squares; n=6) nRT neurons by Z944. Inset: Representative traces of a GAERS nRT T-type Ca2+ channel currents in the absence (black trace) or presence of 500 nM Z944 (dark gray trace) and 10 mM Z944 (light gray trace). Currents were elicited by a step to −50 mV from a holding of −90 mV for a duration of 150 ms every 10 s until a stable baseline was achieved, then various concentrations of Z944 were applied. (FIG. 5B) Current density plot demonstrating GAERS nRT currents in the absence (filled circles; n=7) and presence (open circles; n=3) of 10 mM Z944. Inset: Currents were elicited at various potentials beginning at −70 mV (duration, 200 ms) and then in increasing increments of 5 mV from a holding potential of −90 mV every 10 s. (FIG. 5C) Representative time course demonstrating nonreversible inhibition of GAERS nRT T-type Ca2+ channel currents under voltage clamp in response to 1 mM Z944 and wash off. Inset: Currents were elicited by a step to −50 mV from a holding of −90 mV for a duration of 150 ms every 10 s. Data are means±SEM.

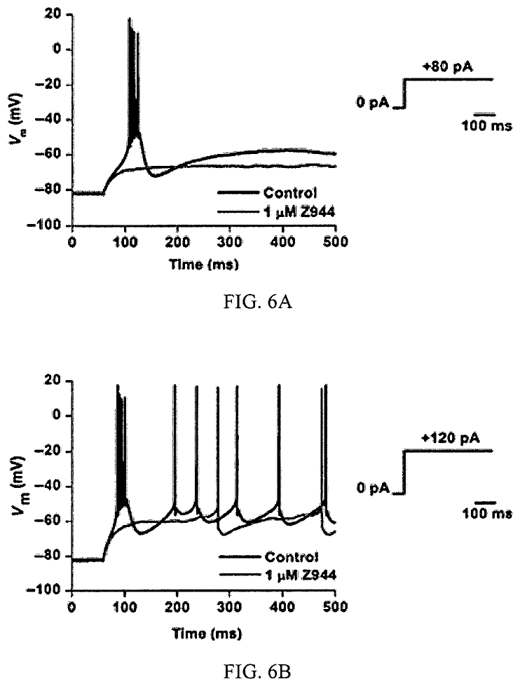

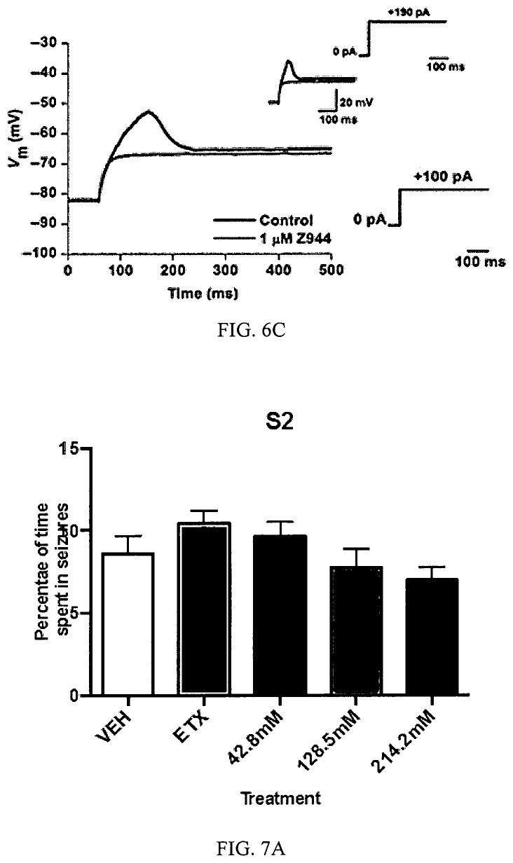

### FIGS. 6A, 6B, 6C. Effect of Z944 on Burst Firing in Thalamic nRT Neurons.

(FIG. 6A) Representative traces from a GAERS nRT neuron in current clamp, showing voltage responses to a depolarizing current (+80 pA; inset), which is the threshold for burst firing in control (black trace) but the subthreshold for firing after application of 1 mM Z944 (gray trace). (FIG. 6B)Representative traces from the same GAERS nRT neuron as in (A), showing voltage responses to a depolarizing current of greater magnitude (+120 pA; inset), which is suprathreshold for burst firing in control (black trace) but threshold for tonic firing after application of 1 mMZ944 (gray trace). (FIG. 6C) Representative traces from a GAERS nRT neuron in the presence of 600 nM TTX, showing voltage responses to a depolarizing current (+100 pA; lower inset), which is threshold for low threshold spiking in control (black trace) but subthreshold for low-threshold spiking after application of 1 mM Z944 (gray trace). Upper inset: Representative traces from the same GAERS nRT neuron, showing voltage responses to a depolarizing current of greater magnitude (+190 pA; inset), which is suprathreshold for low-threshold spiking in control (black trace) and still subthreshold after application of 1 mM Z944 (gray trace).

### Example 2: Focal Injection Studies

24 week old GAERS (n=9) were used for the secondary somatosensory cortex (S2) focal injection study GAERS (n=8) were used for the thalamic reticular nucleus (nRT) focal injection study.

Rats were implanted with two intra-cerebral guide cannulae with injection needles in the S2 AP±0.2 mm, ML±5.8 mm, DV±2.5 mm or the nRT AP±1.8 mm, ML±2.9 mm, DV±5.6 mm. Each animal received two sham saline treatments prior to beginning the actual experiment to become accustomed to the procedure. Each animal received each of the five treatments (42.8 mM, 128.5 mM and 214.2 mM of Z944, 800 mM ETX, vehicle) once every fourth day and therefore acted as its own control. Compumedics ProFusion EEG software was used to take EEG recordings During the 30 min baseline recording period each animal had to have a minimum of 14 seizures or it was excluded from the study that day. Treatment was delivered over a period of 10 minutes for the S2 cohort and over a period of 2 minutes for the nRT cohort. After a 15 minute post-drug habituation period the EEG were recorded for 90 minutes.

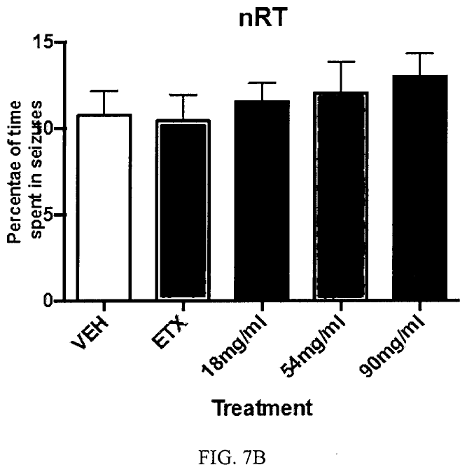

### FIGS. 7A, 7B. Effects of Focal Injection of Z944 in GAERS.

The graphs show the percentage of time the animals spent in seizure activity during the 90-minuto EEG recordings after treatment. In (FIG. 7A) Somatosensory cortex S2 and (FIG. B) thalamic reticular nucleus (nRT) the different treatments did not significantly reduce the number of seizures in the animals.

### Example 3: Antiepileptogenic Effects of Z944 in Rat Models of Temporal Lobe Epilepsy

Temporal lobe epilepsy (TLE) is the most common form of acquired epilepsy in adults, and is often drug resistant. Current therapeutic treatment is symptomatic, suppressing seizures, but has no disease modifying effect on epileptogenesis. Epileptogenesis is a series of processes that lead to the development of spontaneous recurring seizures and is a sequel from a brain insult, such as status epilepticus (SE), traumatic brain injury or stroke. While the mechanisms that are fundamentally critical to epileptogenesis are not completely understood several studies have associated changes in T-type Ca²⁺ channels and intracellular Ca²⁺ levels with the development of TLE. Therefore, we examined the antiepileptogenic effects of Z944 in 2 models of TLE.

### The Amygdala Kindling Model Experiments

The kindling model has been used extensively as a functional model of TLE. In kindling, repetitive electrical stimulation results in a progressive increase in the severity and duration of the seizures. Once the rat has been kindled, the intensified response to the electrical stimulus seems to be permanent, indicating the development of chronic brain alterations.

Animals were implanted with EEG recording electrodes and with a stimulating bipolar electrode in the left basolateral amygdaloid nucleus using the coordinates AP±3.0 mm, ML±5.9 mm, DV±6.0 mm. The after discharge threshold (ADT) for each animal was determined by administering a stimulation (frequency=60.1 Hz; duration=1000 μs) at one minute intervals until a seizure was clicited. The amplitude of the stimulation was started at 0.02 mA with increments of 0.02 mA; the stimulations were stopped when the animal experienced a seizure of ≥6 second duration, or if the amplitude of 0.40 mA was reached without the occurrence of a seizure. The amplitude at which an animal exhibited a seizure was recorded and used for kindling stimulations throughout the experiment. If the animal did not exhibit a seizure by 0.40 mA, the stimulation was repeated 24 hours later up to a maximum of two repeats. Kindling was performed twice a day for five days per week with an endpoint of 30 stimulations or until the animal presented five class V seizures (referred to as fully kindled). The seizure severity was classified from 0 (less severe) to V (most severe) according to the Racine Scale (Racine, 1972b). Sham kindled animals were handled the same way as the kindled animals, although no electrical stimulation was applied. An animal is classified as fully kindled once it has experienced 5 class V seizures.

### Drug Preparation

Z944 was dissolved in 10% DMSO and 90% Na carboxy-methylcellulose, ethosuximide (ETX) was dissolved in saline and carbamazepine (CBZ) was dissolved in 10% DMSO, 40% propylene glycol and 50% saline.

### Testing Z944 as an Anti-Seizure Agent

Fully kindled rats (i.e. had experienced at least 5 Class V seizures and had a stable seizure response evoked by electrical stimulation) were used for this study (n=7). Drug testing was performed twice a week until all animals had received each of the seven treatments, Z944 (5 mg/kg, 10 mg/kg, 30 mg/kg and 100 mg/kg), vehicle (10% DMSO/90% Na⁺-CMC or CBZ vehicle 20% DMSO/40% propylene glycol/40% saline), ETX (100 mg/kg) or CBZ (30 mg/kg). Prior to drug testing each day, animals were subjected to a maximum of three pre-drug stimulations 30 minutes apart. The animals qualified for testing on that particular day if a class IV or V seizure was elicited in at least 2 out of 3 pre-drug stimulations. Stimulations consisted of a 1 second train of 1 ms biphasic square wave pulses at a frequency of 60 Hz with current amplitude based on each animal ADT that elicited a seizure. Thirty minutes after the last pre-drug stimulation, the animals received an IP injection of one of the seven treatments in a randomized manner at a dose volume of 5 ml/kg. Six post-drug stimulations were administered to each animal, the first stimulation was given 15 minutes post drug injection (to allow brain penetration of the drugs) and the following five stimulations were delivered in 30 minutes intervals thereafter with the seizure class elicited being recorded after each post-drug stimulation. The duration of the primary and total seizures, if any, were recorded. An investigator blinded to the drug treatment analyzed all EEG recordings. Primary after discharge is the duration of the seizure elicited from the kindling stimulation. Total after discharge is the total seizure duration including any secondary seizures that occur after the primary after discharge. Primary after discharge duration and total seizure duration were recorded and averaged for the 6 post-drug stimulations.

Adverse effects of each treatment were recorded, based on a scale of 0-4; where a score of 0 indicates no sedation, normal movement, a score of 1 is for slight sedation, slow movement but alert when startled, a score of 2 is for mildly sedated, struggles when restrained, a score of 3 shows a sedated animal that is not moving in cage, but does respond to provocation and, the highest score of 4 indicates an animal that is very sedate, catatonic and unable to stand when provoked, every 15 minutes from the first post-drug stimulation until 90 minutes and then every half hour thereafter until 150 minutes after the first post-drug stimulation. Animal weight and behaviour when handled were monitored for the duration of the experimental period. Additionally, animals were assessed by observation for any adverse effects of drugs on grooming, fur appearance, gait, and excretion.

Testing Z944 in the progression of seizures in the amygdala kindling model Wistar rats underwent surgery to implant bipolar stimulation electrodes and EEG recording electrodes as described above. ADT was determined as described for the anti-seizure study. Animals were randomly assigned to four different cohorts; Cohort 1: kindled sham+vehicle (n=8); Cohort 2: kindled+vehicle (n=6); Cohort 3: kindled+ETX (100 mg/kg, n=6) and Cohort 4: kindled+Z944 (30 mg/kg, n=7). Thirty minutes prior each kindling stimulation, the animals received an IP injection of vehicle, ETX, or Z944 administered at a dose volume of 5 ml/kg up to an endpoint of 30 stimulations and the seizure class elicited and seizure duration was recorded. None of the animals from the kindled sham+vehicle groups displayed any seizures and were therefore not included in seizure analysis. Adverse effects were recorded every 15 minutes from the drug administration until 90 minutes after. An investigator blinded to the drug treatment analyzed all EEG recordings. The seizure class each stimulation elicited was recorded and averaged and the duration of the primary and total seizures were recorded.

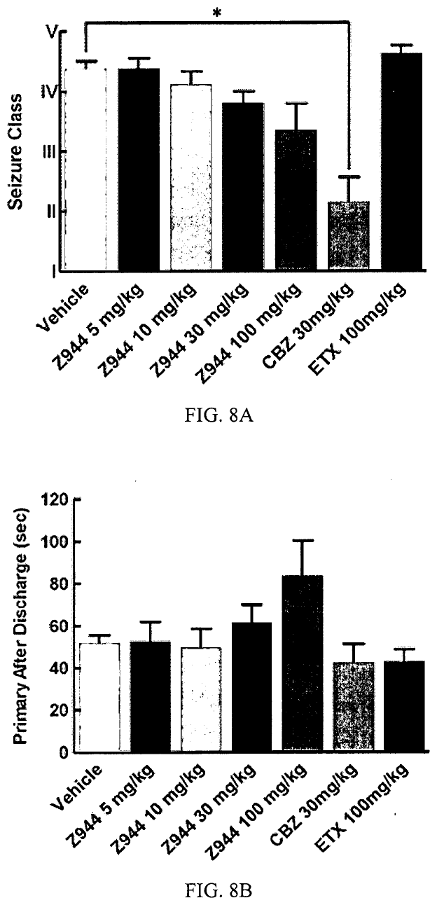

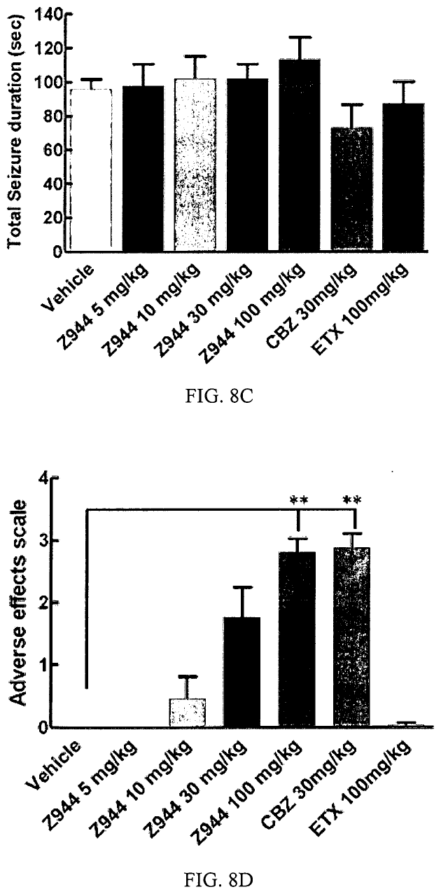

### FIGS. 8A, 8B, 8C, 8D. Z944 has No Anti-Seizure Effect in Fully Kindled Animals.

(FIG. 8A) Z944 does not suppress seizures in fully kindled animals compared to vehicle treatment. Carbamazepine (CBZ, n=7) significantly reduced the class of seizure elicited when compared to vehicle (p<0.05). (FIG. 8B) All four doses of Z944, ETX and CBZ did not affect the primary after discharge when compared to vehicle (p>0.5). (FIG. 8C) Total seizure duration was unaffected in all treatment groups. (FIG. 8D) Adverse effects of Z944 are dose dependent. Z944 (100 mg/kg) and CBZ showed significantly higher adverse effects when compared to vehicle, (p<0.01 for both treatments). *p<0.05, **p<0.01. Kruskal-Wallis test with Dunns post-hoc test.

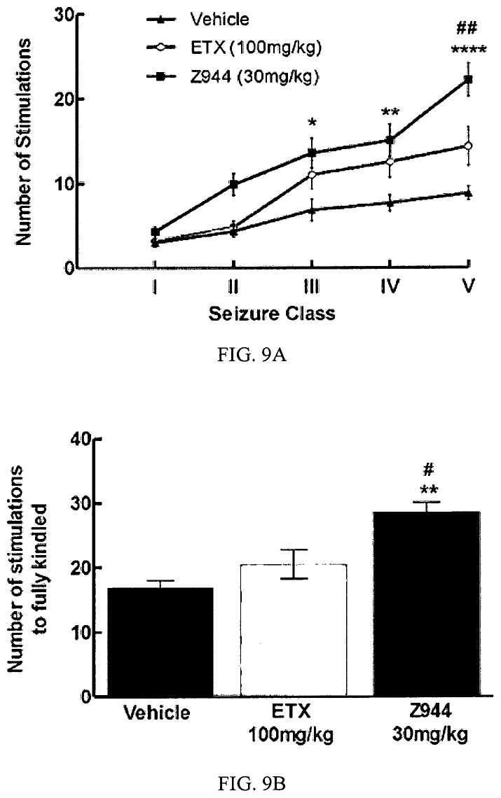

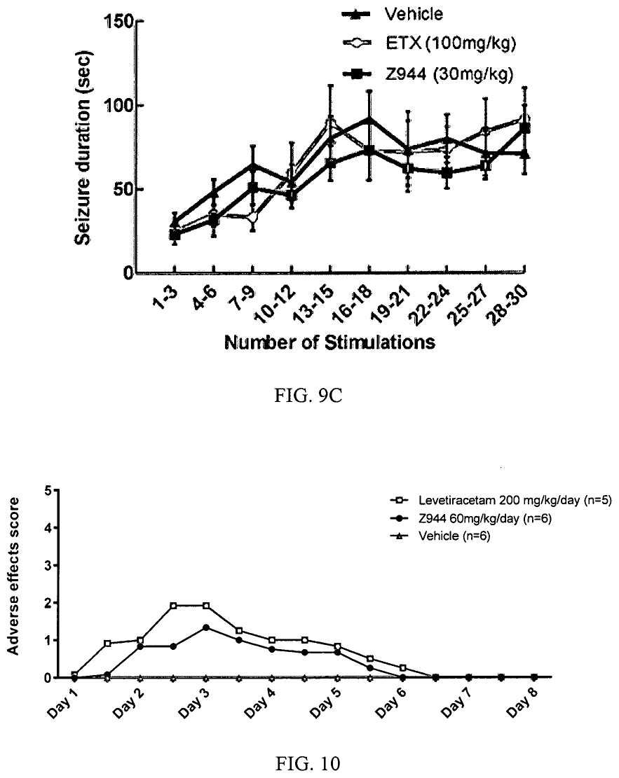

### FIGS. 9A, 9B, 9C. Z944 Delays the Progression to Kindling.

(FIG. 9A) Animals receiving Z944 (n=7, 30 mg/kg) required significantly more stimulations to evoke a class III (p<0.05), IV (p<0.0l) or V (p<0.0001) seizure than the animals receiving vehicle (n=6) and required more stimulations to reach class V (p<0.01) when compared to ethosuximide (ETX, n=6, 100 mg/kg) treated animals. (FIG. 9B) Z944 treated animals (n=7, 30 mg/kg) required significantly more stimulations to reach the fully kindled state when compared to vehicle (n=6, p<0.01) and ETX (n=6, 100 mg/kg, p<0.05) treatment groups. Importantly, only one Z944 treated animal reached the fully kindled state. (FIG. 9C) There was no significant difference in average seizure duration between the three treatment groups. *p<0.05, **p<0.01, ****p<0.0001 Z944 vs vehicle. #p<0.05, ##p<0.01 Z944 vs ETX. (FIG. 9A) and (FIG. 9C) Two way repeated measures ANOVA with Bonferroni post-hoc test. (FIG. 9B) One-way ANOVA with Bonferroni post-hoc test.

### Example 4: Post-Status Epilepticus Model Experiments

The post-status epilepticus (post-SE) model closely mimics the epileptogenic processes seen in human TLE. The initial insult is SE induced by kainic acid (KA) the administration of a chemoconvulsant a cyclic analog of L-glutamate and an agonist of ionotropic glutamate AMPA and kainate receptors to cause sustained neuronal depolarization which elicits seizures. The motor seizures are stopped after a period of time (generally >1 hour) by an anticonvulsant such as diazepam. Then, a cascade of changes representative of the human latent period of at least 3-4 weeks culminates in the development of spontaneous seizures. Importantly, the morphological changes that occur in the hippocampus following status epilepticus are similar to those seen in human patients with TLE.

### Preparation of the Osmotic Pumps.

Pumps were filled with either Z944 to deliver a daily dose of 60 mg/kg, levetiracetam to deliver a daily dose of 200 mg/kg or vehicle. For the drug tolerability study, osmotic pumps continuously either Z944, levetiracetam or vehicle for 7 days and for the post-SE study osmotic pumps delivered continuously the different treatments for 28 days.

### Drug Tolerability Study

The neurological side effects of the prolonged administration of Z944 and levetiracetam were assessed over one-week. Naïve wistar rats aged 11 weeks were implanted with a seven-day osmotic pump with Z944 (60 mg/kg/day, n=6), levetiracetam (200 mg/kg/day, n=5) and vehicle (n=6). The animals were evaluated twice per day, morning and afternoon, for side effects from the day of pump implantation to 8 days after.

### FIG. 10. Z944 Shows Minimal and Transient Neurological Side Effects.

Naïve rats receiving Z944 had transient and minimal side effects in comparison to vehicle from day 2 to day 5. Z944 treated animals had significantly reduced neurological side effects during day 2 and 3 when compared to levetiracetam treated animals (p<0.05). Two-way ANOVA with Bonferroni post hoc test. Data shows mean+S.E.M.

### Post-Status Epilepticus Experimental Protocol

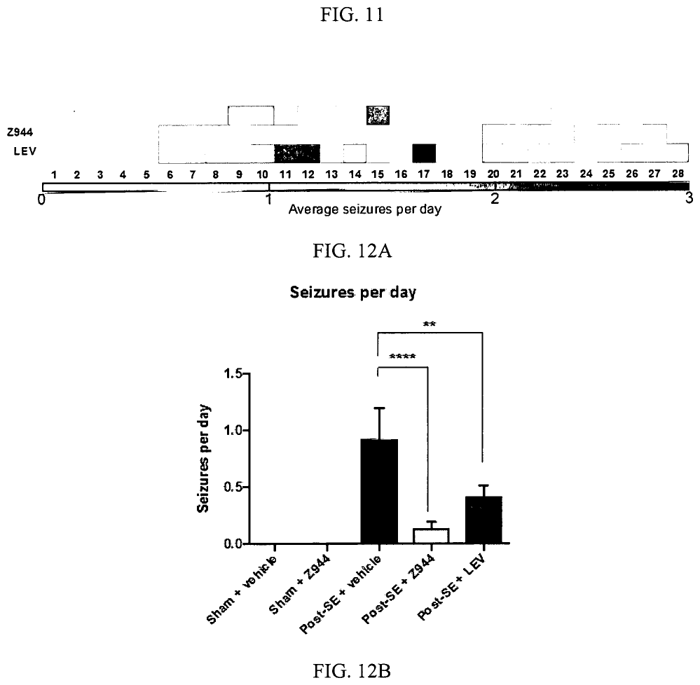

The KA induced post-SE model was used to evaluate the anti-epileptogenic effects of Z944. FIG. 11 summarizes the different methods used on this experimental paradigm.

### FIG. 11. Drug Treatment Protocol in the Post Status Epilepticus Model of Acquired Epilepsy.

KA, kainic acid; SE, status epilepticus; i.p intraperitoneal; EEG, electroencephalogram; wk, week.

### Kainic Acid Induced Post-Status Epilepticus

Animals were implanted with subdural EEG recording electrodes and one week after surgery, the animals were connected using cables that allowed them free movement around their cage. A repeated low dose KA administration protocol was used. Rats were injected i.p. with an initial dose of KA 7.5 mg/kg plus 3 ml of normal saline to prevent dehydration. Animals were monitored for 45 minutes for EEG and behavioural seizures based on the Racine scale. If no self-sustained EEG seizure activity was observed with at least one class IV-V seizure of Racine, another i.p. dose of 2.5 mg/kg of KA was administered. This was repeated up to 2 times every 45 minutes. An animal was eliminated from the experiment if it didn't show a stable self-sustained SE after a maximum KA total dose of 15 mg/kg. Two sham groups were treated identically but received saline injections instead of KA. After four hours of sustained electroencephalographic and behavioural seizures the animals were randomly allocated to one of the five treatment groups: sham+vehicle (n=6), sham+Z944 (n=6), post-SE+vehicle (n=8), post-SE+Z944 (n=8), post-SE+levetiracetam (n=9).

Animals were anesthetized and implanted with subcutaneous osmotic pumps containing either vehicle Z944 (60 mg/kg/day), or levetiracetam (200 mg/kg/day) that delivered the treatment continuously for 4 weeks. Immediately after the pump implantation, SE was stopped with diazepam (5 mg/kg/dose). It is important to note that only transient and minor neurological adverse effects were seen after the prolonged treatment with Z944 or levetiracetam, similar to the drug tolerability studies.

### Video-EEG Recordings and Seizure Analysis

vEEG recordings commenced at least 4 h prior the induction of SE to provide baseline recordings. The video-EEG recordings continued for the whole duration of the treatment period, i.e. four weeks after the termination of the SE. Then, animals were disconnected and did not have vEEG recordings for four weeks (drug wash-out period) and this was followed by another two-week period of vEEG recordings. vEEG recordings were acquired using Profusion 3 software (Compumedics, Australia) unfiltered and digitized at 512 Hz. EEG analysis was performed in a blinded manner and confirmed by two different observers. All EEG recordings were visually and manually annotated using Profusion 3 software. A seizure was defined as an episode of rhythmic spiking activity that was three times the baseline amplitude and a frequency >5 Hz that lasted at least 10 s. The end of a seizure was determined as the last spike. The average number of seizures per day, average seizure duration and seizure class (severity) were analysed.

### Behavioural Tests

All behavioural tests were performed in a light-controlled (˜110 lux), closed, quiet and clean room in the. Behavioural testing was performed in a blinded manner to the treatment groups. All of the behavioural tests were video recorded for future analysis. OF, EPM and MWM sessions were filmed using a digital camera mounted above the centre of the arena, which was connected to a computer for the recording and objective analysis of digitized behavioural tests (EthoVision 3.0.15, Noldus). The first behavioural test was performed one week after the last EEG recording. The different behavioural tests/session were performed 24 hours apart from each other. All tests were scored by an experienced observer who was blind to the experimental conditions of the animals.

### Sucrose Preference Test

The SPT was used to assess depressive-like behaviour. The animals remained to stay in their same home cage. One hour before the start of the test, the animals were given up to 0.5 ml of the testing 2% sucrose to ensure that the animals would drink the sucrose solution. The animals were presented with two bottles, one filled with water and the other one filled with 2% sucrose in water for 24 hours. On both tests, bottle position was randomised to avoid position preference. Total fluid intake and percentage preference for sucrose were recorded.

### Elevated Plus Maze

The EPM is a custom made black acrylic arena in the shape of a plus, with one opposing pair of arms enclosed by 30 cm high walls (closed arms) and the other opposing arms without any enclosure (open arms). Each of the arms is 13 cm wide and 43.5 cm long, and the maze is elevated 60 cm above the floor. The rat was placed in the centre of the EPM, and its behaviour was monitored over 5 min. The EPM was wiped clean between each animal. The distance travelled, number of entries into the open and closed arms, and the total time spent in each arm during the trial were recorded.

### Open Field

The OF is a 100 cm diameter circular arena, with an inner circle arena of 66 cm of diameter that was virtually defined using the Ethovision software. For each test, the rat is placed gently into the centre of the field and its behavioural activity is monitored for 10 min. The distance travel as well as the entries and time spent in the inner circle were recorded.

### Morris Water Maze

The MWM is a circular black PVC plastic pool of 160 cm diameter which is filled to a depth of either 2.5 cm above a 10 cm diameter hidden platform water at 25±1° C. The platform is located in the centre of the South-West quadrant during the first 3 sessions and changed to North-East quadrant for the remaining sessions. Visual cues are placed in the main 4 cardinal points, consisting of large black and white symbols that serve as orientation landmarks for the rats. A trail begins by placing gently the rat in the pool adjacent to, and facing, the pool wall, and ended when the rat stands on the platform or 90 seconds are past. Each trial began at one of four pool walls start locations (North, South, East, or West) according to a pseudo-random schedule of start locations that prevented repeated sequential starts from the same location. Each session consisted of 4 trials. Once session was performed between 8-10 am and the afternoon session between 4-6 pm. The latency to find the escape platform, speed and strategic path used by the animal to locate the platform are recorded. Search time was used as measures of spatial orientation and memory. Swim speed (cm/s) was used as a measure of motor ability.

### Forced Swim Test

The FST was performed in a clear Plexiglas cylinder (50 cm long×30 cm diameter) filled with water (25±1° C.). The FST was divided into a 15-min training session and a 5-min test session performed 24 h later, as previously described. Immobility time and swimming time were analysed.

### Quantitative PCR

RNA was extracted using the RNeasy Mini Kit (QIAGEN), DNA elimination columns were used to remove any genomic DNA contamination in accordance with the manufacturer's protocol. RNA concentration and purity was determined using spectrophotometric readings with the NanoDrop 3000 spectrophotometer (NanoDrop Technologies). 1 μg RNA was reverse transcribed to cDNA using the Omniscript RT Kit (QIAGEN) following the manufacturer's protocol. T-type Ca²⁺ channel expression was assessed using quantitative polymerase chain reaction (qPCR). Each 10 μl reaction volume consisted in 25 ng of cDNA, 5 μl of TaqMan Universal PCR Master Mix 2× and custom designed TaqMan using catalogued 20× Taqman gene expression assays for Ca_{V}3.1 (Assay ID Rn00581051_m1, Applied Biosystems), Ca_{V}3.2 (Assay ID Rn01460348_m1, Applied Biosystems), Ca_{V}3.3 (Assay ID Rn01505208_m1, Applied Biosystems). Relative mRNA expression of the Ca_{V}3.1, Ca_{V}3.2 and Ca_{V}3.3 were compared to the geometric average of the mRNA levels of the housekeeping genes Rplp1 (Rn03467157_gH), Rpl13a (Rn00821946_g1) and Ywhaz (Assay ID: Rn00755072_m1). Analysis was performed using the _{ΔΔ}CT method. The average of the relative expression levels was compared between the different treatment groups.

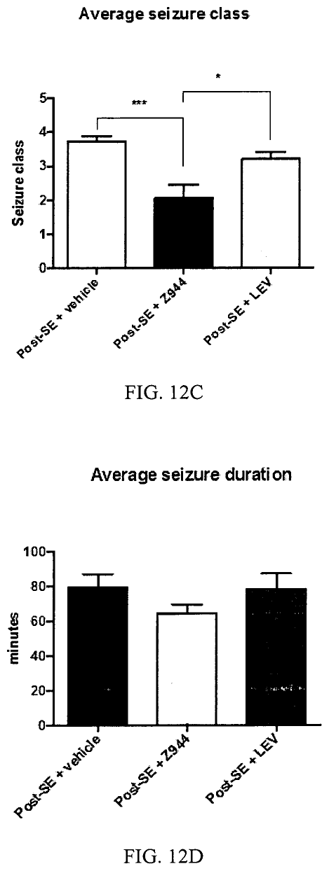

FIGS. 12A, 12B, 12C, 12D. Z944 Treatment has Anti-Epileptogenic Effects after SE. Data Shows the Analysis of EEG During the 4 Weeks of Continuous Treatment

(FIG. 12A) heat map shows the average number of seizures per day during the 4 weeks of treatment after post-SE. (FIG. 12B) average seizures per day (FIG. 12C) Average seizure duration and (FIG. 12D) seizure severity. (*p<0.05, **p<0.01, p<0.001,***p<000.01). One-way ANOVA with Bonferroni post hoc. Data shows mean+S.E.M

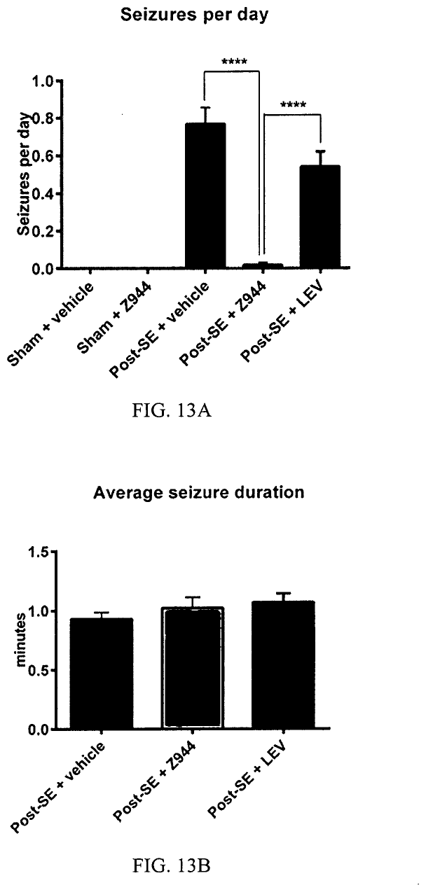

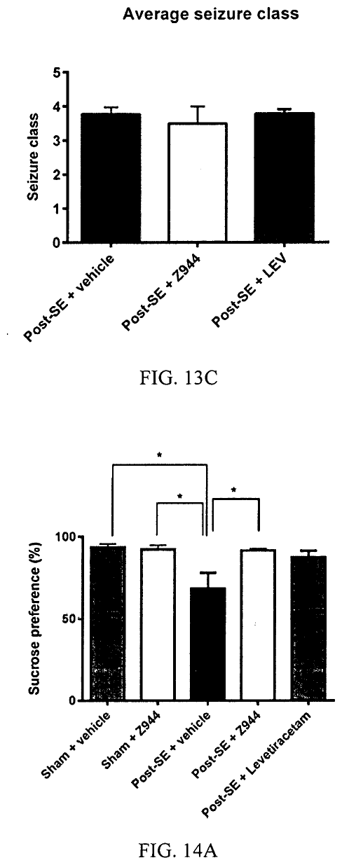

### FIGS. 13A, 13B, 13C. Z944 is Anti-Epileptogenic.

Data shows the analysis of EEG after 4 weeks from drug washout. (FIG. 13A) average number of seizures per day. Importantly, only two of the eight Z944 treated animal developed seizures and each of these animals presented only one seizure in two weeks. As expected both of the sham animals did not showed any seizures; (FIG. 13B) seizure duration and (FIG. 13C) seizure severity. (****p<0.0001). One-way ANOVA with Bonferroni post hoc. Data shows mean+S.E.M.

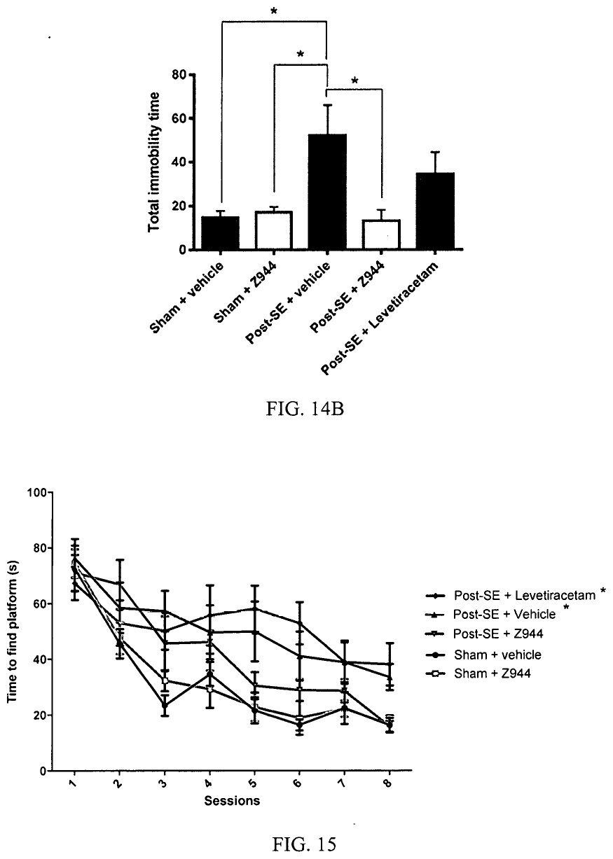

FIGS. 14A, 14B. Z944 Treatment after Status Epilepticus Reduced Depressive Like Behaviour

(FIG. 14A) the sucrose preference test. Post-SE animals treated with vehicle had a reduced sucrose preference compared to both sham groups and post-SE+Z944 group (*p<0.05) and (FIG. 14B) the forced swim test, post-SE animals treated with vehicle had an increased immobility time when compared to both sham groups and post-SE+Z944 group (*p<0.05). One-way ANOVA with Bonferroni post hoc. Data shows mean+S.E.M.

FIG. 15. Z944 Treatment after Status Epilepticus Improves Cognition in Rats.

Vehicle and levetiracetam treated animals after SE took significantly longer to find the hidden platform than shams and treated animals. Post-SE+vehicle and post-SE+levetiracetam when compared to shams and post-SE treated with Z944 (*p<0.05 for all the comparisons. Two-way ANOVA with Bonferroni post hoc test. Data shows mean+S.E.M.

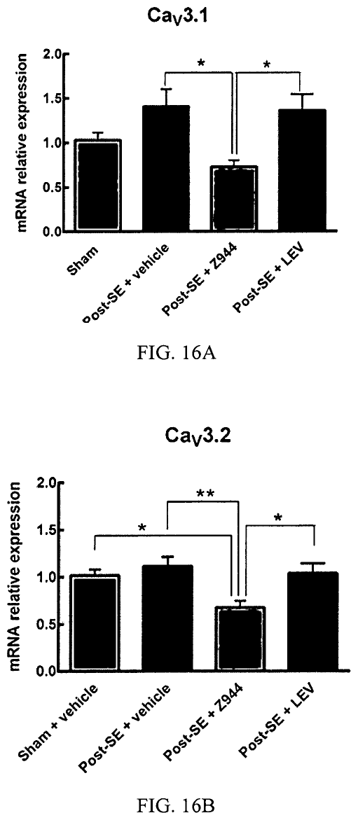

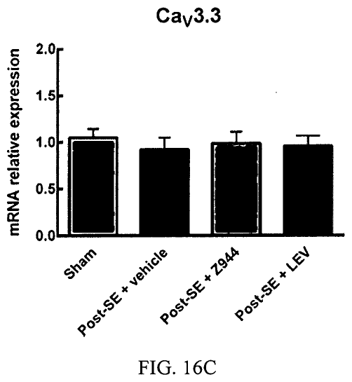

FIGS. 16A, 16B, 16C. Z944 Treatment after Status Epilepticus Reduced mRNA Expression of T-Type Calcium Channels in the Hippocampus 9 Weeks after Treatment Ended.

(FIG. 16A) Cav 3.1 mRNA expression (FIG. 16B) Cav 3.2 mRNA expression and (FIG. 16C) Cav 3.3 mRNA expression (*p<0.05, **p<0.01). Post-SE, post-status epilepticus. One-way ANOVA with Bonferroni post hoc test. Data shows mean+S.E.M.

### Example 5: Administration of Exemplary Compounds as an Anti-Epileptic Treatment for Drug Resistant Absence Seizures in Adults (Phase Ib)

Here we propose a Phase Ib dose ranging study for early stage clinical development of Z944 as a new treatment for adults with drug resistant absence seizures.

Participant Population:

10 Subjects ≥16 years of age with IGE and ongoing, drug resistant absence seizures (at least 1 per day on average, at least 13-4 Hz SWD lasting at least 5 seconds) taking at least one AED.

Study Design Outline:

Weekly dose escalation study. Subjects will receive a baseline 3 hour (9 am-12 pm) video-EEG recording to quantify the amount of spike and wave discharges and absence seizures that the patient is having, and will also undergo hyperventilation (HV) and photic stimulation (PS) challenge. They will then start treatment with Z944 (extended release formulation) at 20 mg twice a day for a week. During this week subjects would be asked to record in a diary (e.g., on a smartphone) their seizure occurrences and adverse effects. After this subjects would return for a further 3 hour video-EEG recording (include HV and PS challenge) and blood samples are obtained for PK and safety data, and then the dose of Z944 increased to 40 mg twice a day taken for another week (with i-phone diary their seizure occurrences and adverse effects). This cycle would then be repeated again a week later and the dose escalated to 60 mg BD, and then again a week later to 80 mg BD for a week (if tolerated).

The primary endpoints would be the total time in SWDs and in seizures on the video-EEG recordings, and the adverse events. The secondary endpoint would be patient reported seizures and any decrease in SWDs with HV and PS challenge. These outcomes would inform the dose to be used in the Phase IIa RCT.

### Example 6: Administration of Exemplary Compounds as an Anti-Epileptic Treatment for Drug Resistant Absence Seizures in Adults (Phase IIa)

Here we propose a Phase IIa RCT trial of Z944 to treat adults with drug resistant absence seizures. The trial will be aimed at evaluating efficacy and safety/tolerability for of Z944 treatment over two months, and will also provide a pilot evaluation of persisting disease modifying effects of the Z944 treatment.

Participant Population:

40 Subjects ≥16 years of age with IGE and ongoing, drug resistant absence seizures (at least 1 per day on average) taking at least one AED.

Study Design Outline:

Subjects would have two baseline 3 hour video-EEG recording two weeks apart, and keep two weeks of seizure diaries (e.g., on a smartphone). They will then be randomised to treatment in a double-blinded fashion with add-on Z944, at the optimal dose as determined in the Phase Ia study, or placebo for 8 weeks after a 2 week escalation phase. 3 hour video-EEG recordings will be acquired after the dose escalation phase, and then again after 4 and 8 weeks of the maintenance phase. Blood samples will be taken for PK and safety data at each video-EEG recording visit. After completion of the treatment phase further three hour video-EEG recordings are obtained at 4 weeks and 12 weeks to assess whether there has been a sustained post-treatment suppression in seizures, indicating a disease modifying effect.

The primary endpoints to be compared between Z944 and placebo treated patients would be the total time in SWDs and in seizures on the video-EEG recordings, and the adverse events. The secondary endpoint would be patient reported seizures.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of treating absence seizures in a subject in need thereof, wherein the method comprises orally administering to the subject a compound selected from: 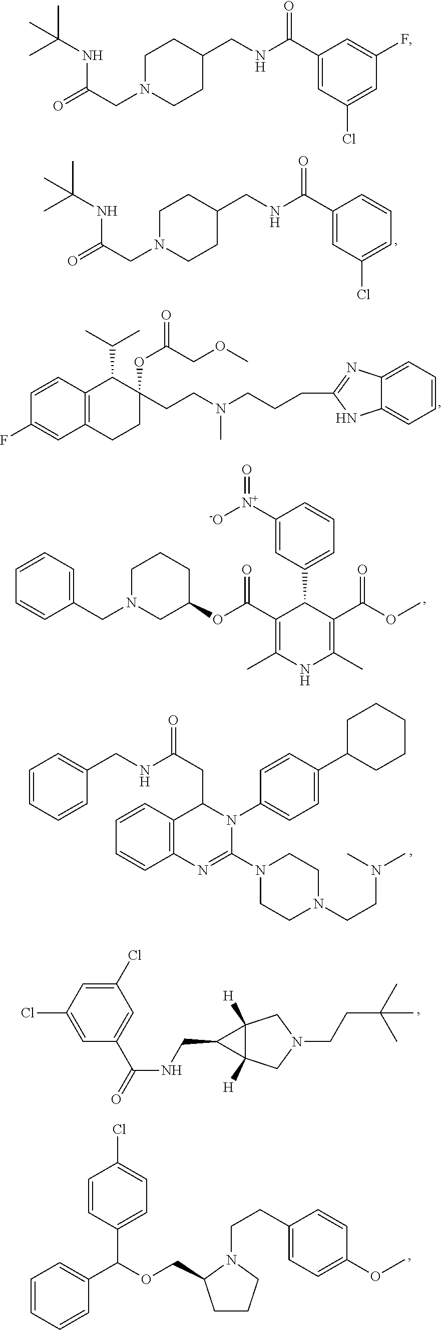 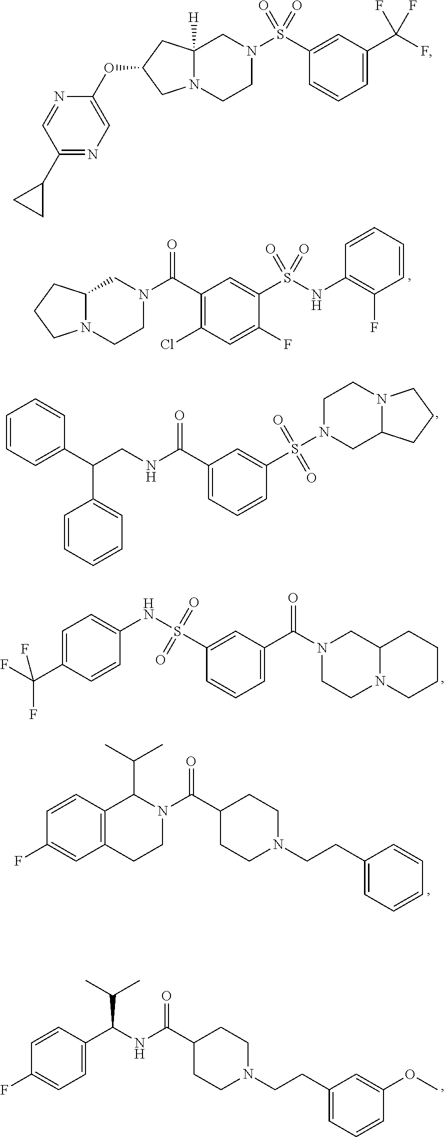 [SMILES: O=S(=O)(c1cccc(C(F)(F)F)c1)N1CCN2C[C@H](Oc3cnc(C4CC4)cn3)C[C@H]2C1 ; O=C(c1cc(S(=O)(=O)Nc2ccccc2F)c(F)cc1Cl)N1CCN2CCC[C@@H]2C1 ; O=C(NCC(c1ccccc1)c1ccccc1)c1cccc(S(=O)(=O)N2CCN3CCCC3C2)c1 ; O=C(c1cccc(S(=O)(=O)Nc2ccc(C(F)(F)F)cc2)c1)N1CCN2CCCCC2C1 ; CC(C)C1c2ccc(F)cc2CCN1C(=O)C1CCN(CCc2ccccc2)CC1 ; COc1cccc(CCN2CCC(C(=O)N[C@@H](c3ccc(F)cc3)C(C)C)CC2)c1] 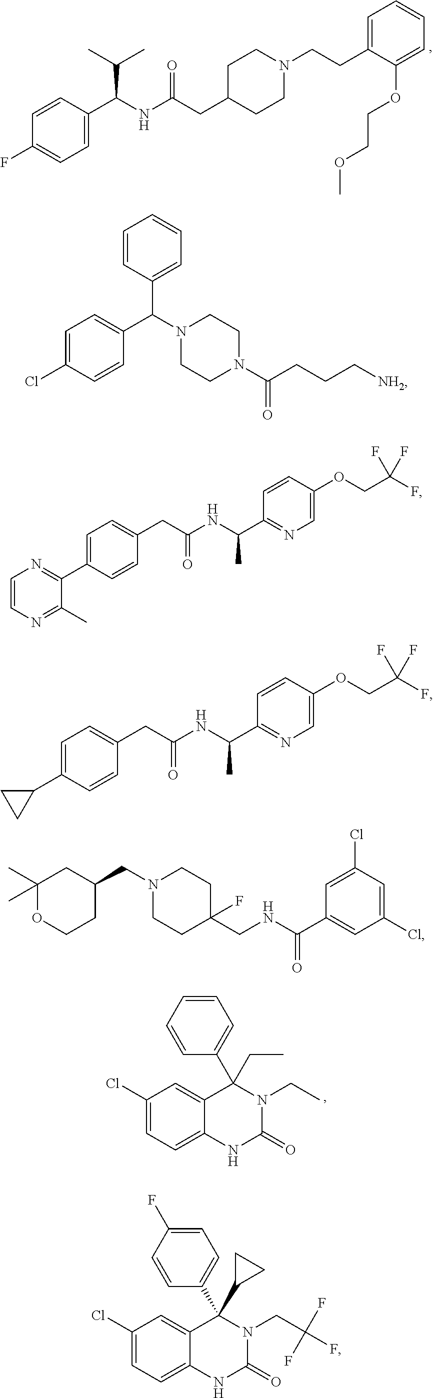 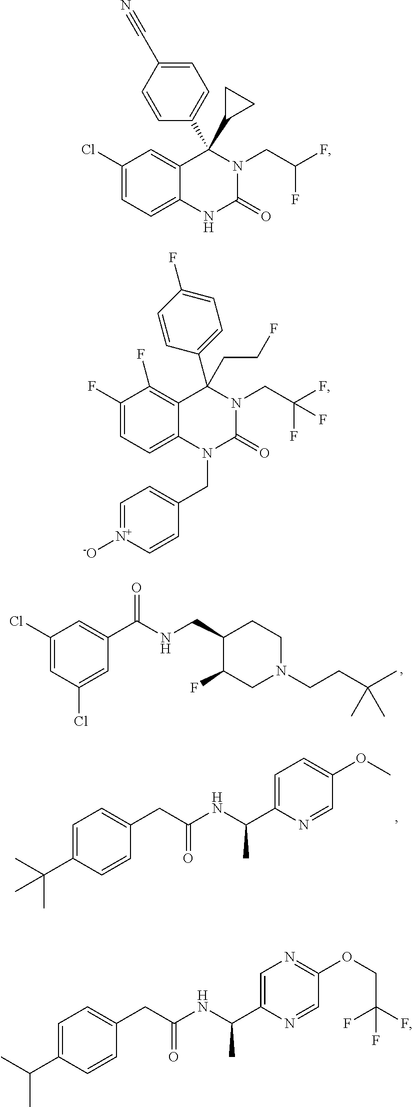 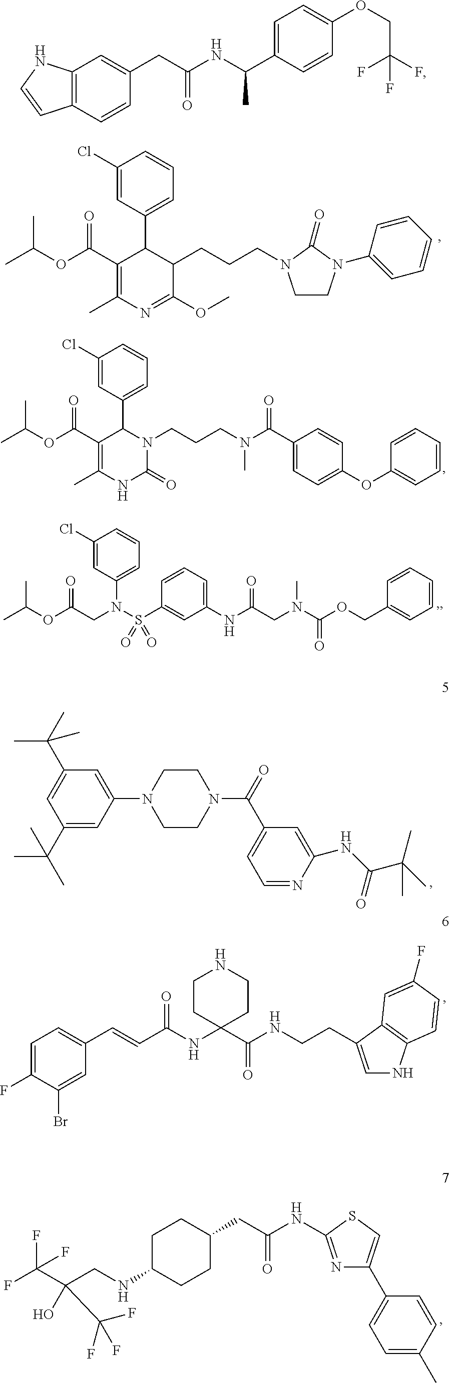 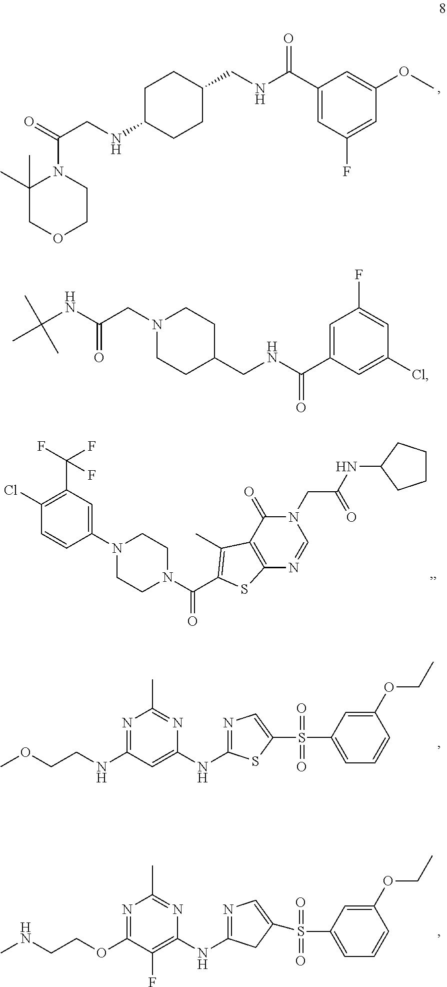 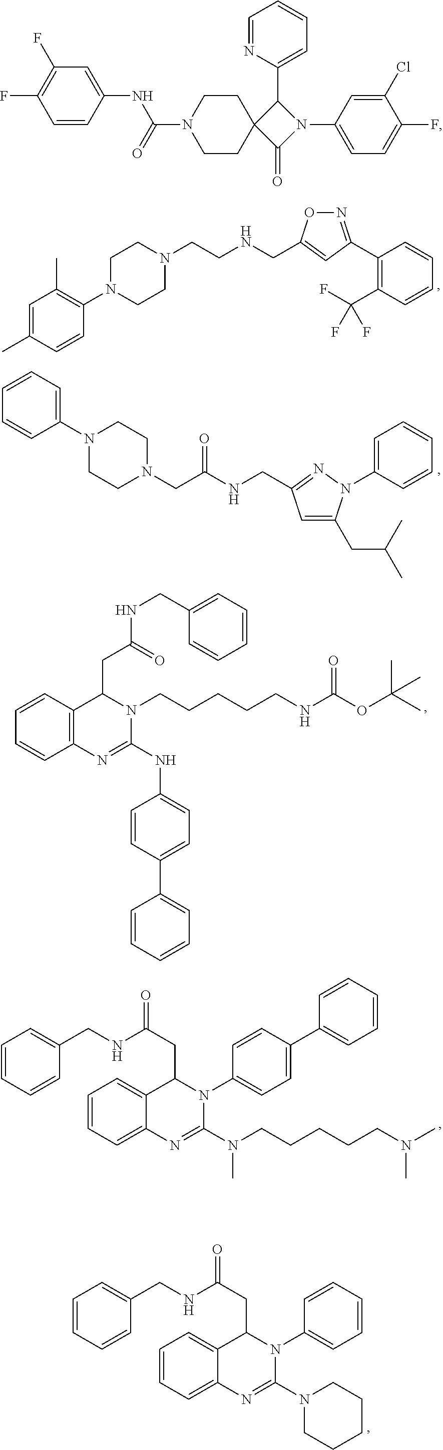 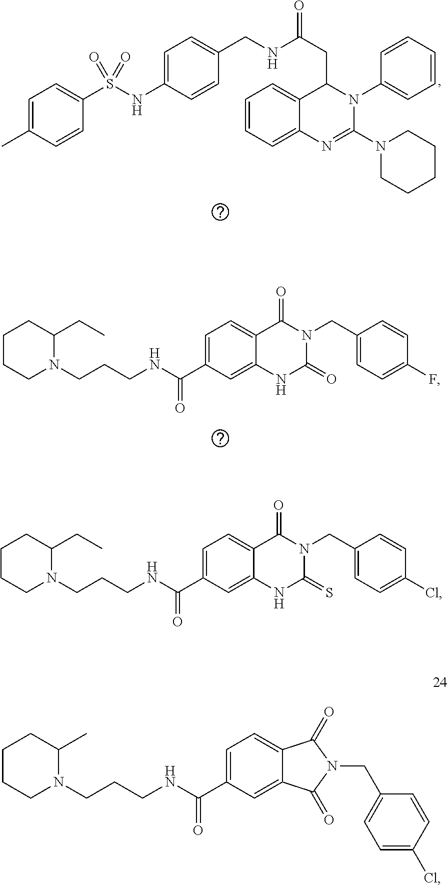 [SMILES: Cc1ccc(S(=O)(=O)Nc2ccc(CNC(=O)CC3c4ccccc4N=C(N4CCCCC4)N3c3ccccc3)cc2)cc1 ; CCC1CCCCN1CCCNC(=O)c1ccc2c(=O)n(Cc3ccc(F)cc3)c(=O)[nH]c2c1 ; CCC1CCCCN1CCCNC(=O)c1ccc2c(=O)n(Cc3ccc(Cl)cc3)c(=S)[nH]c2c1 ; CC1CCCCN1CCCNC(=O)c1ccc2c(c1)C(=O)N(Cc1ccc(Cl)cc1)C2=O] 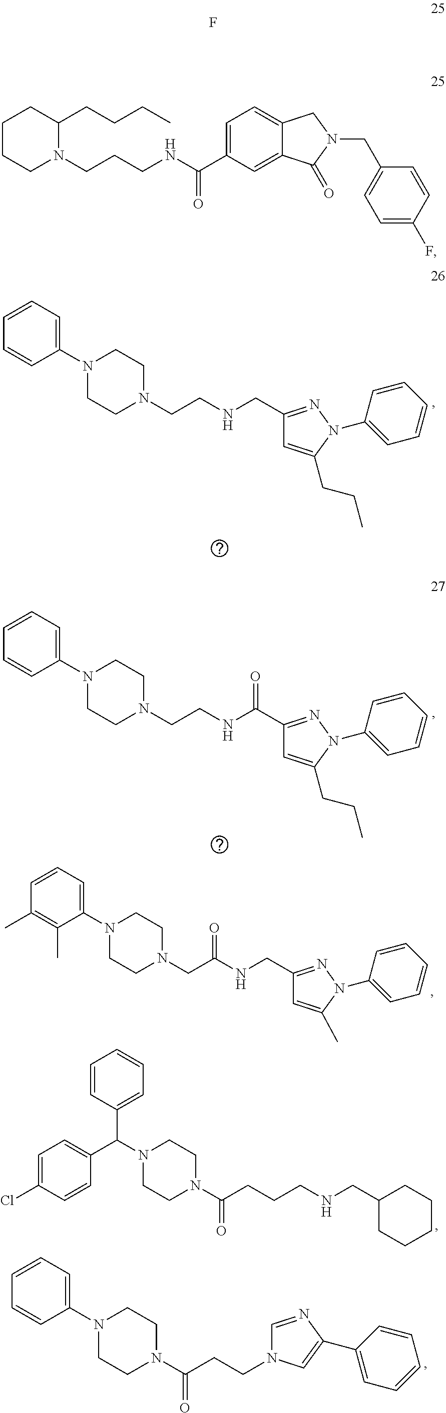 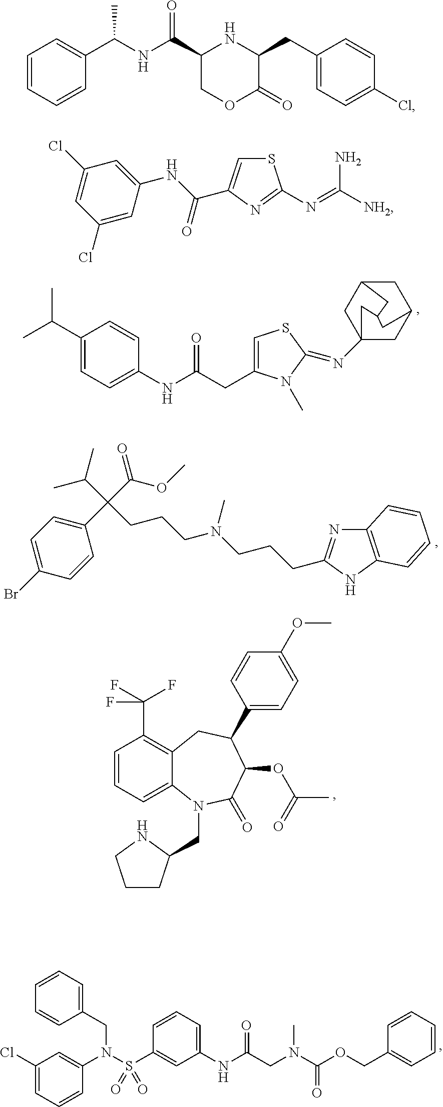 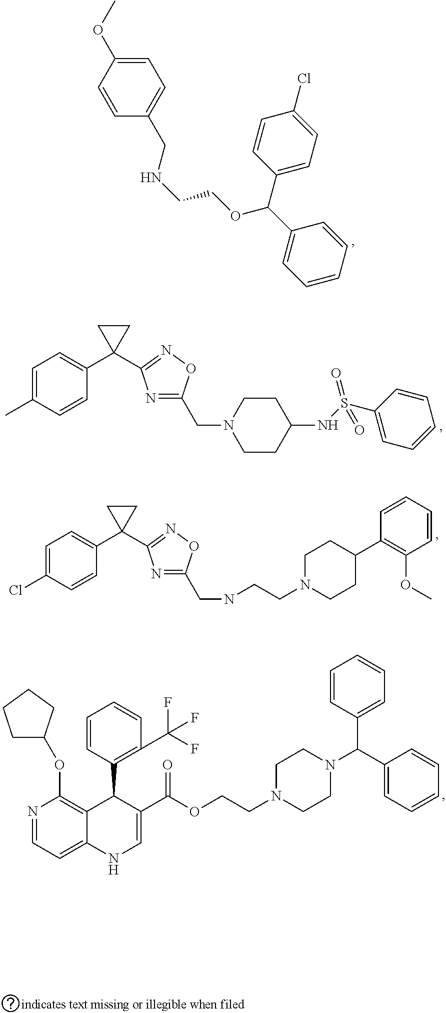
N-[(1R)-1-(4-fluorophenyl)-2-methylpropyl]-1-[2-(3-methoxyphenyl)ethyl]piperidine-4-carboxamide, 6-fluoro-1-isopropyl-2-{[1-(2-phenylethyl)piperidin-4-yl)carbonyl-1,2,3,4-tetrahydroisoquinoline, diltiazem, tylerdipine, P-11520031, DP-3005, RQ-00311610, A-1264087, A-1315647, VMD-3816 or VMD-3222, or a pharmaceutically acceptable salt thereof,
wherein the dosage of the compound is about 10 mg or more; or
the compound is administered concurrently with an anti-epileptic drug (e.g, ethosuximide, valproic acid, or lamotrigine); or
the absence seizures are refractory absence seizures.

2. The method of claim 1, wherein the compound is 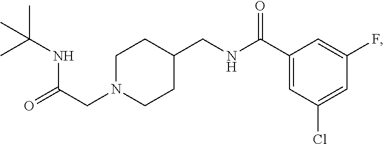 [SMILES: CC(C)(C)NC(=O)CN1CCC(CNC(=O)c2cc(F)cc(Cl)c2)CC1]
or a pharmaceutically acceptable salt thereof.

3. The method of claim 1, wherein the subject experiences at least one seizure per day.

4. The method of claim 3, wherein the subject experiences at least one seizure per day comprising at least 1-6 Hz (e.g., 3-4 Hz) SWD.

5. The method of claim 3, wherein the subject experiences at least one seizure per day lasting about 5 seconds or more (e.g., about 10 seconds or more, about 15 seconds or more, about 20 seconds or more, about 30 seconds or more, or about 1 minute or more).

6. The method of any one of the preceding claims, wherein the subject experiences a decrease in seizure SWDs upon administration of the compound.

7. The method of any one of the preceding claims, wherein the compound is administered concurrently with an anti-epileptic drug (e.g, ethosuximide, valproic acid, or lamotrigine).

8. The method of any one of the preceding claims, wherein the concurrent administration comprises simultaneous administration, or administration of the compound before or after an anti-epileptic drug (e.g., ethosuximide, valproic acid, or lamotrigine).

9. The method of any one of the preceding claims, wherein the absence seizures are refractory absence seizures.

10. The method of any one of the preceding claims, wherein the absence seizures are refractory to an anti-epileptic drug (e.g., ethosuximide, valproic acid, or lamotrigine).

11. The method of any one of the preceding claims, wherein the compound is administered daily.

12. The method of claim 11, wherein the compound is administered twice daily.

13. The method of claim 11, wherein the compound is administered daily for at least one week.

14. The method of claim 11, wherein the compound is administered daily for more than one week.

15. The method of any one of the preceding claims, wherein the dosage of the compound of is greater than 10 mg.

16. The method of any one of the preceding claims, wherein the dosage of the compound is about 20 mg.

17. The method of any one of the preceding claims, wherein the dosage of the compound is about 40 mg.

18. The method of any one of the preceding claims, wherein the dosage of the compound is about 60 mg.

19. The method of any one of the preceding claims, wherein the subject has epilepsy.

20. The method of any one of the preceding claims, wherein the absence seizures are atypical absence seizures.

21. The method of any one of the preceding claims, wherein the absence seizures comprise adult absence seizures, juvenile absence seizures, or childhood absence seizures.

22. The method of any one of the preceding claims, wherein the subject is a mammal (e.g., a human).

23. The method of any one of the preceding claims, wherein the subject is an adult (e.g., male or female).

24. The method of any one of claims 1-22, wherein the subject is a child.

25. The method of any one of the preceding claims, further comprising analyzing or receiving analysis of an EEG recording at least once prior to the end of treatment.

26. The method of claim 25, wherein the dosage is adjusted (e.g., increased) based on analysis of an EEG recording.

27. The method of any one of the preceding claims, further comprising analyzing or receiving analysis of a blood sample from the subject at least once prior to the end of treatment.

28. The method of claim 25, wherein the dosage is adjusted (e.g., increased) based on analysis of a blood sample.

29. The method of any one of the preceding claims, wherein the compound is administered daily for one week at a dosage of 20 mg, then administered daily for a week at a dosage greater than 20 mg (e.g., 25 mg, 30 mg, 35 mg, 40 mg, 45 mg, or 50 mg).

30. The method of any one of the preceding claims, wherein the compound is administered daily for one week at a dosage of 20 mg, then administered daily for a week at a dosage of 40 mg, then administered daily for a week at a dosage greater than 40 mg (e.g., 45 mg, 50 mg, 55 mg, 60 mg, 65 mg, or 70 mg).

31. A method of treating juvenile myoclonic epilepsy in a subject in need thereof, wherein the method comprises orally administering to the subject a compound selected from: 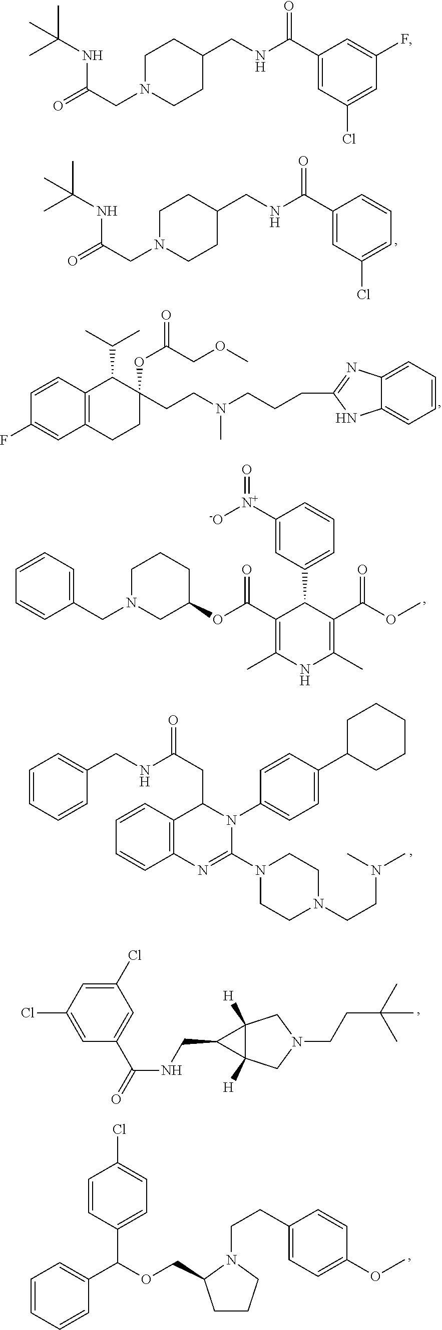 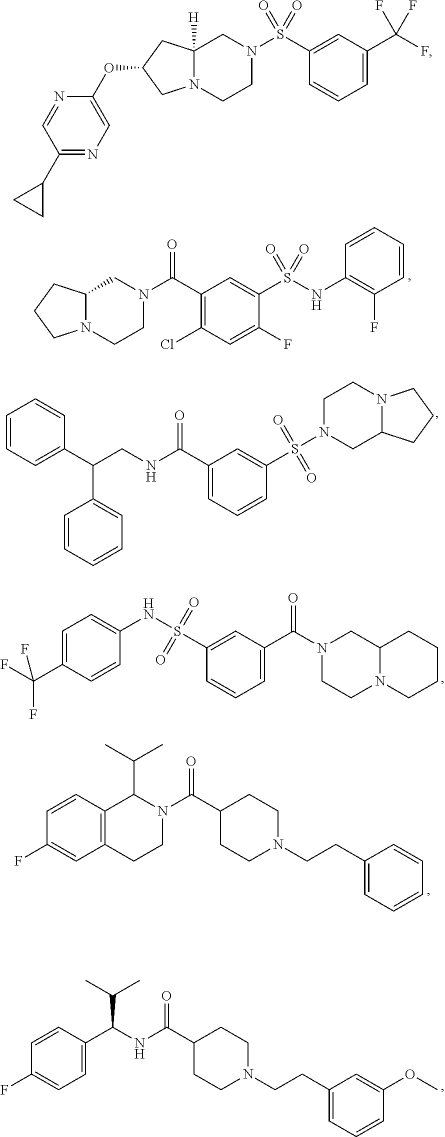 [SMILES: O=S(=O)(c1cccc(C(F)(F)F)c1)N1CCN2C[C@H](Oc3cnc(C4CC4)cn3)C[C@H]2C1 ; O=C(c1cc(S(=O)(=O)Nc2ccccc2F)c(F)cc1Cl)N1CCN2CCC[C@@H]2C1 ; O=C(NCC(c1ccccc1)c1ccccc1)c1cccc(S(=O)(=O)N2CCN3CCCC3C2)c1 ; O=C(c1cccc(S(=O)(=O)Nc2ccc(C(F)(F)F)cc2)c1)N1CCN2CCCCC2C1 ; CC(C)C1c2ccc(F)cc2CCN1C(=O)C1CCN(CCc2ccccc2)CC1 ; COc1cccc(CCN2CCC(C(=O)N[C@@H](c3ccc(F)cc3)C(C)C)CC2)c1] 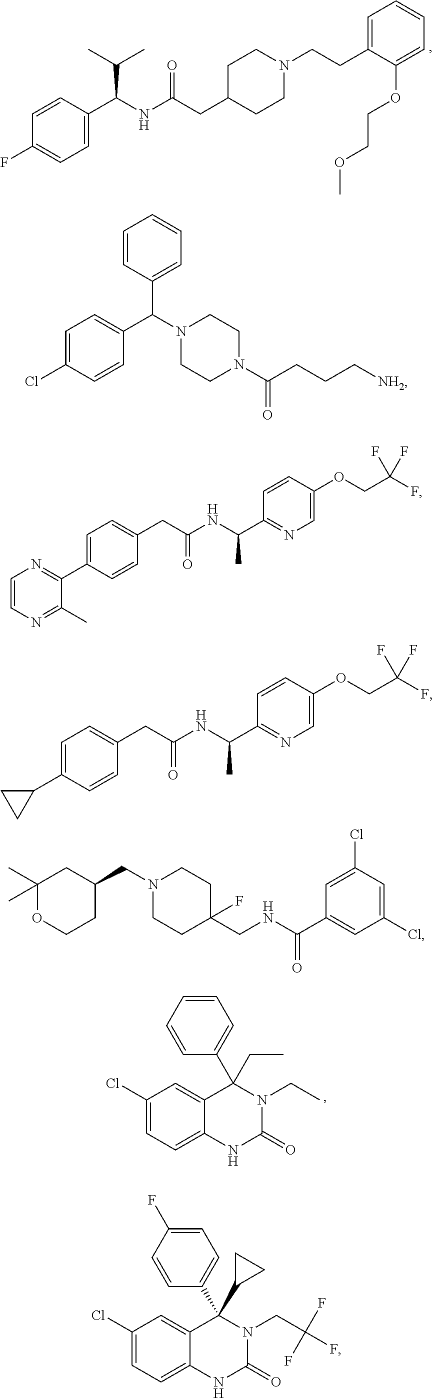 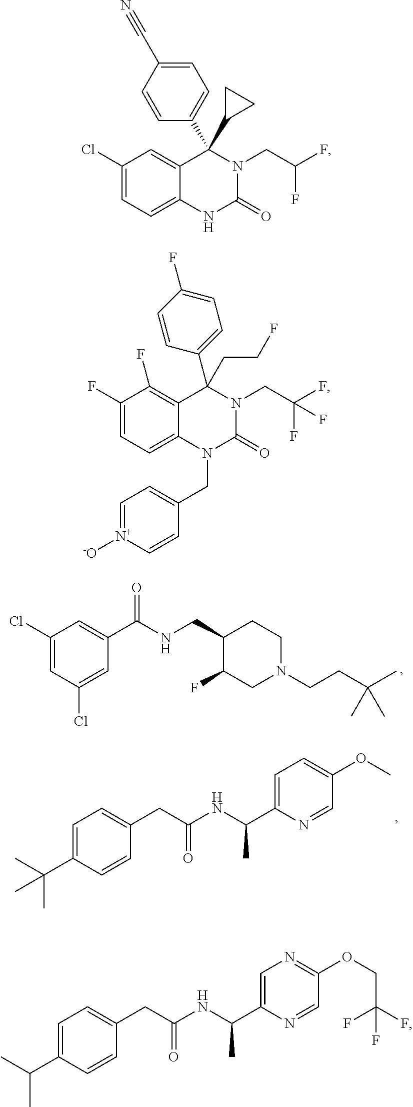 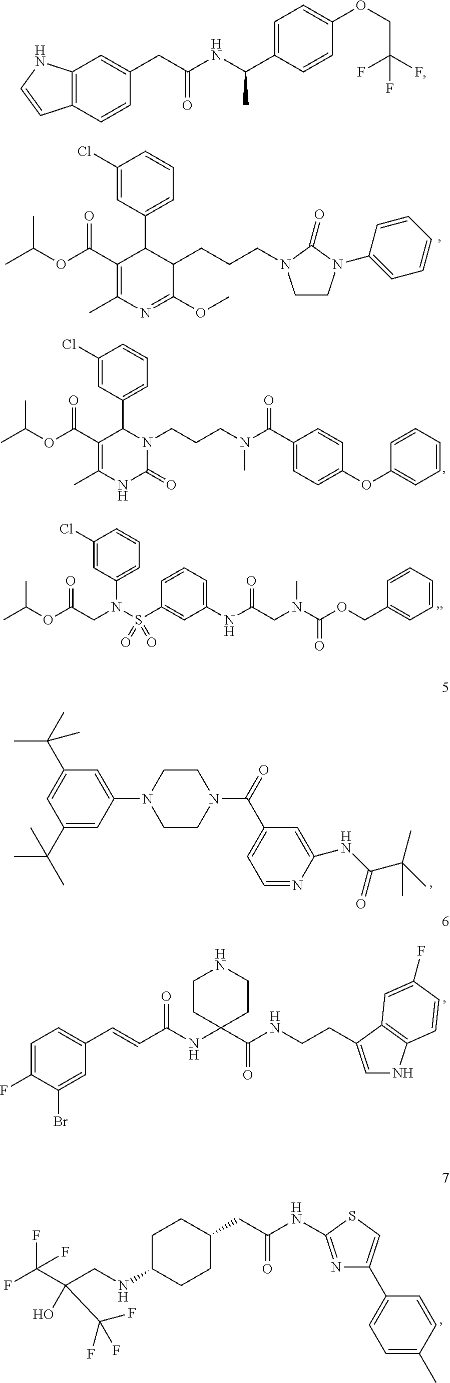 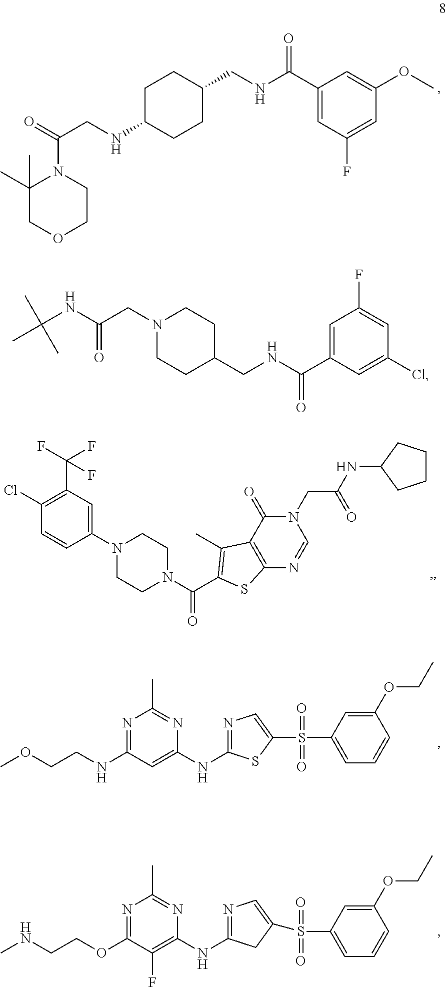 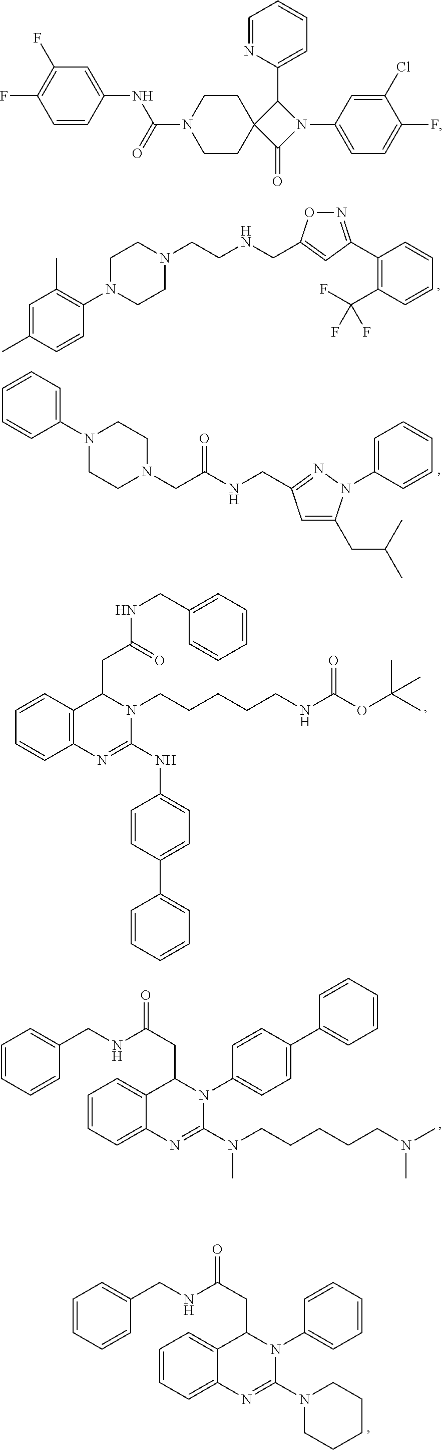 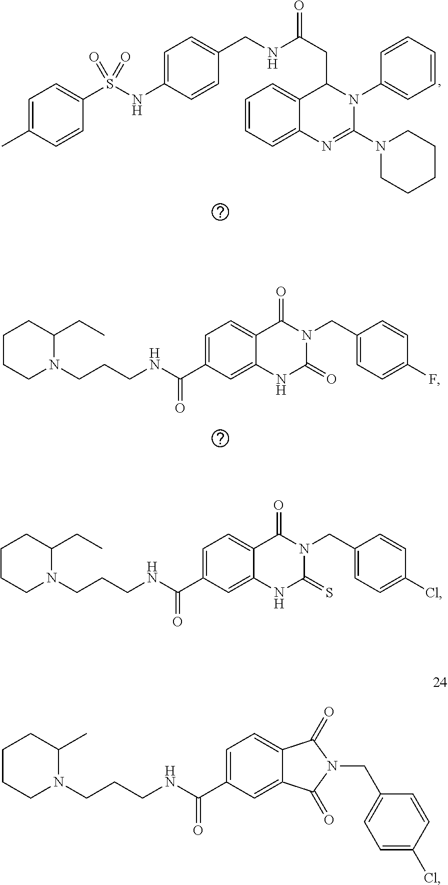 [SMILES: Cc1ccc(S(=O)(=O)Nc2ccc(CNC(=O)CC3c4ccccc4N=C(N4CCCCC4)N3c3ccccc3)cc2)cc1 ; CCC1CCCCN1CCCNC(=O)c1ccc2c(=O)n(Cc3ccc(F)cc3)c(=O)[nH]c2c1 ; CCC1CCCCN1CCCNC(=O)c1ccc2c(=O)n(Cc3ccc(Cl)cc3)c(=S)[nH]c2c1 ; CC1CCCCN1CCCNC(=O)c1ccc2c(c1)C(=O)N(Cc1ccc(Cl)cc1)C2=O] 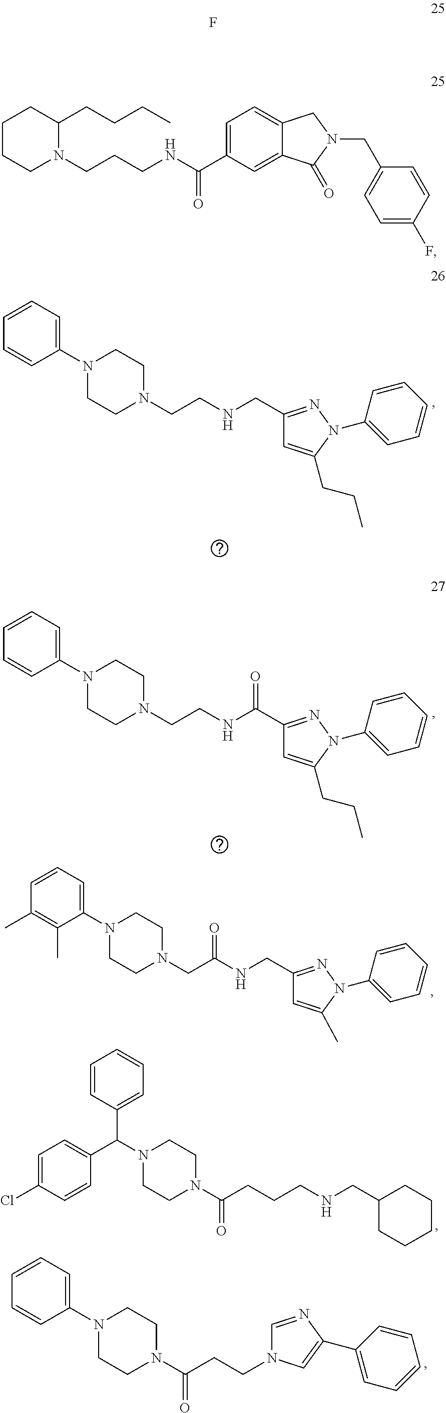 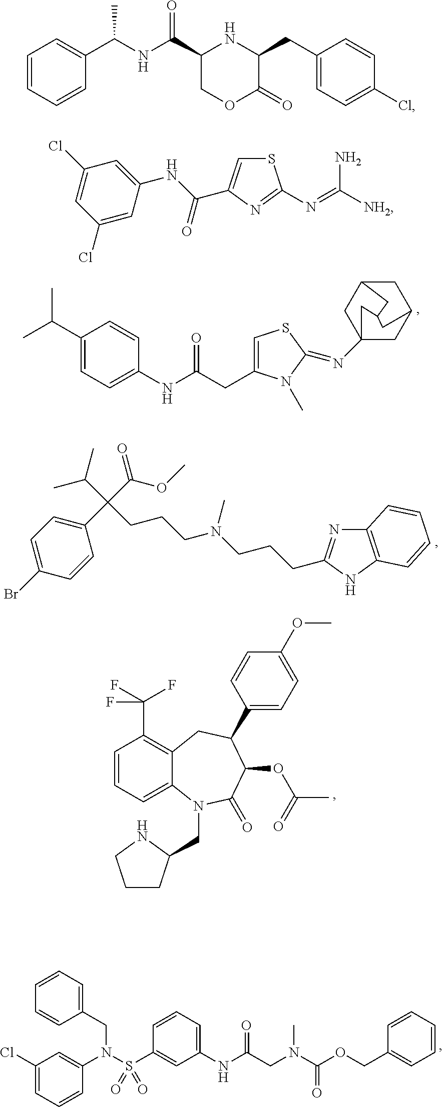 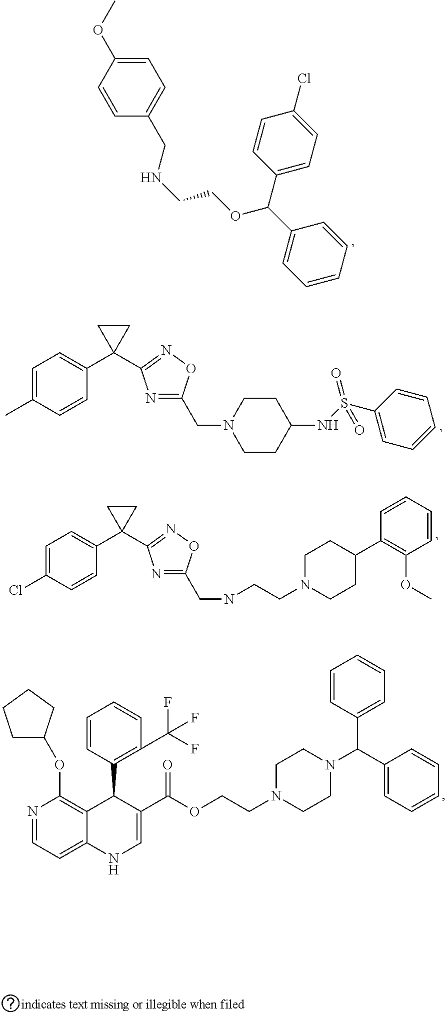
N-[(1R)-1-(4-fluorophenyl)-2-methylpropyl]-1-[2-(3-methoxyphenyl)ethyl]piperidine-4-carboxamide, 6-fluoro-1-isopropyl-2-{[1-(2-phenylethyl)piperidin-4-yl)carbonyl-1,2,3,4-tetrahydroisoquinoline, diltiazem, tylerdipine, P-11520031, DP-3005, RQ-00311610, A-1264087, A-1315647, VMD-3816 or VMD-3222, or a pharmaceutically acceptable salt thereof,
wherein the dosage of the compound is about 10 mg or more; or
the compound is administered concurrently with an anti-epileptic drug (e.g, ethosuximide, valproic acid, or lamotrigine); or
the juvenile myoclonic epilepsy is refractory juvenile myoclonic epilepsy.

32. A method of treating a genetic epilepsy in a subject in need thereof, wherein the method comprises orally administering to the subject a compound selected from: 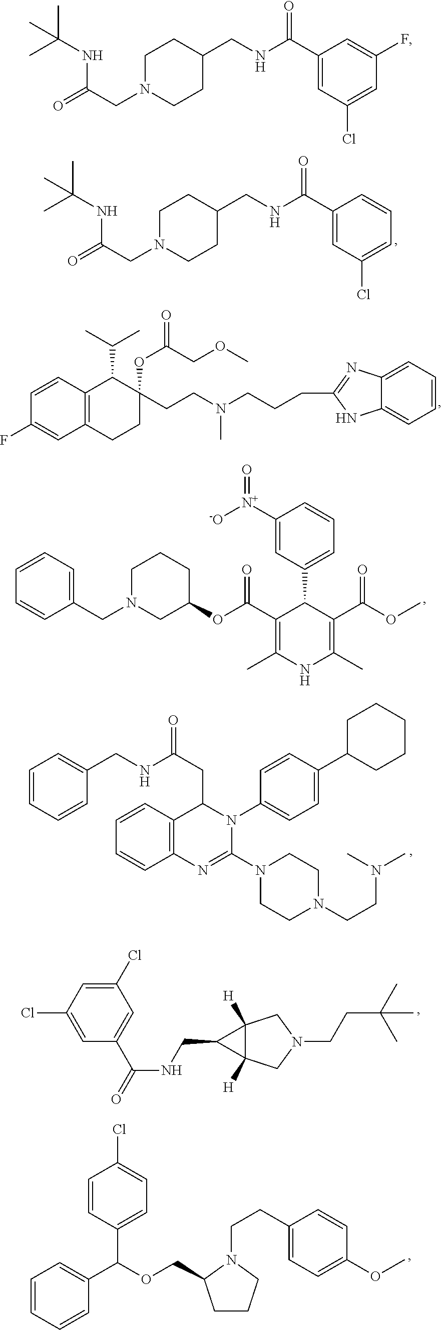 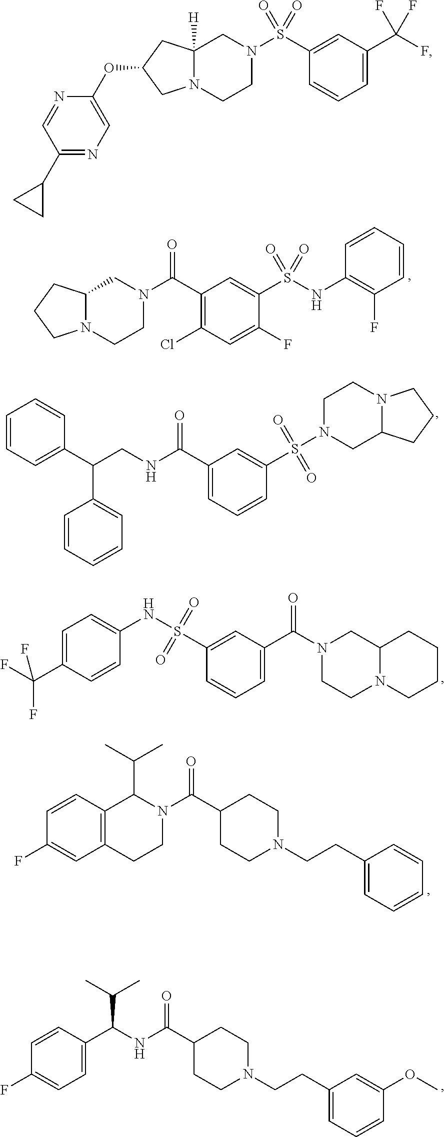 [SMILES: O=S(=O)(c1cccc(C(F)(F)F)c1)N1CCN2C[C@H](Oc3cnc(C4CC4)cn3)C[C@H]2C1 ; O=C(c1cc(S(=O)(=O)Nc2ccccc2F)c(F)cc1Cl)N1CCN2CCC[C@@H]2C1 ; O=C(NCC(c1ccccc1)c1ccccc1)c1cccc(S(=O)(=O)N2CCN3CCCC3C2)c1 ; O=C(c1cccc(S(=O)(=O)Nc2ccc(C(F)(F)F)cc2)c1)N1CCN2CCCCC2C1 ; CC(C)C1c2ccc(F)cc2CCN1C(=O)C1CCN(CCc2ccccc2)CC1 ; COc1cccc(CCN2CCC(C(=O)N[C@@H](c3ccc(F)cc3)C(C)C)CC2)c1] 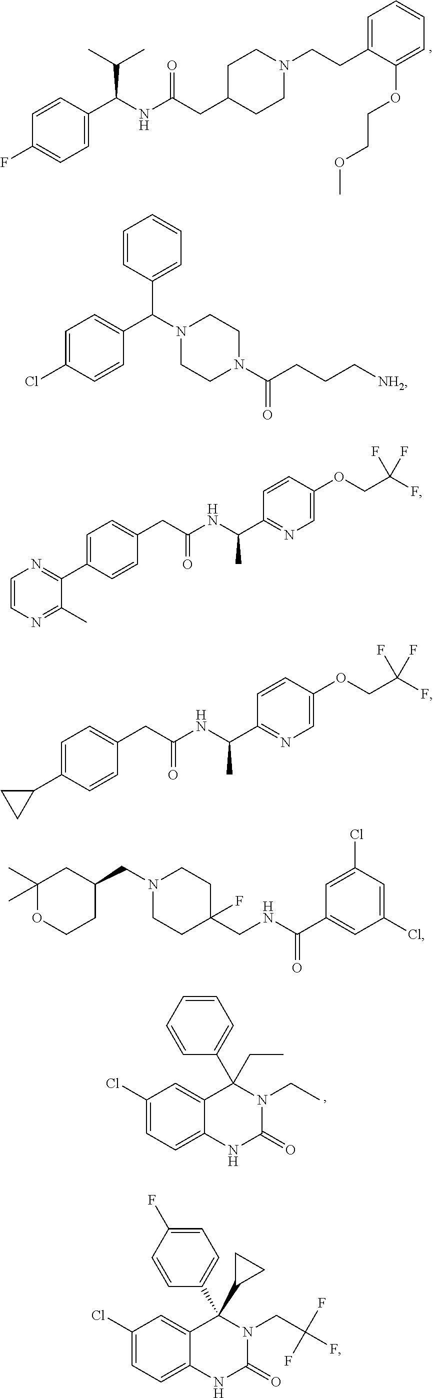 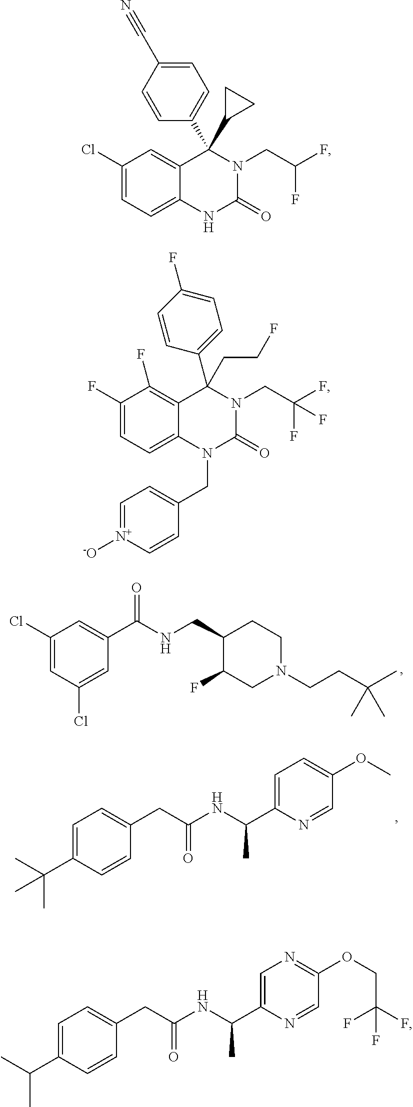 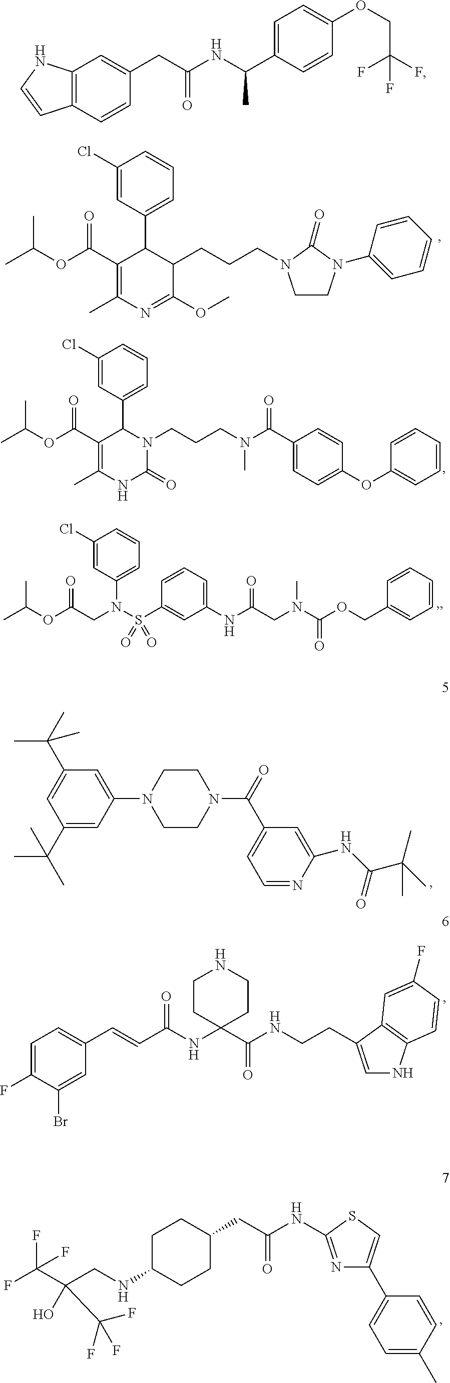 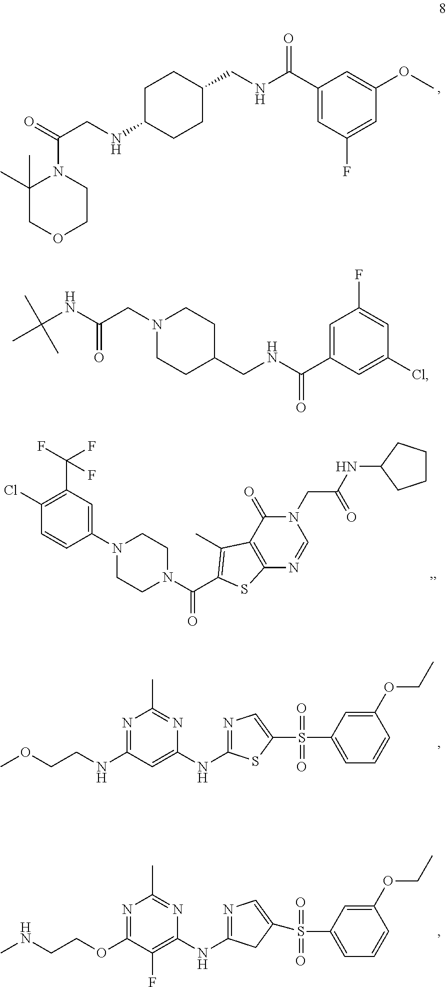 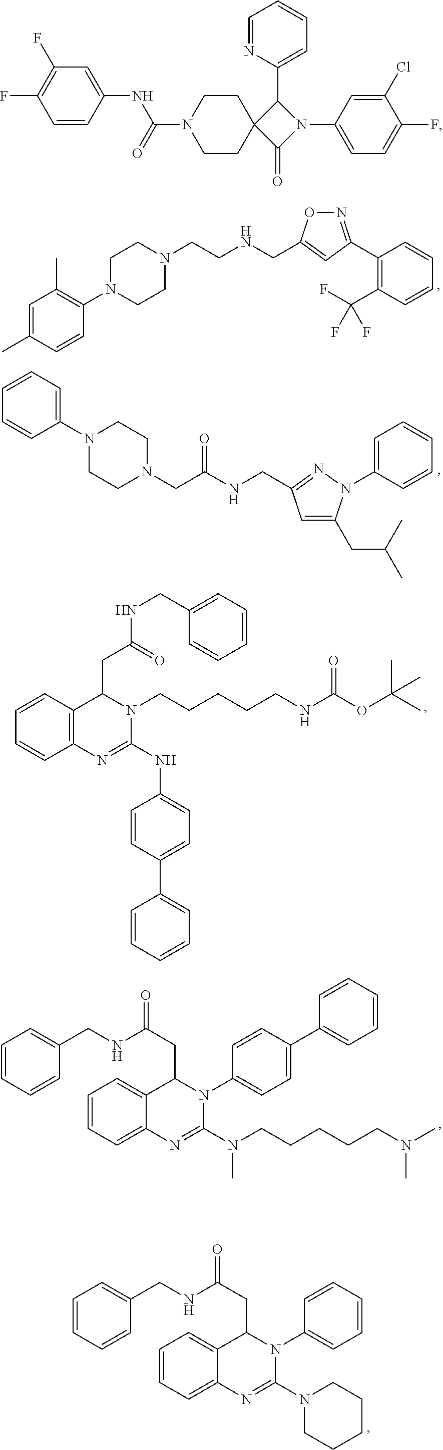 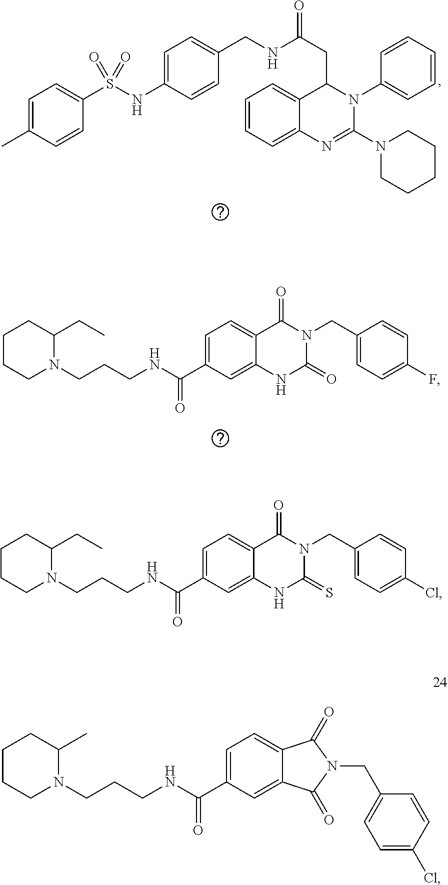 [SMILES: Cc1ccc(S(=O)(=O)Nc2ccc(CNC(=O)CC3c4ccccc4N=C(N4CCCCC4)N3c3ccccc3)cc2)cc1 ; CCC1CCCCN1CCCNC(=O)c1ccc2c(=O)n(Cc3ccc(F)cc3)c(=O)[nH]c2c1 ; CCC1CCCCN1CCCNC(=O)c1ccc2c(=O)n(Cc3ccc(Cl)cc3)c(=S)[nH]c2c1 ; CC1CCCCN1CCCNC(=O)c1ccc2c(c1)C(=O)N(Cc1ccc(Cl)cc1)C2=O] 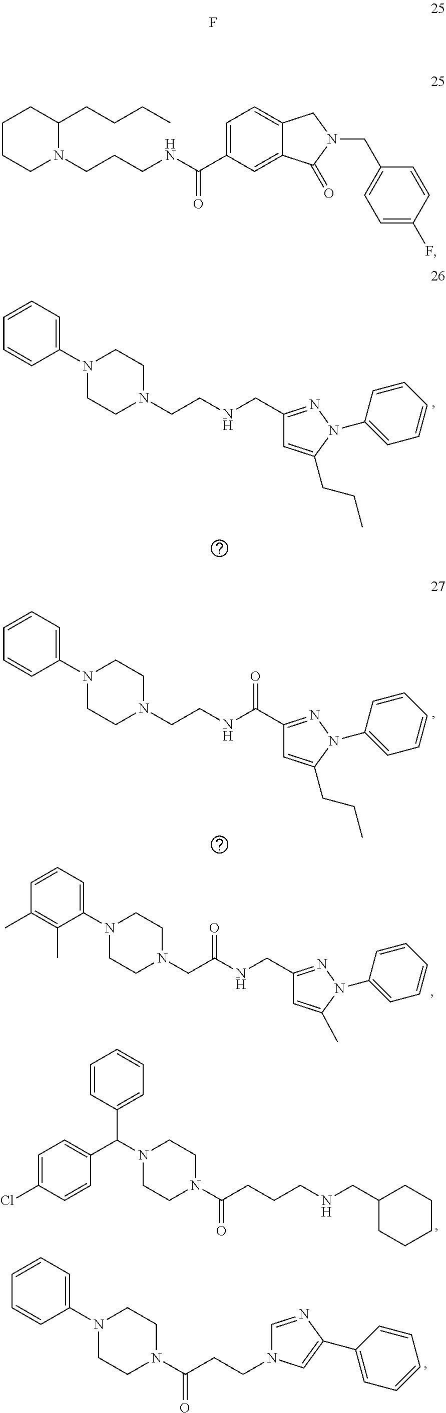 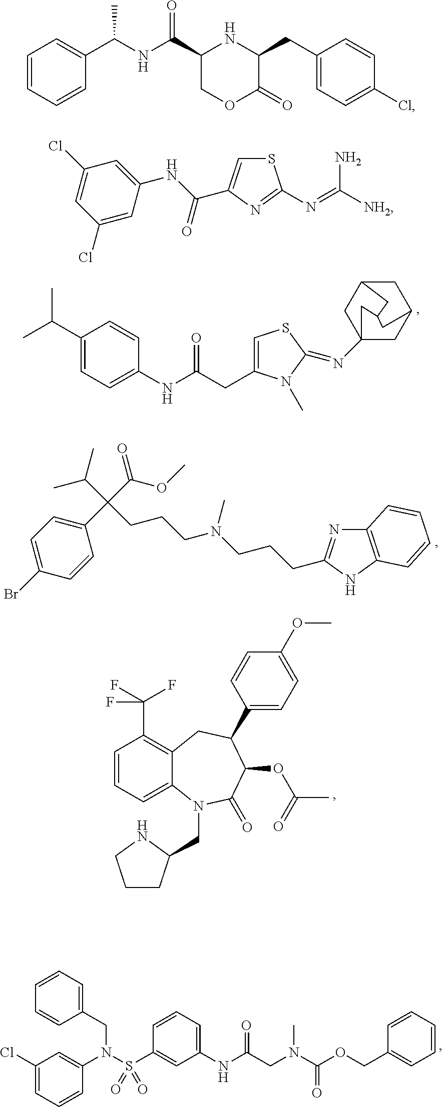 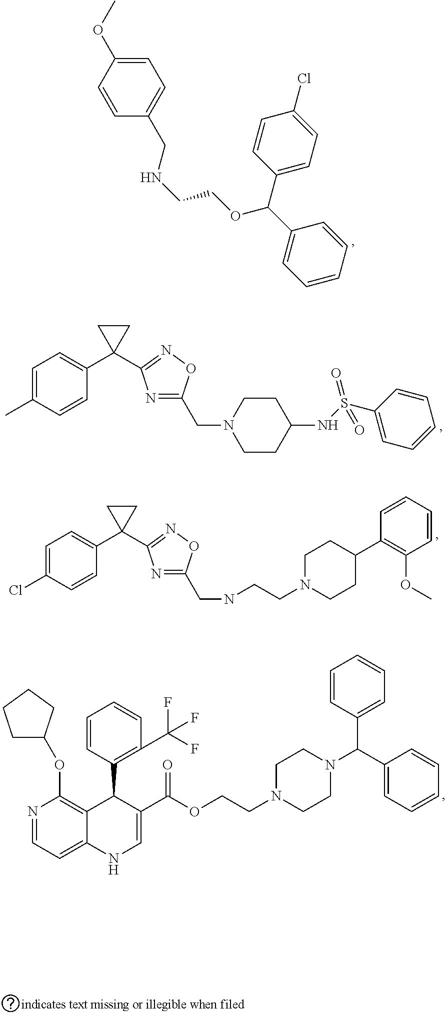
N-[(1R)-1-(4-fluorophenyl)-2-methylpropyl]-1-[2-(3-methoxyphenyl)ethyl]piperidine-4-carboxamide, 6-fluoro-1-isopropyl-2-{[1-(2-phenylethyl)piperidin-4-yl)carbonyl-1,2,3,4-tetrahydroisoquinoline, diltiazem, tylerdipine, P-11520031, DP-3005, RQ-00311610, A-1264087, A-1315647, VMD-3816 or VMD-3222, or a pharmaceutically acceptable salt thereof,
wherein the dosage of the compound is about 10 mg or more; or
the compound is administered concurrently with an anti-epileptic drug (e.g, ethosuximide, valproic acid, or lamotrigine); or
the genetic epilepsy is a refractory genetic epilepsy.

33. A method of modulating a T-type calcium channel in a subject, wherein the method comprises orally administering to the subject a compound selected from: 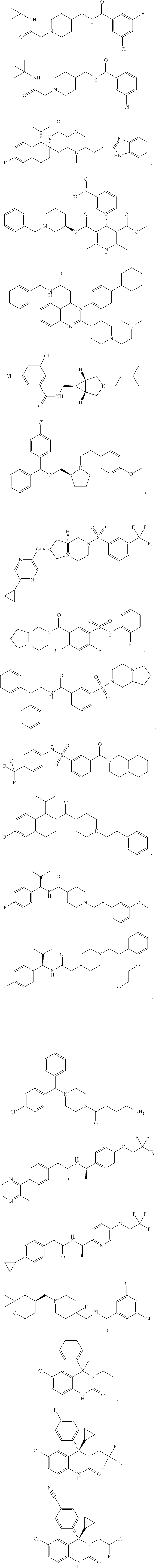 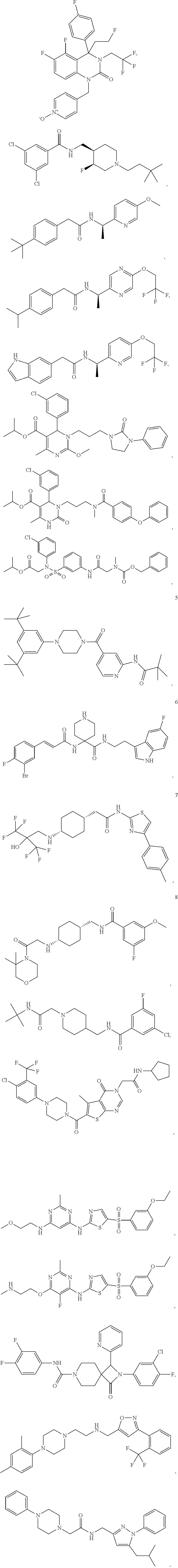 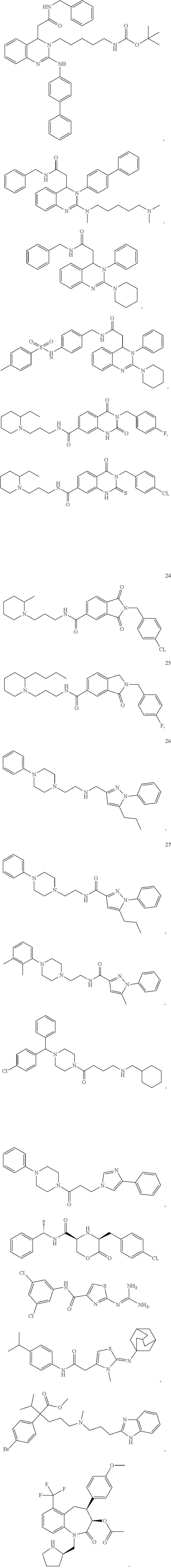 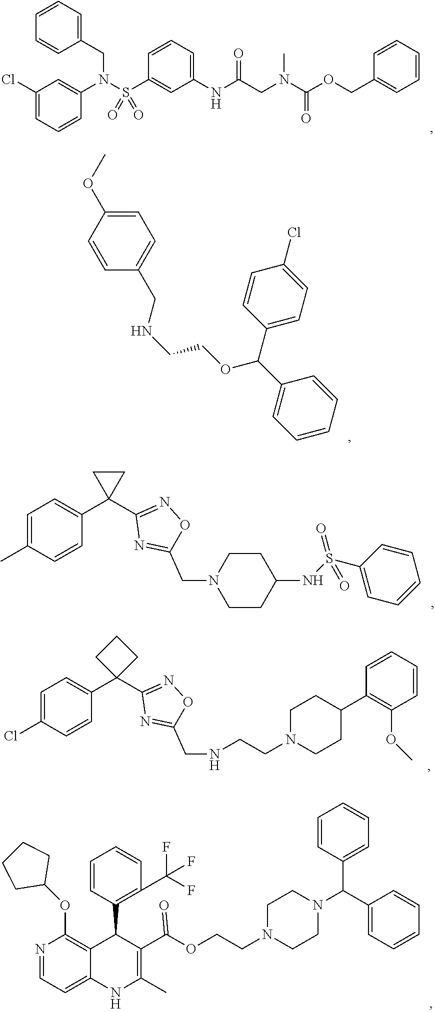
N-[(1R)-1-(4-fluorophenyl)-2-methylpropyl]-1-[2-(3-methoxyphenyl)ethyl]piperidine-4-carboxamide, 6-fluoro-1-isopropyl-2-{[1-(2-phenylethyl)piperidin-4-ylcarbonyl-1,2,3,4-tetrahydroisoquinoline, diltiazem, tylerdipine, P-11520031, DP-3005, RQ-00311610, A-1264087, A-1315647, VMD-3816 or VMD-3222, or a pharmaceutically acceptable salt thereof,
wherein the dosage of the compound is about 10 mg or more; or
the compound is administered concurrently with an anti-epileptic drug (e.g, ethosuximide, valproic acid, or lamotrigine).

34. A method of enhancing the potency of an inactivated T-type calcium channel in a subject (e.g., relative to a reference standard), wherein the method comprises orally administering to the subject a compound selected from: 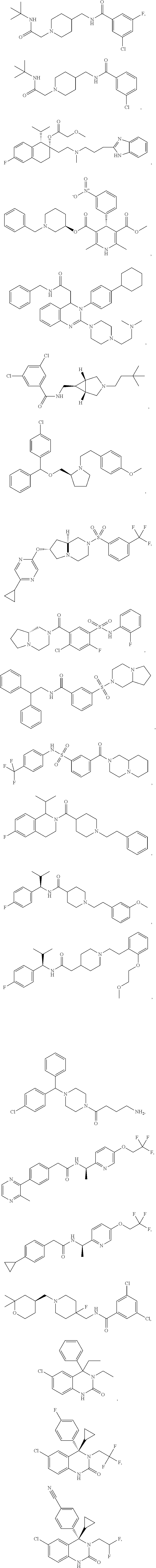 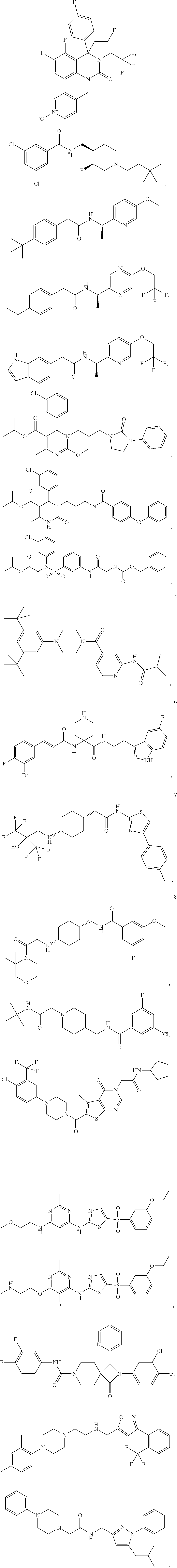 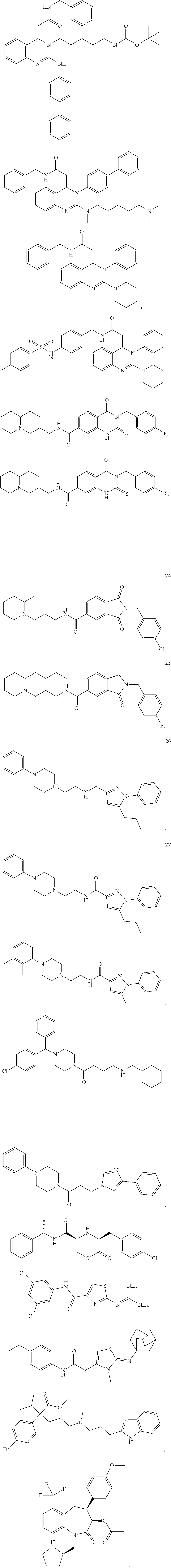 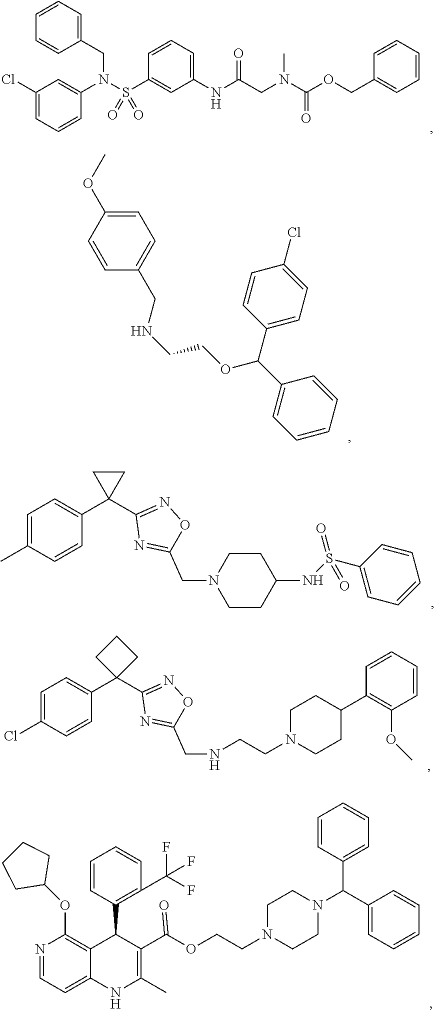
N-[(1R)-1-(4-fluorophenyl)-2-methylpropyl]-1-[2-(3-methoxyphenyl)ethyl]piperidine-4-carboxamide, 6-fluoro-1-isopropyl-2-{[1-(2-phenylethyl)piperidin-4-yl)carbonyl-1,2,3,4-tetrahydroisoquinoline, diltiazem, tylerdipine, P-11520031, DP-3005, RQ-00311610, A-1264087, A-1315647, VMD-3816 or VMD-3222, or a pharmaceutically acceptable salt thereof,
wherein the dosage of the compound is about 10 mg or more; or
the compound is administered concurrently with an anti-epileptic drug (e.g, ethosuximide, valproic acid, or lamotrigine).

35. A method of treating status epilepticus in a subject in need thereof, wherein the method comprises intravenously or intramuscularly administering to the subject a compound selected from: 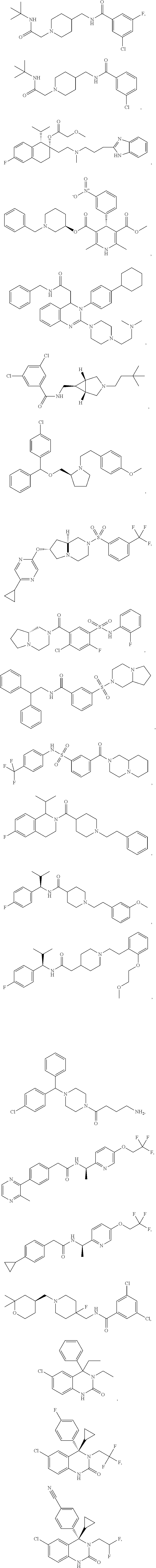 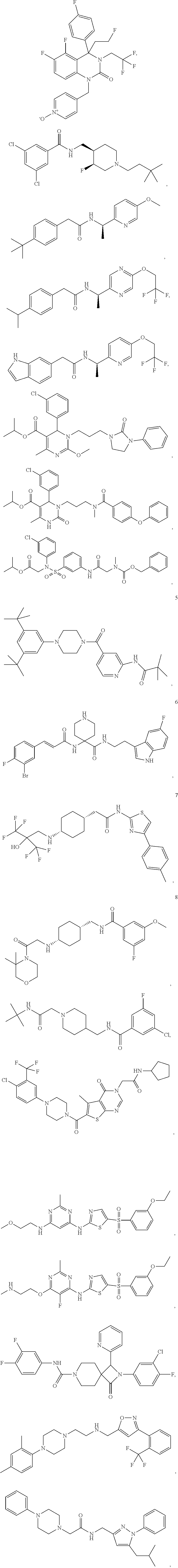 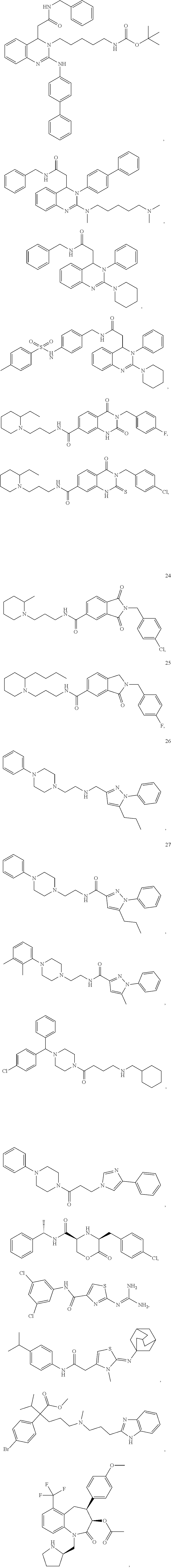 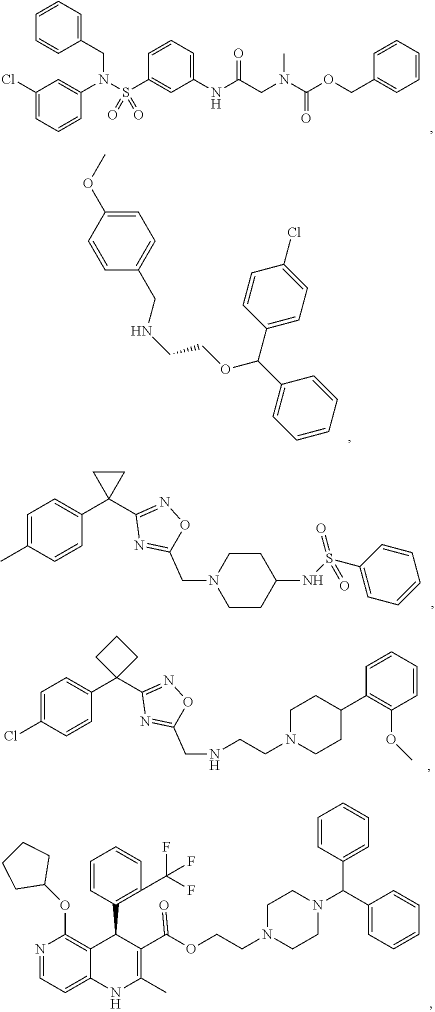
N-[(1R)-1-(4-fluorophenyl)-2-methylpropyl]-1-[2-(3-methoxyphenyl)ethyl]piperidine-4-carboxamide, 6-fluoro-1-isopropyl-2-{[1-(2-phenylethyl)piperidin-4-yl)carbonyl-1,2,3,4-tetrahydroisoquinoline, diltiazem, tylerdipine, P-11520031, DP-3005, RQ-00311610, A-1264087, A-1315647, VMD-3816 or VMD-3222, or a pharmaceutically acceptable salt thereof, wherein the dosage of the compound is about 10 mg or more; or
the compound is administered concurrently with an anti-epileptic drug (e.g, ethosuximide, valproic acid, or lamotrigine).

36. A method of treating a mood disorder in a subject in need thereof, wherein the method comprises orally administering to the subject a compound selected from: 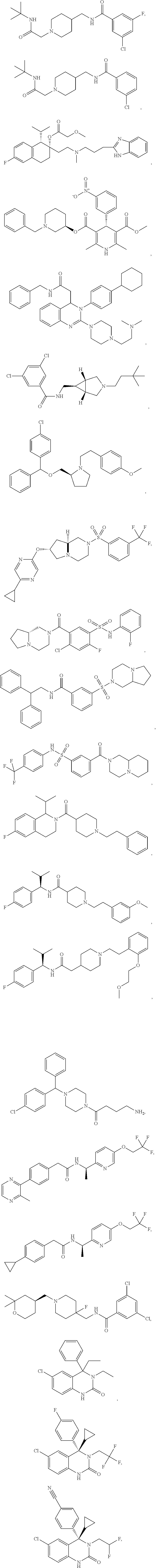 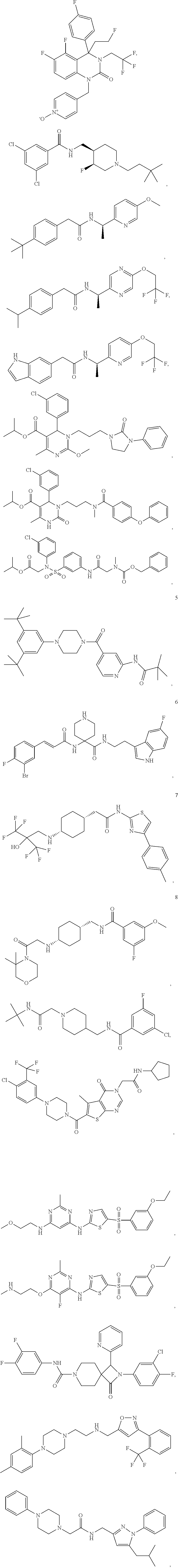 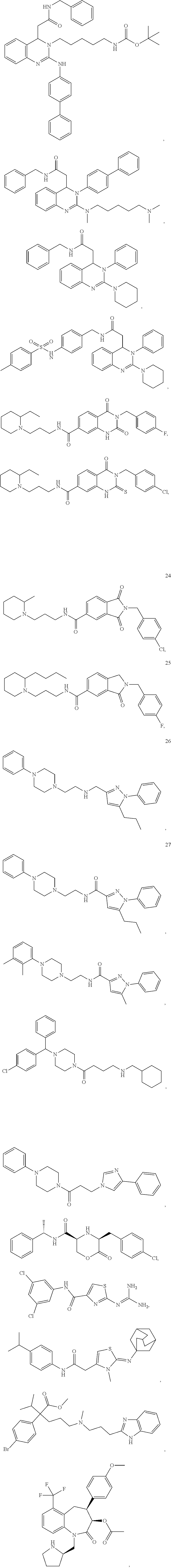 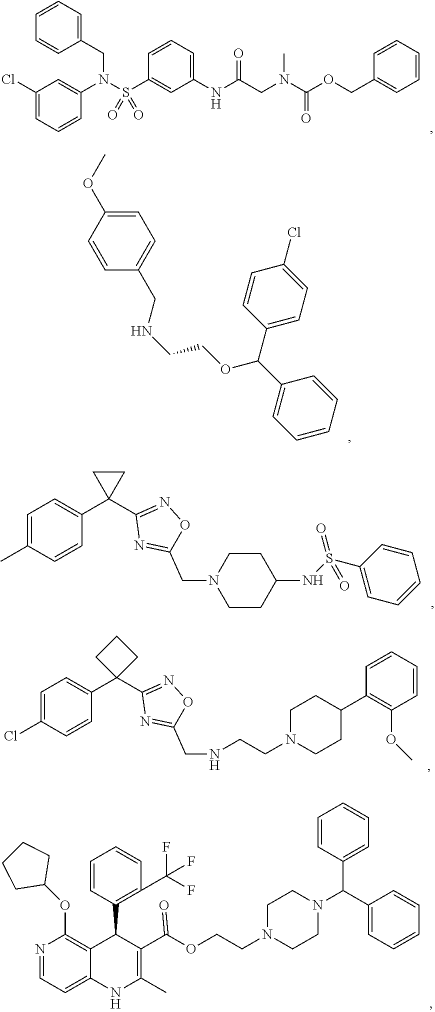
N-[(1R)-1-(4-fluorophenyl)-2-methylpropyl]-1-[2-(3-methoxyphenyl)ethyl]piperidine-4-carboxamide, 6-fluoro-1-isopropyl-2-{[1-(2-phenylethyl)piperidin-4-yl)carbonyl-1,2,3,4-tetrahydroisoquinoline, diltiazem, tylerdipine, P-11520031, DP-3005, RQ-00311610, A-1264087, A-1315647, VMD-3816 or VMD-3222, or a pharmaceutically acceptable salt thereof, wherein the dosage of the compound is about 10 mg or more; or
the compound is administered concurrently with an anti-epileptic drug (e.g, ethosuximide, valproic acid, or lamotrigine).

37. The method of claim 36, wherein the mood disorder is selected from depression, major depressive disorder, bipolar disorder, dysthymic disorder, anxiety disorders, stress, post-traumatic stress disorder, bipolar disorder, and compulsive disorders.

38. The method of any one of claims 31-37, wherein the compound is 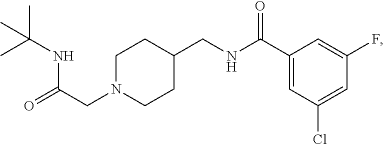 [SMILES: CC(C)(C)NC(=O)CN1CCC(CNC(=O)c2cc(F)cc(Cl)c2)CC1]
or a pharmaceutically acceptable salt thereof.

39. The method of any one of claims 31-38, wherein the subject experiences at least one seizure per day.

40. The method of claim 39, wherein the subject experiences at least one seizure per day comprising at least 1-6 Hz (e.g., 3-4 Hz) SWD.

41. The method of claim 40, wherein the subject experiences at least one seizure per day lasting about 5 seconds or more (e.g., about 10 seconds or more, about 15 seconds or more, about 20 seconds or more, about 30 seconds or more, or about 1 minute or more).

42. The method of any one of claims 31-41, wherein the subject experiences a decrease in seizure SWDs upon administration of the compound.

43. The method of any one of claims 31-41, wherein the compound is administered concurrently with an anti-epileptic drug (e.g, ethosuximide, valproic acid, or lamotrigine).

44. The method of any one of claims 31-43, wherein the concurrent administration comprises simultaneous administration, or administration of the compound before or after an anti-epileptic drug (e.g., ethosuximide, valproic acid, or lamotrigine).

45. The method of any one of claims 31-32 and 38-44, wherein the juvenile myoclonic epilepsy or genetic epilepsy are refractory.

46. The method of any one of claims 31-43, wherein the absence seizures are refractory to an anti-epileptic drug (e.g., ethosuximide, valproic acid, or lamotrigine).

47. The method of any one of claims 31-46, wherein the compound is administered daily.

48. The method of claim 47, wherein the compound is administered twice daily.

49. The method of claim 47, wherein the compound is administered daily for at least one week.

50. The method of claim 47, wherein the compound is administered daily for more than one week.

51. The method of any one of claims 31-50, wherein the dosage of the compound is greater than 10 mg.

52. The method of any one of claims 31-51, wherein the dosage of the compound is about 20 mg.

53. The method The method of any one of claims 31-51, wherein the dosage of the compound is about 40 mg.

54. The method of any one of claims 31-51, wherein the dosage of the compound is about 60 mg.

55. The method of any one of claims 33-54, wherein the subject has epilepsy.

56. The method of any one of claims 31-55, wherein the subject is a mammal (e.g., a human).

57. The method of any one of claims 31-56, wherein the subject is an adult (e.g., male or female).

58. The method of any one of claims 31-56, wherein the subject is a child.

59. The method of any one of claims 31-58, further comprising analyzing or receiving analysis of an EEG recording at least once prior to the end of treatment.

60. The method of claim 59, wherein the dosage is adjusted (e.g., increased) based on analysis of an EEG recording.

61. The method of any one of claims 31-60, further comprising analyzing or receiving analysis of a blood sample from the subject at least once prior to the end of treatment.

62. The method of claim 61, wherein the dosage is adjusted (e.g., increased) based on analysis of a blood sample.

63. The method of any one of claims 31-62, wherein the compound is administered daily for one week at a dosage of 20 mg, then administered daily for a week at a dosage greater than 20 mg (e.g., 25 mg, 30 mg, 35 mg, 40 mg, 45 mg, or 50 mg).

64. The method of any one of claims 31-63, wherein the compound is administered daily for one week at a dosage of 20 mg, then administered daily for a week at a dosage of 40 mg, then administered daily for a week at a dosage greater than 40 mg (e.g., 45 mg, 50 mg, 55 mg, 60 mg, 65 mg, or 70 mg).

65. The method of any one of the preceding claims, wherein the compound has selectivity for T-type calcium channel Cav3.2 compared with T-type calcium channel Cav3.1 or T-type calcium channel Cav3.3.

66. The method of any one of claims 1-63, wherein the compound has selectivity for T-type calcium channel Cav3.1 compared with T-type calcium channel Cav3.2 or T-type calcium channel Cav3.3.

67. The method of any one of claims 1-63, wherein the compound has selectivity for T-type calcium channel Cav3.3 compared with T-type calcium channel Cav3.1 or T-type calcium channel Cav3.2.
